SEQUENCE LISTING (25 sequences)

SEQ ID NO: 1 - protein, 921 aa, Artificial Sequence
  Synthetic
MGSSHHHHHHSSGLVPRGSHMMVKQGVFMKTDQSKVKKLSDYKSLDYFVIHVDLQIDLSKKPVESKARLTVVPNLNVDSHSNDLVLDGENMTLVSLQMNDNLLKENEYELTKDSLIIKNIPQNTPFTIEMTSLLGENTDLFGLYETEGVALVKAESEGLRRVFYLPDRPDNLATYKTTIIANQEDYPVLLSNGVLIEKKELPLGLHSVTWLDDVPKPSYLFALVAGNLQRSVTYYQTKSGRELPIEFYVPPSATSKCDFAKEVLKEAMAWDERTFNLECALRQHMVAGVDKYASGASEPTGLNLFNTENLFASPETKTDLGILRVLEVVAHEFFHYWSGDRVTIRDWFNLPLKEGLTTFRAAMFREELFGTDLIRLLDGKNLDERAPRQSAYTAVRSLYTAAAYEKSADIFRMMMLFIGKEPFIEAVAKFFKDNDGGAVTLEDFIESISNSSGKDLRSFLSWFTESGIPELIVTDELNPDTKQYFLKIKTVNGRNRPIPILMGLLDSSGAEIVADKLLIVDQEEIEFQFENIQTRPIPSLLRSFSAPVHMKYEYSYQDLLLLMQFDTNLYNRCEAAKQLISALINDFCIGKKIELSPQFFAVYKALLSDNSLNEWMLAELITLPSLEELIENQDKPDFEKLNEGRQLIQNALANELKTDFYNLLFRIQISGDDDKQKLKGFDLKQAGLRRLKSVCFSYLLNVDFEKTKEKLILQFEDALGKNMTETALALSMLCEINCEEADVALEDYYHYWKNDPGAVNNWFSIQALAHSPDVIERVKKLMRHGDFDLSNPNKVYALLGSFIKNPFGFHSVTGEGYQLVADAIFDLDKINPTLAANLTEKFTYWDKYDVNRQAMMISTLKIIYSNATSSDVRTMAKKGLDKVKEDLPLPIHLTFHGGSTMQDRTAQLIADGNKENAYQLH

SEQ ID NO: 2 - protein, 273 aa, Artificial Sequence
  Synthetic
MAHHHHHHMGTAISIKTPEDIEKMRVAGRLAAEVLEMIEPYVKPGVSTGELDRICNDYIVNEQHAVSACLGYHGYPKSVCISINEVVCHGIPDDAKLLKDGDIVNIDVTVIKDGFHGDTSKMFIVGKPTIMGERLCRITQESLYLALRMVKPGINLREIGAAIQKFVEAEGFSVVREYCGHGIGRGFHEEPQVLHYDSRETNVVLKPGMTFTIEPMVNAGKKEIRTMKDGWTVKTKDRSLSAQYEHTIVVTDNGCEILTLRKDDTIPAIISHD

SEQ ID NO: 3 - protein, 330 aa, Artificial Sequence
  Synthetic
MAHHHHHHMGTLEANTNGPGSMLSRMPVSSRTVPFGDHETWVQVTTPENAQPHALPLIVLHGGPGMAHNYVANIAALADETGRTVIHYDQVGCGNSTHLPDAPADFWTPQLFVDEFHAVCTALGIERYHVLGQSWGGMLGAEIAVRQPSGLVSLAICNSPASMRLWSEAAGDLRAQLPAETRAALDRHEAAGTITHPDYLQAAAEFYRRHVCRVVPTPQDFADSVAQMEAEPTVYHTMNGPNEFHVVGTLGDWSVIDRLPDVTAPVLVIAGEHDEATPKTWQPFVDHIPDVRSHVFPGTSHCTHLEKPEEFRAVVAQFLHQHDLAADARV

SEQ ID NO: 4 - protein, 452 aa, Artificial Sequence
  Synthetic
MTQQEYQNRRQALLAKMAPGSAAIIFAAPEATRSADSEYPYRQNSDFSYLTGFNEPEAVLILVKSDETHNHSVLFNRIRDLTAEIWFGRRLGQEAAPTKLAVDRALPFDEINEQLYLLLNRLDVIYHAQGQYAYADNIVFAALEKLRHGFRKNLRAPATLTDWRPWLHEMRLFKSAEEIAVLRRAGEISALAHTRAMEKCRPGMFEYQLEGEILHEFTRHGARYPAYNTIVGGGENGCILHYTENECELRDGDLVLIDAGCEYRGYAGDITRTFPVNGKFTPAQRAVYDIVLAAINKSLTLFRPGTSIREVTEEVVRIMVVGLVELGILKGDIEQLIAEQAHRPFFMHGLSHWLGMDVHDVGDYGSSDRGRILEPGMVLTVEPGLYIAPDADVPPQYRGIGIRIEDDIVITATGNENLTASVVKDPDDIEALMALNHAGENLYFQEHHHHHH

SEQ ID NO: 5 - protein, 303 aa, Artificial Sequence
  Synthetic
MDTEKLMKAGEIAKKVREKAIKLARPGMLLLELAESIEKMIMELGGKPAFPVNLSINEIAAHYTPYKGDTTVLKEGDYLKIDVGVHIDGFIADTAVTVRVGMEEDELMEAAKEALNAAISVARAGVEIKELGKAIENEIRKRGFKPIVNLSGHKIERYKLHAGISIPNIYRPHDNYVLKEGDVFAIEPFATIGAGQVIEVPPTLIYMYVRDVPVRVAQARFLLAKIKREYGTLPFAYRWLQNDMPEGQLKLALKTLEKAGAIYGYPVLKEIRNGIVAQFEHTIIVEKDSVIVTQDMINKSTLE

SEQ ID NO: 6 - protein, 428 aa, Artificial Sequence
  Synthetic
HMSSPLHYVLDGIHCEPHFFTVPLDHQQPDDEETITLFGRTLCRKDRLDDELPWLLYLQGGPGFGAPRPSANGGWIKRALQEFRVLLLDQRGTGHSTPIHAELLAHLNPRQQADYLSHFRADSIVRDAELIREQLSPDHPWSLLGQSFGGFCSLTYLSLFPDSLHEVYLTGGVAPIGRSADEVYRATYQRVADKNRAFFARFPHAQAIANRLATHLQRHDVRLPNGQRLTVEQLQQQGLDLGASGAFEELYYLLEDAFIGEKLNPAFLYQVQAMQPFNTNPVFAILHELIYCEGAASHWAAERVRGEFPALAWAQGKDFAFTGEMIFPWMFEQFRELIPLKEAAHLLAEKADWGPLYDPVQLARNKVPVACAVYAEDMYVEFDYSRETLKGLSNSRAWITNEYEHNGLRVDGEQILDRLIRLNRDCLE

SEQ ID NO: 7 - protein, 348 aa, Artificial Sequence
  Synthetic
MKERLEKLVKFMDENSIDRVFIAKPVNVYYFSGTSPLGGGYIIVDGDEATLYVPELEYEMAKEESKLPVVKFKKFDEIYEILKNTETLGIEGTLSYSMVENFKEKSNVKEFKKIDDVIKDLRIIKTKEEIEIIEKACEIADKAVMAAIEEITEGKREREVAAKVEYLMKMNGAEKPAFDTIIASGHRSALPHGVASDKRIERGDLVVIDLGALYNHYNSDITRTIVVGSPNEKQREIYEIVLEAQKRAVEAAKPGMTAKELDSIAREIIKEYGYGDYFIHSLGHGVGLEIHEWPRISQYDETVLKEGMVITIEPGIYIPKLGGVRIEDTVLITENGAKRLTKTERELL

SEQ ID NO: 8 - protein, 298 aa, Artificial Sequence
  Synthetic
MIPITTPVGNFKVWTKRFGTNPKIKVLLLHGGPAMTHEYMECFETFFQREGFEFYEYDQLGSYYSDQPTDEKLWNIDRFVDEVEQVRKAIHADKENFYVLGNSWGGILAMEYALKYQQNLKGLIVANMMASAPEYVKYAEVLSKQMKPEVLAEVRAIEAKKDYANPRYTELLFPNYYAQHICRLKEWPDALNRSLKHVNSTVYTLMQGPSELGMSSDARLAKWDIKNRLHEIATPTLMIGARYDTMDPKAMEEQSKLVQKGRYLYCPNGSHLAMWDDQKVFMDGVIKFIKDVDTKSFN

SEQ ID NO: 9 - protein, 428 aa, Artificial Sequence; identical to SEQ ID NO: 6
  Synthetic

SEQ ID NO: 10 - protein, 310 aa, Artificial Sequence
  Synthetic
MYEIKQPFHSGYLQVSEIHQIYWEESGNPDGVPVIFLHGGPGAGASPECRGFFNPDVFRIVIIDQRGCGRSHPYACAEDNTTWDLVADIEKVREMLGIGKWLVFGGSWGSTLSLAYAQTHPERVKGLVLRGIFLCRPSETAWLNEAGGVSRIYPEQWQKFVAPIAENRRNRLIEAYHGLLFHQDEEVCLSAAKAWADWESYLIRFEPEGVDEDAYASLAIARLENHYFVNGGWLQGDKAILNNIGKIRHIPTVIVQGRYDLCTPMQSAWELSKAFPEAELRVVQAGHCAFDPPLADALVQAVEDILPRLL

SEQ ID NO: 11 - protein, 891 aa, Artificial Sequence
  Synthetic
MGSSHHHHHHSSGENLYFQGHMTQQPQAKYRHDYRAPDYQITDIDLTFDLDAQKTVVTAVSQAVRHGASDAPLRLNGEDLKLVSVHINDEPWTAWKEEEGALVISNLPERFTLKIINEISPAANTALEGLYQSGDALCTQCEAEGFRHITYYLDRPDVLARFTTKIIADKIKYPFLLSNGNRVAQGELENGRHWVQWQDPFPKPCYLFALVAGDFDVLRDTFTTRSGREVALELYVDRGNLDRAPWAMTSLKNSMKWDEERFGLEYDLDIYMIVAVDFFNMGAMENKGLNIFNSKYVLARTDTATDKDYLDIERVIGHEYFHNWTGNRVTCRDWFQLSLKEGLTVFRDQEFSSDLGSRAVNRINNVRTMRGLQFAEDASPMAHPIRPDMVIEMNNFYTLTVYEKGAEVIRMIHTLLGEENFQKGMQLYFERHDGSAATCDDFVQAMEDASNVDLSHFRRWYSQSGTPIVTVKDDYNPETEQYTLTISQRTPATPDQAEKQPLHIPFAIELYDNEGKVIPLQKGGHPVNSVLNVTQAEQTFVFDNVYFQPVPALLCEFSAPVKLEYKWSDQQLTFLMRHARNDFSRWDAAQSLLATYIKLNVARHQQGQPLSLPVHVADAFRAVLLDEKIDPALAAEILTLPSVNEMAELFDIIDPIAIAEVREALTRTLATELADELLAIYNANYQSEYRVEHEDIAKRTLRNACLRFLAFGETHLADVLVSKQFHEANNMTDALAALSAAVAAQLPCRDALMQEYDDKWHQNGLVMDKWFILQATSPAANVLETVRGLLQHRSFTMSNPNRIRSLIGAFAGSNPAAFHAEDGSGYLFLVEMLTDLNSRNPQVASRLIEPLIRLKRYDAKRQEKMRAALEQLKGLENLSGDLYEKITKALA

SEQ ID NO: 12 - protein, 889 aa, Artificial Sequence
  Synthetic
PKIHYRKDYKPSGFIINQVTLNINIHDQETIVRSVLDMDISKHNVGEDLVFDGVGLKINEISINNKKLVEGEEYTYDNEFLTIFSKFVPKSKFAFSSEVIIHPETNYALTGLYKSKNIIVSQCEATGFRRITFFIDRPDMMAKYDVTVTADKEKYPVLLSNGDKVNEFEIPGGRHGARFNDPPLKPCYLFAVVAGDLKHLSATYITKYTKKKVELYVFSEEKYVSKLQWALECLKKSMAFDEDYFGLEYDLSRLNLVAVSDFNVGAMENKGLNIFNANSLLASKKNSIDFSYARILTVVGHEYFHQYTGNRVTLRDWFQLTLKEGLTVHRENLFSEEMTKTVTTRLSHVDLLRSVQFLEDSSPLSHPIRPESYVSMENFYTTTVYDKGSEVMRMYLTILGEEYYKKGFDIYIKKNDGNTATCEDFNYAMEQAYKMKKADNSANLNQYLLWFSQSGTPHVSFKYNYDAEKKQYSIHVNQYTKPDENQKEKKPLFIPISVGLINPENGKEMISQTTLELTKESDTFVFNNIAVKPIPSLFRGFSAPVYIEDQLTDEERILLLKYDSDAFVRYNSCTNIYMKQILMNYNEFLKAKNEKLESFQLTPVNAQFIDAIKYLLEDPHADAGFKSYIVSLPQDRYIINFVSNLDTDVLADTKEYIYKQIGDKLNDVYYKMFKSLEAKADDLTYFNDESHVDFDQMNMRTLRNTLLSLLSKAQYPNILNEIIEHSKSPYPSNWLTSLSVSAYFDKYFELYDKTYKLSKDDELLLQEWLKTVSRSDRKDIYEILKKLENEVLKDSKNPNDIRAVYLPFTNNLRRFHDISGKGYKLIAEVITKTDKFNPMVATQLCEPFKLWNKLDTKRQELMLNEMNTMLQEPQISNNLKEYLLRLTNK

SEQ ID NO: 13 - protein, 932 aa, Artificial Sequence
  Synthetic
MGSSHHHHHHSSGMWLAAAAPSLARRLLFLGPPPPPLLLLVFSRSSRRRLHSLGLAAMPEKRPFERLPADVSPINYSLCLKPDLLDFTFEGKLEAAAQVRQATNQIVMNCADIDIITASYAPEGDEEIHATGFNYQNEDEKVTLSFPSTLQTGTGTLKIDFVGELNDKMKGFYRSKYTTPSGEVRYAAVTQFEATDARRAFPCWDEPAIKATFDISLVVPKDRVALSNMNVIDRKPYPDDENLVEVKFARTPVMSTYLVAFVVGEYDFVETRSKDGVCVRVYTPVGKAEQGKFALEVAAKTLPFYKDYFNVPYPLPKIDLIAIADFAAGAMENWGLVTYRETALLIDPKNSCSSSRQWVALVVGHELAHQWFGNLVTMEWWTHLWLNEGFASWIEYLCVDHCFPEYDIWTQFVSADYTRAQELDALDNSHPIEVSVGHPSEVDEIFDAISYSKGASVIRMLHDYIGDKDFKKGMNMYLTKFQQKNAATEDLWESLENASGKPIAAVMNTWTKQMGFPLIYVEAEQVEDDRLLRLSQKKFCAGGSYVGEDCPQWMVPITISTSEDPNQAKLKILMDKPEMNVVLKNVKPDQWVKLNLGTVGFYRTQYSSAMLESLLPGIRDLSLPPVDRLGLQNDLFSLARAGIISTVEVLKVMEAFVNEPNYTVWSDLSCNLGILSTLLSHTDFYEEIQEFVKDVFSPIGERLGWDPKPGEGHLDALLRGLVLGKLGKAGHKATLEEARRRFKDHVEGKQILSADLRSPVYLTVLKHGDGTTLDIMLKLHKQADMQEEKNRIERVLGATLLPDLIQKVLTFALSEEVRPQDTVSVIGGVAGGSKHGRKAAWKFIKDNWEELYNRYQGGFLISRLIKLSVEGFAVDKMAGEVKAFFESHPAPSAERTIQQCCENILLNAAWLKRDAESIHQYLLQRKASPPTV

SEQ ID NO: 14 - protein, 932 aa, Artificial Sequence
  Synthetic
MGSSHHHHHHSSGMWLAAAAPSLARRLLFLGPPPPPLLLLVFSRSSRRRLHSLGLAAMPEKRPFERLPADVSPINYSLCLKPDLLDFTFEGKLEAAAQVRQATNQIVMNCADIDIITASYAPEGDEEIHATGFNYQNEDEKVTLSFPSTLQTGTGTLKIDFVGELNDKMKGFYRSKYTTPSGEVRYAAVTQFEATDARRAFPCWDEPAIKATFDISLVVPKDRVALSNMNVIDRKPYPDDENLVEVKFARTPVMSTYLVAFVVGEYDFVETRSKDGVCVRVYTPVGKAEQGKFALEVAAKTLPFYKDYFNVPYPLPKIDLIAIADFAAGAMENWGLVTYRETALLIDPKNSCSSSRQWVALVVGHVLAHQWFGNLVTMEWWTHLWLNEGFASWIEYLCVDHCFPEYDIWTQFVSADYTRAQELDALDNSHPIEVSVGHPSEVDEIFDAISYSKGASVIRMLHDYIGDKDFKKGMNMYLTKFQQKNAATEDLWESLENASGKPIAAVMNTWTKQMGFPLIYVEAEQVEDDRLLRLSQKKFCAGGSYVGEDCPQWMVPITISTSEDPNQAKLKILMDKPEMNVVLKNVKPDQWVKLNLGTVGFYRTQYSSAMLESLLPGIRDLSLPPVDRLGLQNDLFSLARAGIISTVEVLKVMEAFVNEPNYTVWSDLSCNLGILSTLLSHTDFYEEIQEFVKDVFSPIGERLGWDPKPGEGHLDALLRGLVLGKLGKAGHKATLEEARRRFKDHVEGKQILSADLRSPVYLTVLKHGDGTTLDIMLKLHKQADMQEEKNRIERVLGATLLPDLIQKVLTFALSEEVRPQDTVSVIGGVAGGSKHGRKAAWKFIKDNWEELYNRYQGGFLISRLIKLSVEGFAVDKMAGEVKAFFESHPAPSAERTIQQCCENILLNAAWLKRDAESIHQYLLQRKASPPTV

SEQ ID NO: 15 - protein, 864 aa, Artificial Sequence
  Synthetic
MIYEFVMTDPKIKYLKDYKPSNYLIDETHLIFELDESKTRVTANLYIVANRENRENNTLVLDGVELKLLSIKLNNKHLSPAEFAVNENQLIINNVPEKFVLQTVVEINPSANTSLEGLYKSGDVFSTQCEATGFRKITYYLDRPDVMAAFTVKIIADKKKYPIILSNGDKIDSGDISDNQHFAVWKDPFKKPCYLFALVAGDLASIKDTYITKSQRKVSLEIYAFKQDIDKCHYAMQAVKDSMKWDEDRFGLEYDLDTFMIVAVPDFNAGAMENKGLNIFNTKYIMASNKTATDKDFELVQSVVGHEYFHNWTGDRVTCRDWFQLSLKEGLTVFRDQEFTSDLNSRDVKRIDDVRIIRSAQFAEDASPMSHPIRPESYIEMNNFYTVTVYNKGAEIIRMIHTLLGEEGFQKGMKLYFERHDGQAVTCDDFVNAMADANNRDFSLFKRWYAQSGTPNIKVSENYDASSQTYSLTLEQTTLPTADQKEKQALHIPVKMGLINPEGKNIAEQVIELKEQKQTYTFENIAAKPVASLFRDFSAPVKVEHKRSEKDLLHIVKYDNNAFNRWDSLQQIATNIILNNADLNDEFLNAFKSILHDKDLDKALISNALLIPIESTIAEAMRVIMVDDIVLSRKNVVNQLADKLKDDWLAVYQQCNDNKPYSLSAEQIAKRKLKGVCLSYLMNASDQKVGTDLAQQLFDNADNMTDQQTAFTELLKSNDKQVRDNAINEFYNRWRHEDLVVNKWLLSQAQISHESALDIVKGLVNHPAYNPKNPNKVYSLIGGFGANFLQYHCKDGLGYAFMADTVLALDKFNHQVAARMARNLMSWKRYDSDRQAMMKNALEKIKASNPSKNVFEIVSKSLES

SEQ ID NO: 16 - protein, 366 aa, Artificial Sequence
  Synthetic
MGSSHHHHHHSSGMEVRNMVDYELLKKVVEAPGVSGYEFLGIRDVVIEEIKDYVDEVKVDKLGNVIAHKKGEGPKVMIAAHMDQIGLMVTHIEKNGFLRVAPIGGVDPKTLIAQRFKVWIDKGKFIYGVGASVPPHIQKPEDRKKAPDWDQIFIDIGAESKEEAEDMGVKIGTVITWDGRLERLGKHRFVSIAFDDRIAVYTILEVAKQLKDAKADVYFVATVQEEVGLRGARTSAFGIEPDYGFAIDVTIAADIPGTPEHKQVTHLGKGTAIKIMDRSVICHPTIVRWLEELAKKHEIPYQLEILLGGGTDAGAIHLTKAGVPTGALSVPARYIHSNTEVVDERDVDATVELMTKALENIHELKI

SEQ ID NO: 17 - protein, 408 aa, Artificial Sequence
  Synthetic
MDAFTENLNKLAELAIRVGLNLEEGQEIVATAPIEAVDFVRLLAEKAYENGASLFTVLYGDNLIARKRLALVPEAHLDRAPAWLYEGMAKAFHEGAARLAVSGNDPKALEGLPPERVGRAQQAQSRAYRPTLSAITEFVTNWTIVPFAHPGWAKAVFPGLPEEEAVQRLWQAIFQATRVDQEDPVAAWEAHNRVLHAKVAFLNEKRFHALHFQGPGTDLTVGLAEGHLWQGGATPTKKGRLCNPNLPTEEVFTAPHRERVEGVVRASRPLALSGQLVEGLWARFEGGVAVEVGAEKGEEVLKKLLDTDEGARRLGEVALVPADNPIAKTGLVFFDTLFDENAASHIAFGQAYAENLEGRPSGEEFRRRGGNESMVHVDWMIGSEEVDVDGLLEDGTRVPLMRRGRWVI

SEQ ID NO: 18 - protein, 362 aa, Artificial Sequence
  Synthetic
MAKLDETLTMLKALTDAKGVPGNEREARDVMKTYIAPYADEVTTDGLGSLIAKKEGKSGGPKVMIAGHLDEVGFMVTQIDDKGFIRFQTLGGWWSQVMLAQRVTIVTKKGDITGVIGSKPPHILPSEARKKPVEIKDMFIDIGATSREEAMEWGVRPGDMIVPYFEFTVLNNEKMLLAKAWDNRIGCAVAIDVLKQLKGVDHPNTVYGVGTVQEEVGLRGARTAAQFIQPDIAFAVDVGIAGDTPGVSEKEAMGKLGAGPHIVLYDATMVSHRGLREFVIEVAEELNIPHHFDAMPGVGTDAGAIHLTGIGVPSLTIAIPTRYIHSHAAILHRDDYENTVKLLVEVIKRLDADKVKQLTFDE

SEQ ID NO: 19 - protein, 490 aa, Artificial Sequence
  Synthetic
MEDKVWISMGADAVGSLNPALSESLLPHSFASGSQVWIGEVAIDELAELSHTMHEQHNRCGGYMVHTSAQGAMAALMMPESIANFTIPAPSQQDLVNAWLPQVSADQITNTIRALSSFNNRFYTTTSGAQASDWLANEWRSLISSLPGSRIEQIKHSGYNQKSVVLTIQGSEKPDEWVIVGGHLDSTLGSHTNEQSIAPGADDDASGIASLSEIIRVLRDNNFRPKRSVALMAYAAEEVGLRGSQDLANQYKAQGKKVVSVLQLDMTNYRGSAEDIVFITDYTDSNLTQFLTTLIDEYLPELTYGYDRCGYACSDHASWHKAGFSAAMPFESKFKDYNPKIHTSQDTLANSDPTGNHAVKFTKLGLAYVIEMANAGSSQVPDDSVLQDGTAKINLSGARGTQKRFTFELSQSKPLTIQTYGGSGDVDLYVKYGSAPSKSNWDCRPYQNGNRETCSFNNAQPGIYHVMLDGYTNYNDVALKASTQHHHHHH

SEQ ID NO: 20 - protein, 494 aa, Artificial Sequence
  Synthetic
MEDKVWISIGSDASQTVKSVMQSNARSLLPESLASNGPVWVGQVDYSQLAELSHHMHEDHQRCGGYMVHSSPESAIAASNMPQSLVAFSIPEISQQDTVNAWLPQVNSQAITGTITSLTSFINRFYTTTSGAQASDWLANEWRSLSASLPNASVRQVSHFGYNQKSVVLTITGSEKPDEWIVLGGHLDSTIGSHTNEQSVAPGADDDASGIASVTEIIRVLSENNFQPKRSIAFMAYAAEEVGLRGSQDLANQYKAEGKQVISALQLDMTNYKGSVEDIVFITDYTDSNLTTFLSQLVDEYLPSLTYGFDTCGYACSDHASWHKAGFSAAMPFEAKFNDYNPMIHTPNDTLQNSDPTASHAVKFTKLGLAYAIEMASTTGGTPPPTGNVLKDGVPVNGLSGATGSQVHYSFELPAQKNLQISTAGGSGDVDLYVSFGSEATKQNWDCRPYRNGNNEVCTFAGATPGTYSIMLDGYRQFSGVTLKASTQHHHHHH

SEQ ID NO: 21 - protein, 877 aa, Artificial Sequence
  Synthetic
MTQQPQAKYRHDYRAPDYTITDIDLDFALDAQKTTVTAVSKVKRQGTDVTPLILNGEDLTLISVSVDGQAWPHYRQQDNTLVIEQLPADFTLTIVNDIHPATNSALEGLYLSGEALCTQCEAEGFRHITYYLDRPDVLARFTTRIVADKSRYPYLLSNGNRVGQGELDDGRHWVKWEDPFPKPSYLFALVAGDFDVLQDKFITRSGREVALEIFVDRGNLDRADWAMTSLKNSMKWDETRFGLEYDLDIYMIVAVDFFNMGAMENKGLNVFNSKYVLAKAETATDKDYLNIEAVIGHEYFHNWTGNRVTCRDWFQLSLKEGLTVFRDQEFSSDLGSRSVNRIENVRVMRAAQFAEDASPMAHAIRPDKVIEMNNFYTLTVYEKGSEVIRMMHTLLGEQQFQAGMRLYFERHDGSAATCDDFVQAMEDVSNVDLSLFRRWYSQSGTPLLTVHDDYDVEKQQYHLFVSQKTLPTADQPEKLPLHIPLDIELYDSKGNVIPLQHNGLPVHHVLNVTEAEQTFTFDNVAQKPIPSLLREFSAPVKLDYPYSDQQLTFLMQHARNEFSRWDAAQSLLATYIKLNVAKYQQQQPLSLPAHVADAFRAILLDEHLDPALAAQILTLPSENEMAELFTTIDPQAISTVHEAITRCLAQELSDELLAVYVANMTPVYRIEHGDIAKRALRNTCLNYLAFGDEEFANKLVSLQYHQADNMTDSLAALAAAVAAQLPCRDELLAAFDVRWNHDGLVMDKWFALQATSPAANVLVQVRTLLKHPAFSLSNPNRTRSLIGSFASGNPAAFHAADGSGYQFLVEILSDLNTRNPQVAARLIEPLIRLKRYDAGRQALMRKALEQLKTLDNLSGDLYEKITKALAAHHHHHH

SEQ ID NO: 22 - protein, 489 aa, Artificial Sequence
  Synthetic
MEEKVWISIGGDATQTALRSGAQSLLPENLINQTSVWVGQVPVSELATLSHEMHENHQRCGGYMVHPSAQSAMSVSAMPLNLNAFSAPEITQQTTVNAWLPSVSAQQITSTITTLTQFKNRFYTTSTGAQASNWIADHWRSLSASLPASKVEQITHSGYNQKSVMLTITGSEKPDEWVVIGGHLDSTLGSRTNESSIAPGADDDASGIAGVTEIIRLLSEQNFRPKRSIAFMAYAAEEVGLRGSQDLANRFKAEGKKVMSVMQLDMTNYQGSREDIVFITDYTDSNFTQYLTQLLDEYLPSLTYGFDTCGYACSDHASWHAVGYPAAMPFESKFNDYNPNIHSPQDTLQNSDPTGFHAVKFTKLGLAYVVEMGNASTPPTPSNQLKNGVPVNGLSASRNSKTWYQFELQEAGNLSIVLSGGSGDADLYVKYQTDADLQQYDCRPYRSGNNETCQFSNAQPGRYSILLHGYNNYSNASLVANAQHHHHHH

SEQ ID NO: 23 - protein, 488 aa, Artificial Sequence
  Synthetic
MEDKKVWISIGADAQQTALSSGAQPLLAQSVAHNGQAWIGEVSESELAALSHEMHENHHRCGGYIVHSSAQSAMAASNMPLSRASFIAPAISQQALVTPWISQIDSALIVNTIDRLTDFPNRFYTTTSGAQASDWIKQRWQSLSAGLAGASVTQISHSGYNQASVMLTIEGSESPDEWVVVGGHLDSTIGSRTNEQSIAPGADDDASGIAAVTEVIRVLAQNNFQPKRSIAFVAYAAEEVGLRGSQDVANQFKQAGKDVRGVLQLDMTNYQGSAEDIVFITDYTDNQLTQYLTQLLDEYLPTLNYGFDTCGYACSDHASWHQVGYPAAMPFEAKFNDYNPNIHTPQDTLANSDSEGAHAAKFTKLGLAYTVELANADSSPNPGNELKLGEPINGLSGARGNEKYFNYRLDQSGELVIRTYGGSGDVDLYVKANGDVSTGNWDCRPYRSGNDEVCRFDNATPGNYAVMLRGYRTYDNVSLIVEHHHHHH

SEQ ID NO: 24 - protein, 308 aa, Artificial Sequence
  Synthetic
GMPPITQQATVTAWLPQVDASQITGTISSLESFTNRFYTTTSGAQASDWIASEWQFLSASLPNASVKQVSHSGYNQKSVVMTITGSEAPDEWIVIGGHLDSTIGSHTNEQSVAPGADDDASGIAAVTEVIRVLSENNFQPKRSIAFMAYAAEEVGLRGSQDLANQYKSEGKNVVSALQLDMTNYKGSAQDVVFITDYTDSNFTQYLTQLMDEYLPSLTYGFDTCGYACSDHASWHNAGYPAAMPFESKFNDYNPRIHTTQDTLANSDPTGSHAKKFTQLGLAYAIEMGSATGDTPTPGNQLEHHHHHH

SEQ ID NO: 25 - protein, 354 aa, Artificial Sequence
  Synthetic
MVDWELMKKIIESPGVSGYEHLGIRDLVVDILKDVADEVKIDKLGNVIAHFKGSAPKVMVAAHMDKIGLMVNHIDKDGYLRVVPIGGVLPETLIAQKIRFFTEKGERYGVVGVLPPHLRREAKDQGGKIDWDSIIVDVGASSREEAEEMGFRIGTIGEFAPNFTRLSEHRFATPYLDDRICLYAMIEAARQLGEHEADIYIVASVQEEIGLRGARVASFAIDPEVGIAMDVTFAKQPNDKGKIVPELGKGPVMDVGPNINPKLRQFADEVAKKYEIPLQVEPSPRPTGTDANVMQINREGVATAVLSIPIRYMHSQVELADARDVDNTIKLAKALLEELKPMDFTPLEHHHHHH

(19) United States
(12) Patent Application Publication
(10) Pub. No.: US 2021/0148921 A1
(43) Pub. Date: May 20, 2021

(54) METHODS OF PREPARING AN ENRICHED SAMPLE FOR POLYPEPTIDE SEQUENCING

(71) Applicant: Quantum-Si Incorporated, Guilford, CT (US)

(72) Inventors: Brian Reed, Madison, CT (US); Matthew Dyer, Heber City, UT (US)

(73) Assignee: Quantum-Si Incorporated, Guilford, CT (US)

(21) Appl. No.: 17/082,899

(22) Filed: Oct. 28, 2020

Related U.S. Application Data
(60) Provisional application No. 62/926,897, filed on Oct. 28, 2019.

(51) Int. Cl. G01N 33/68; G01N 33/543

(52) U.S. Cl. CPC G01N 33/6818; G01N 33/54393

(57) ABSTRACT

Aspects of the application provide methods of preparing an enriched sample for polypeptide sequencing, and compositions, kits and devices useful for the same.

### RELATED APPLICATIONS

This application claims the benefit under 35 U.S.C. § 119(e) of the filing date of U.S. Provisional Application Ser. No. 62/926,897, filed Oct. 28, 2019, the entire contents of which is incorporated herein by reference.

### BACKGROUND OF INVENTION

Proteomics has emerged as an important and necessary complement to genomics and transcriptomics in the study of biological systems. Typically, only a fraction of the polypeptides in a complex sample is of interest (e.g., clinically relevant). However, the leveraging of molecules to enrich for polypeptides of interest in a highly multiplexed fashion has been limited to date.

### SUMMARY OF INVENTION

Provided herein are methods of preparing an enriched sample for polypeptide sequencing, which leverage enrichment molecules (and combinations of enrichment molecules) to increase the relative abundance of polypeptides of interest. These methods can be performed in a highly multiplexed fashion. Also provided herein are compositions, kits and devices useful for the same.

In some aspects, the disclosure relates to methods for preparing an enriched sample for polypeptide sequencing. In some embodiments, the method comprises: (i) using a plurality of enrichment molecules to select a subset of polypeptides from a plurality of polypeptides, thereby generating an enriched sample comprising the subset of polypeptides; and (ii) sequencing, in parallel, the polypeptides in the enriched sample. In some embodiments, the method comprises: (i) contacting a plurality of polypeptides with a plurality of enrichment molecules to produce an enriched sample comprising a subset of the polypeptides in the plurality of polypeptides; and (ii) sequencing, in parallel, the polypeptides of the enriched sample.

In some embodiments, (i) comprises: (a) contacting a plurality of polypeptides with a plurality of enrichment molecules, wherein at least a subset of the enrichment molecules in the plurality of enrichment molecules binds to a subset of the polypeptides in the plurality of polypeptides, thereby generating a bound subset of polypeptides and an unbound subset of polypeptides; and (b) isolating the bound subset of polypeptides to produce an enriched sample comprising a subset of the polypeptides in the plurality of polypeptides.

In some embodiments, (i) comprises: (a) contacting a plurality of polypeptides with a plurality of enrichment molecules, wherein at least a subset of the enrichment molecules in the plurality of enrichment molecules binds to a subset of the polypeptides in the plurality of polypeptides, thereby generating a bound subset of polypeptides and an unbound subset of polypeptides; and (b) isolating the unbound subset of polypeptides to produce an enriched sample comprising a subset of the polypeptides in the plurality of polypeptides.

In some embodiments, each of the enrichment molecules in the plurality of enrichment molecules comprises an antibody, an aptamer, or an enzyme. In some embodiments, the enrichment molecules in a subset of the plurality of enrichment molecules comprise an antibody, an aptamer, or an enzyme.

In some embodiments, each of the enrichment molecules in the plurality of enrichment molecules is immobilized on a substrate. In some embodiments, the enrichment molecules in a subset of the plurality of enrichment molecules are immobilized on a substrate. In some embodiments, the contacting of the plurality of polypeptides with the plurality of enrichment molecules in (i) occurs when a sample comprising the plurality of polypeptides contacts the substrate. In some embodiments, the substrate is selected from the group consisting of a surface, a bead, a particle, and a gel, optionally wherein: the surface is a solid surface; the bead is a magnetic bead; or the particle is a magnetic particle.

In some embodiments, each of the enrichment molecules in the plurality of enrichment molecules binds to two or more polypeptides comprising different amino acid sequences. In some embodiments, the enrichment molecules in a subset of the plurality of enrichment molecules bind to two or more polypeptides comprising different amino acid sequences.

In some embodiments, each of the enrichment molecules in the plurality of enrichment molecules binds to an amino acid post-translational modification. In some embodiments, the enrichment molecules in a subset of the plurality of enrichment molecules bind to an amino acid post-translational modification. In some embodiments, the post-translational modification is selected from the group consisting of acetylation, ADP-ribosylation, caspase cleavage, citrullination, formylation, hydroxylation, methylation, myristoylation, N-linked glycosylation, neddylation, nitration, O-linked glycosylation, oxidation, palmitoylation, phosphorylation, prenylation, S-nitrosylation, sulfation, sumoylation, ubiquitylation. In some embodiments, a first subset of the plurality of enrichment molecules bind to a first post-translational modification and a second subset of the enrichment molecules of the plurality of enrichment molecules bind to a second post-translational modification.

In some embodiments, (i) comprises: (a) contacting a plurality of polypeptides with a first plurality of enrichment molecules, wherein at least a subset of the enrichment molecules in the first plurality of enrichment molecules binds to a subset of the polypeptides in the plurality of polypeptides, thereby generating a first bound subset of polypeptides and a first unbound subset of polypeptides; (b) isolating the first bound subset of polypeptides or the first unbound subset of polypeptides of (a); and (c) iteratively repeating steps (a) and (b) with one or more additional plurality of enrichment molecules to produce an enriched sample comprising a subset of the polypeptides in the plurality of polypeptides. In some embodiments, the enrichment molecules in the first plurality bind to a post-translational modification and the enrichment molecules in a second plurality bind to one or more polypeptides of interest. In some embodiments, the enrichment molecules in the first plurality bind to one or more polypeptides of interest and the enrichment molecules in a second plurality bind to a post-translational modification.

In some embodiments, one or more of the polypeptides in the plurality of polypeptides is modified in vitro by contacting the polypeptides with a modifying agent prior to, concurrently with, or subsequently to the contacting of the plurality of polypeptides with the plurality of enrichment molecules in (i). In some embodiments, the modifying agent comprises a denaturant and at least one polypeptide is modified by denaturation. In some embodiments, the modifying agent blocks free carboxylate groups and at least one polypeptide is modified by blocking free carboxylate groups of the polypeptide. In some embodiments, the modifying agent blocks free thiol groups and at least one polypeptide is modified by blocking free thiol groups of the polypeptide. In some embodiments, the modifying agent comprises a cleaving agent and at least one polypeptide is modified by cleavage. In some embodiments, at least one polypeptide is modified by the addition of a post-translational modification. In some embodiments, the post-translational modification is selected from the group consisting of acetylation, ADP-ribosylation, caspase cleavage, citrullination, formylation, hydroxylation, methylation, myristoylation, N-linked glycosylation, neddylation, nitration, O-linked glycosylation, oxidation, palmitoylation, phosphorylation, prenylation, S-nitrosylation, sulfation, sumoylation, ubiquitylation.

In some embodiments, (ii) comprises: (a) contacting a single polypeptide molecule of the enriched sample with one or more terminal amino acid recognition molecules; and (b) detecting a series of signal pulses indicative of association of the one or more terminal amino acid recognition molecules with successive amino acids exposed at a terminus of the single polypeptide while the single polypeptide is being degraded, thereby sequencing the single polypeptide molecule.

In some embodiments, (ii) comprises: (a) contacting a single polypeptide molecule of the enriched sample with a composition comprising one or more terminal amino acid recognition molecules and a cleaving reagent; and (b) detecting a series of signal pulses indicative of association of the one or more terminal amino acid recognition molecules with a terminus of the single polypeptide molecule in the presence of the cleaving reagent, wherein the series of signal pulses is indicative of a series of amino acids exposed at the terminus over time as a result of terminal amino acid cleavage by the cleaving reagent.

In some embodiments, (ii) comprises: (a) identifying a first amino acid at a terminus of a single polypeptide molecule of the enriched sample; (b) removing the first amino acid to expose a second amino acid at the terminus of the single polypeptide molecule, and (c) identifying the second amino acid at the terminus of the single polypeptide molecule, wherein (a)-(c) are performed in a single reaction mixture.

In some embodiments, (ii) comprises: (a) contacting a single polypeptide molecule of the enriched sample with one or more amino acid recognition molecules that bind to the single polypeptide molecule; (b) detecting a series of signal pulses indicative of association of the one or more amino acid recognition molecules with the single polypeptide molecule under polypeptide degradation conditions; and (c) identifying a first type of amino acid in the single polypeptide molecule based on a first characteristic pattern in the series of signal pulses. In some embodiments, (ii) comprises: (a) obtaining data during a polypeptide degradation process; (b) analyzing the data to determine portions of the data corresponding to amino acids that are sequentially exposed at a terminus of the polypeptide during the degradation process; and (c) outputting an amino acid sequence representative of the polypeptide.

In some embodiments, (ii) comprises: (a) contacting a polypeptide of the enriched sample with one or more labeled affinity reagents that selectively bind one or more types of terminal amino acids at a terminus of the polypeptide; and (b) identifying a terminal amino acid at the terminus of the polypeptide by detecting an interaction of the polypeptide with the one or more labeled affinity reagents.

In some embodiments, (ii) comprises: (a) contacting a polypeptide in the enriched sample with one or more labeled affinity reagents that selectively bind one or more types of terminal amino acids at a terminus of the polypeptide; (b) identifying a terminal amino acid at the terminus of the polypeptide by detecting an interaction of the polypeptide with the one or more labeled affinity reagents; (c) removing the terminal amino acid; and (d) repeating (a)-(c) one or more times at the terminus of the polypeptide to determine an amino acid sequence of the polypeptide. In some embodiments, the method further comprises: after (a) and before (b), removing any of the one or more labeled affinity reagents that do not selectively bind the terminal amino acid; and/or after (b) and before (c), removing any of the one or more labeled affinity reagents that selectively bind the terminal amino acid. In some embodiments, (c) comprises modifying the terminal amino acid by contacting the terminal amino acid with an isothiocyanate, and: contacting the modified terminal amino acid with a protease that specifically binds and removes the modified terminal amino acid; or subjecting the modified terminal amino acid to acidic or basic conditions sufficient to remove the modified terminal amino acid. In some embodiments, identifying the terminal amino acid comprises: identifying the terminal amino acid as being one type of the one or more types of terminal amino acids to which the one or more labeled affinity reagents bind; or identifying the terminal amino acid as being a type other than the one or more types of terminal amino acids to which the one or more labeled affinity reagents bind.

In some embodiments, the one or more labeled affinity reagents comprise one or more labeled aptamers, one or more labeled peptidases, one or more labeled antibodies, one or more labeled degradation pathway protein, one or more aminotransferase, one or more tRNA synthetase, or a combination thereof. In some embodiments, the one or more labeled peptidases have been modified to inactivate cleavage activity; or wherein the one or more labeled peptidases retain cleavage activity for the removing of (c).

In some aspects, the disclosure relates to kits for use in performing the methods described herein. In some embodiments, the kit comprises a plurality of enrichment molecules. In some embodiments, each of the enrichment molecules in the plurality of enrichment molecules comprises an antibody, an aptamer, or an enzyme. In some embodiments, the enrichment molecules in a subset of the plurality of enrichment molecules comprise an antibody, an aptamer, or an enzyme.

In some embodiments, the kit comprises a modifying agent. In some embodiments, the modifying agent mediates polypeptide fragmentation, polypeptide denaturation, addition of a post-translational modification, and/or the blocking of one or more functional groups.

In some embodiments, the kit comprises a labeled affinity reagent. In some embodiments, the labeled affinity reagent comprises one or more labeled aptamers, one or more labeled peptidases, one or more labeled antibodies, one or more labeled degradation pathway protein, one or more aminotransferase, one or more tRNA synthetase, or a combination thereof.

In some aspects, the disclosure relates to a non-transitory computer-readable storage medium storing processor-executable instructions that, when executed by at least one hardware processor, cause the at least one hardware processor to perform a method of enrichment, as described herein.

In some aspects, the disclosure relates to devices for performing the methods described herein. In some embodiments, a device comprises: at least one hardware processor; and at least one non-transitory computer-readable storage medium storing processor-executable instructions that, when executed by the at least one hardware processor, cause the at least one hardware processor to perform a method of enrichment.

In some embodiments, the device comprises: (i) a sample preparation module configured to interface with one or more cartridge, each cartridge comprising: (a) one or more reservoirs or reaction vessels configured to receive a complex sample; (b) one or more sequence sample preparation reagents, wherein the sample preparation reagents comprise a plurality of enrichment molecules; and (c) a matrix comprising one or more immobilized capture probes; (ii) a sequencing module comprising an array of pixels, wherein each pixel is configured to receive a sequencing sample from the sample preparation module and comprises: (a) a sample well; and (b) at least one photodetector.

In some embodiments, at least a subset of the enrichment molecules in the plurality of enrichment molecules are immobilize on (e.g., covalently attached to) an immobilized capture probe. In some embodiments, at least a subset of the enrichment molecules is immobilized on (e.g., covalently attached to) a bead or particle that is capable of being bound by an immobilized capture probe.

In some embodiments, each of the enrichment molecules in the plurality of enrichment molecules comprises an antibody, an aptamer, or an enzyme. In some embodiments, the enrichment molecules in a subset of the plurality of enrichment molecules comprise an antibody, an aptamer, or an enzyme.

In some embodiments, the sample preparation reagents comprise a modifying agent. In some embodiments, the modifying agent mediates polypeptide fragmentation, polypeptide denaturation, addition of a post-translational modification, and/or the blocking of one or more functional groups.

In some embodiments, the sequencing module further comprises a reservoir or reaction vessel configured to deliver sequencing reagents to the sample well of each pixel.

In some embodiments, the sequencing reagents comprise a labeled affinity reagent. In some embodiments, the labeled affinity reagent comprises one or more labeled aptamers, one or more labeled peptidases, one or more labeled antibodies, one or more labeled degradation pathway protein, one or more aminotransferase, one or more tRNA synthetase, or a combination thereof.

### BRIEF DESCRIPTION OF THE DRAWINGS

The skilled artisan will understand that the figures, described herein, are for illustration purposes only. It is to be understood that, in some instances, various aspects of the invention may be shown exaggerated or enlarged to facilitate an understanding of the invention. In the drawings, like reference characters generally refer to like features, functionally similar and/or structurally similar elements throughout the various figures. The drawings are not necessarily to scale, emphasis instead being placed upon illustrating the principles of the teachings. The drawings are not intended to limit the scope of the present teachings in any way.

The features and advantages of the present invention will become more apparent from the detailed description set forth below when taken in conjunction with the drawings.

When describing embodiments in reference to the drawings, direction references (“above,” “below,” “top,” “bottom,” “left,” “right,” “horizontal,” “vertical,” etc.) may be used. Such references are intended merely as an aid to the reader viewing the drawings in a normal orientation. These directional references are not intended to describe the preferred or only orientation of an embodied device. A device may be embodied in other orientations.

As is apparent from the detailed description, the examples depicted in the figures and further described for the purpose of illustration throughout the application describe non-limiting embodiments, and in some cases may simplify certain processes or omit features or steps for the purpose of clearer illustration.

FIG. 1 provides an exemplary illustration of a complex sample. Complex samples are made up of many different polypeptides, only some of which may be of interest. A (square), B (rectangle), and C (circle) represent different polypeptides. Stars highlight polypeptides comprising a modification (e.g., a post-translational modification).

FIG. 2 provides exemplary illustrations of enrichment molecules. Enrichment molecules may bind to (or be bound by) one or more polypeptides. For example, enrichment molecule 1 binds to (or is bound by) polypeptide C (circle). In contrast, enrichment molecule 2 binds to (or is bound by) polypeptides A (square) and B (rectangle), and enrichment molecule 3 binds to (or is bound by) a polypeptide modification (here found on polypeptides A, B and C). In addition, enrichment molecules may be used in combinations.

FIG. 3 provides an exemplary embodiment of enrichment. Enrichment molecules are added to a complex mixture, which bind to (or are bound by) target polypeptides. Unbound polypeptides (here the polypeptides of interest) are then isolated, which are enriched relative to the target polypeptides. In this way, the relative abundance of the polypeptides of interest is increased.

FIG. 4 provides an exemplary embodiment of enrichment. Enrichment molecules are added to a complex mixture, which bind to (or are bound by) target polypeptides (here the polypeptides of interest). Bound polypeptides are then isolated, which are enriched relative to the non-target polypeptides. In this way, the relative abundance of the polypeptides of interest is increased.

FIG. 5 provides an illustration depicting an exemplary workflow of preparing an enriched sample.

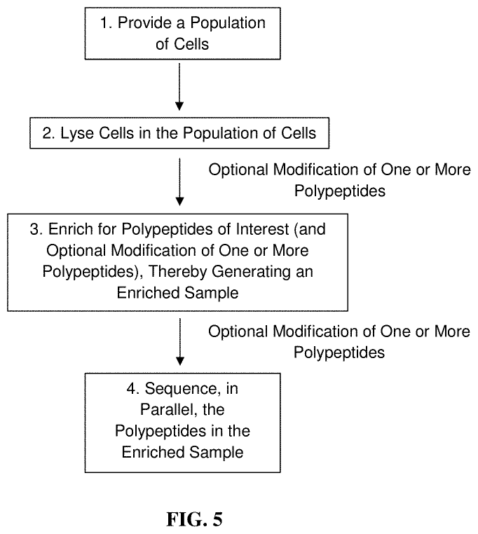

FIG. 6 provides an illustration depicting an exemplary workflow of preparing an enriched sample.

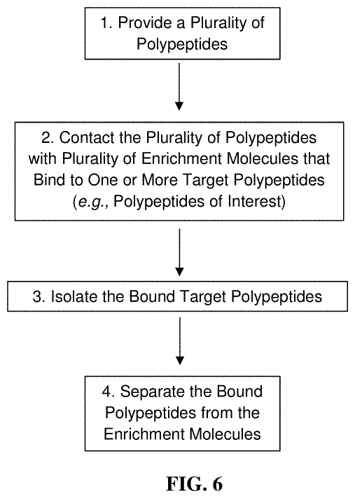

FIG. 7 provides an illustration depicting an exemplary workflow of preparing an enriched sample.

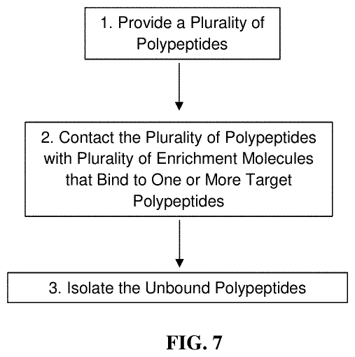

FIG. 8 provides an illustration depicting an exemplary apparatus for performing enrichment of a complex sample.

### DETAILED DESCRIPTION

As described herein, the inventors have recognized and appreciated that differential binding interactions can provide an additional or alternative approach to conventional labeling strategies in polypeptide sequencing. Conventional polypeptide sequencing can involve labeling each type of amino acid with a uniquely identifiable label. This process can be laborious and prone to error, as there are at least twenty different types of naturally occurring amino acids in addition to numerous post-translational variations thereof. In some aspects, the disclosure relates to the discovery of techniques involving the use of amino acid recognition molecules which differentially associate with different types of amino acids to produce detectable characteristic signatures indicative of an amino acid sequence of a polypeptide.

In some aspects, the disclosure relates to the discovery that a polypeptide sequencing reaction can be monitored in real-time using only a single reaction mixture (e.g., without requiring iterative reagent cycling through a reaction vessel). Conventional polypeptide sequencing reactions can involve exposing a polypeptide to different reagent mixtures to cycle between steps of amino acid detection and amino acid cleavage. Accordingly, in some aspects, the disclosure relates to an advancement in next generation sequencing that allows for the analysis of polypeptides by amino acid detection throughout an ongoing degradation reaction in real-time.

The proteomic analysis of an individual organism can provide insights into cellular processes and response patterns, which lead to improved diagnostic and therapeutic strategies. However, complex samples pose various challenges for proteomic analysis. Of particular relevance here, complex samples comprise a vast number of different polypeptides, and typically only a small fraction of the polypeptides is of interest (e.g., clinically relevant). For example, in a complex sample derived from blood, the vast majority of protein content (e.g., 99%) is made up of “house-keeping proteins,” such as albumin. As such, a significant portion of the data generated through proteomic analysis of complex samples is of little value. Without focusing the content of the data generated on an area of interest, important insights may be underappreciated and/or missed entirely. As described herein, the inventors have recognized and appreciated that the ability to enrich for polypeptides of interest would increase the efficiency of proteomic analysis of complex samples.

As such, in some aspects, the disclosure relates to methods of preparing an enriched sample for polypeptide sequencing (such as, by the methods disclosed herein), which leverage enrichment molecules (and combinations of enrichment molecules) to increase the relative abundance of polypeptides of interest. These methods can be performed in a highly multiplexed fashion, thereby increasing the efficiency of, and reducing the costs associated with, proteomic analysis of complex samples. Also provided herein are compositions, kits and devices useful for the same.

### I. Methods of Preparing a Complex Polypeptide Sample

In some aspects, the disclosure relates to methods of preparing a complex sample (e.g., a complex polypeptide sample). As used herein, the term “complex sample” refers to a sample comprising a plurality of molecules (e.g., polypeptides, polynucleic acids, metabolites, etc.), at least two of which are chemically unique. In some embodiments, a complex sample comprises a plurality of polypeptides, wherein the plurality comprises at least two polypeptides that comprise different amino acid sequences.

Typically, the complex sample is derived from a population of cells (e.g., produced by a population of cells). In some embodiments, the population of cells consists of a single cell. In other embodiments, the population of cells comprises two or more cells.

For example, in some embodiments the population of cells comprises at least 5, at least 10, at least 20, at least 30, at least 40, at least 50, at least 60, at least 70, at least 80, at least 90, at least 100, at least 150, at least 200, at least 250, at least 300, at least 350, at least 400, at least 450, a least 500, at least 600, at least 700, at least 800, at least 900, at least 1×10³, at least 1×10⁴, at least 1×10⁵, at least 1×10⁶, at least 1×10⁷, at least 1×10⁸, at least 1×10⁹, or at least 1×10¹⁰ cells.

In some embodiments, the population comprises 1-5, 1-10, 1-20, 1-30, 1-50, 1-60, 1-70, 1-80, 1-90, 1-100, 1-150, 1-200, 1-250, 1-300, 1-350, 1-400, 1-450, 1-500, 1-600, 1-700, 1-800, 1-900, 1-1×10³, 1-1×10⁴, 1-1×10⁵, 1-1×10⁶, 1-1×10⁷, 1-1×10⁸, 1-1×10⁹, 1-1×10¹⁰, 100-150, 100-200, 100-250, 100-300, 100-350, 100-400, 100-450, 100-500, 100-600, 100-700, 100-800, 100-900, 100-1×10³, 100-1×10⁴, 100-1×10⁵, 100-1×10⁶, 100-1×10⁷, 100-1×10⁸, 100-1×10⁹, 100-1×10¹⁰, 1×10³-1×10⁴, 1×10³-1×10⁵, 1×10³-1×10⁶, 1×10³-1×10⁷, 1×10³-1×10⁸, 1×10³-1×10⁹, 1×10³-1×10¹⁰, 1×10⁴-1×10⁵, 1×10⁴-1×10⁶, 1×10⁴-1×10⁷, 1×10⁴-1×10⁸, 1×10⁴-1×10⁹, 1×10⁴-1×10¹⁰, 1×10⁵-1×10⁶, 1×10⁵-1×10⁷, 1×10⁵-1×10⁸, 1×10⁵-1×10⁹, or 1×10⁵-1×10¹⁰ cells.

A population of cells may comprise prokaryotic cells and/or eukaryotic cells. A population of cells may comprise a plurality of homogeneous cells. Alternatively, a population of cells may comprise a plurality of heterogeneous cells.

A population of cells may be isolated from a subject (e.g., a multicellular or symbiotic organism). In some embodiments, the subject is a mouse, rat, rabbit, guinea pig, hamster, pig, sheep, dog, primate, cat, or human.

Methods of isolating populations of cells are known to those having skill in the art. For example, a method of preparing a complex sample may comprise biopsy, dissection (e.g., microdissection, such as laser capture), limited dilution, micromanipulation, immunomagnetic cell separation, fluorescence-activated cell sorting, density gradient centrifugation, immunodensity cell isolation, microfluidic cell sorting, sedimentation, adhesion, or a combination thereof.

In some embodiments, the method of preparing a complex sample comprises lysing a population of cells, thereby generating a lysis sample comprising a plurality of molecules (e.g., polypeptides, polynucleic acids, metabolites, etc.). Methods of lysing a population of cells are known to those having ordinary skill in the art. In some embodiments, a sample comprising cells is lysed using any one of known physical or chemical methodologies to release a target molecule from said cells. In some embodiments, a sample may be lysed using an electrolytic method, an enzymatic method, a detergent-based method, and/or mechanical homogenization. In some embodiments, if a sample does not comprise cells or tissue (e.g., a sample comprising purified polypeptides), a lysis step may be omitted.

Alternatively, or in addition, a method of preparing a complex sample may comprise subcellular fractionation (i.e., the isolation of one or more cellular compartment, such as endosomes, snyaptosomes, cytoplasm, nucleoplasm, chromatin, mitochondria, peroxisomes, lysosomes, melanosomes, exosomes, Golgi apparatus, endoplasmic reticulum, centrosomes, pseudopodia, or a combination thereof).

Molecules derived from the same cell population are described herein as having the same “origin.”

### II. Methods of Preparing an Enriched Sample

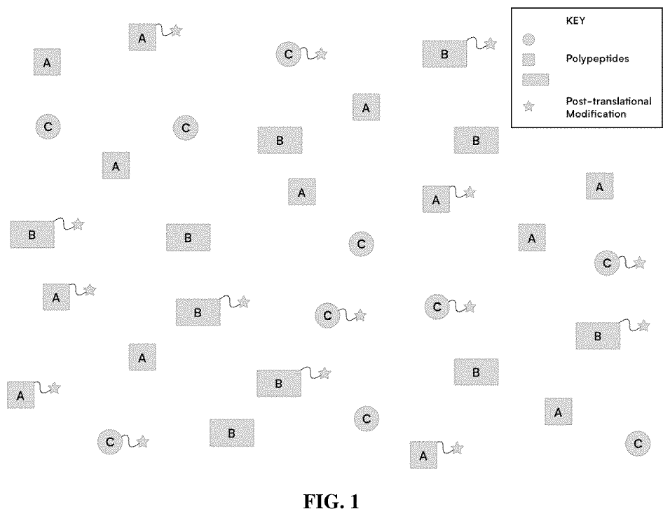

In some aspects, the disclosure relates to methods of enriching a sample for one or more molecules of interest (e.g., one or more polypeptide of interest). In particular, in some aspects, the disclosure relates to methods of polypeptide enrichment. As used herein, the term “polypeptide enrichment” refers to a process wherein the abundance of one or more polypeptides of interest is increased relative to the abundance of one or more reference polypeptides (e.g., a polypeptide in a complex sample that is not of interest). The term “polypeptide of interest” as used herein, refers to a polypeptide that one seeks to enrich. A polypeptide of interest may comprise a specific amino acid sequence. Alternatively, or in addition, a polypeptide of interest may comprise a specific polypeptide modification (e.g., a post-translational modification). These methods facilitate proteomic analysis of complex samples, which are made up of many different polypeptides, only some of which may be of interest (FIG. 1).

In some embodiments, a method for polypeptide enrichment comprises using a plurality of enrichment molecules to select a subset of polypeptides from a plurality of polypeptides, thereby generating an enriched sample comprising the subset of polypeptides. In some embodiments, the method comprises contacting a plurality of polypeptides with a plurality of enrichment molecules to produce an enriched sample comprising a subset of the polypeptides in the plurality of polypeptides.

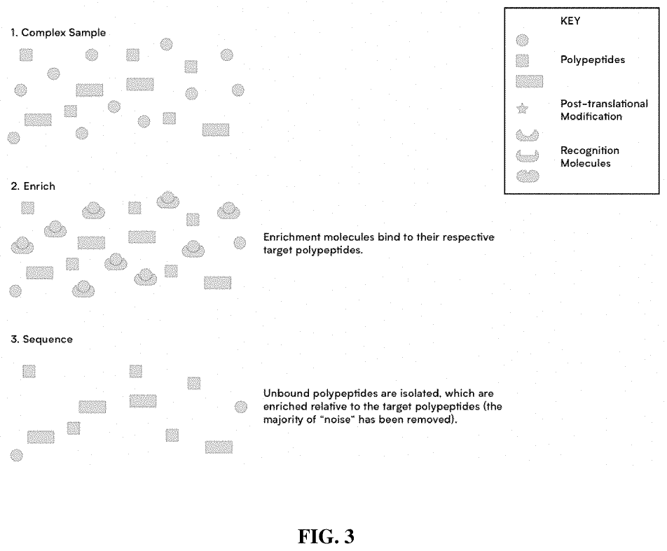

In some embodiments, a method for polypeptide enrichment comprises: (a) contacting a plurality of polypeptides with a plurality of enrichment molecules, wherein at least a subset of the enrichment molecules in the plurality of enrichment molecules binds to a subset of the polypeptides in the plurality of polypeptides, thereby generating a bound subset of polypeptides and an unbound subset of polypeptides; and (b) isolating the bound subset of polypeptides to produce an enriched sample comprising a subset of the polypeptides in the plurality of polypeptides. The polypeptide enrichment methodology illustrated in FIG. 3 provides an example of this embodiment.

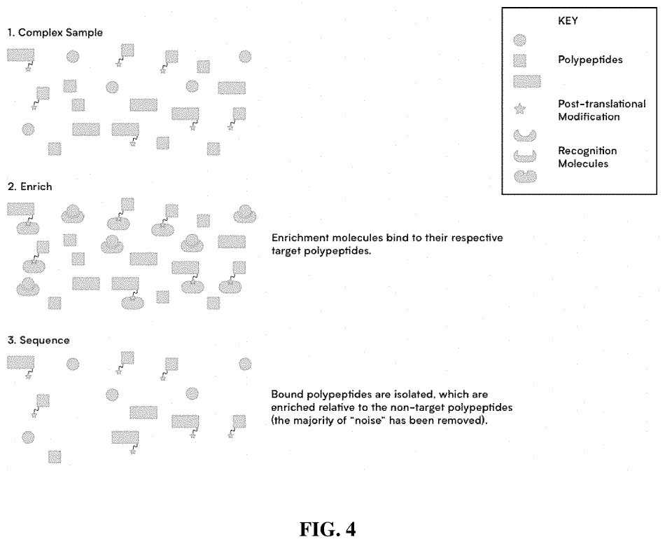

In some embodiments, a method for polypeptide enrichment comprises: (a) contacting a plurality of polypeptides with a plurality of enrichment molecules, wherein at least a subset of the enrichment molecules in the plurality of enrichment molecules binds to a subset of the polypeptides in the plurality of polypeptides, thereby generating a bound subset of polypeptides and an unbound subset of polypeptides; and (b) isolating the unbound subset of polypeptides to produce an enriched sample comprising a subset of the polypeptides in the plurality of polypeptides. The polypeptide enrichment methodology illustrated in FIG. 4 provides an example of this embodiment.

In the embodiments described in the preceding paragraphs, it is understood that the binding of an enrichment molecule to a polypeptide is equivalent to the binding of the polypeptide to the enrichment molecule. Accordingly, step (a) in the embodiments described above can be equivalently describe as: (a) contacting a plurality of polypeptides with a plurality of enrichment molecules, wherein at least a subset of the enrichment molecules in the plurality of enrichment molecules is bound by a subset of the polypeptides in the plurality of polypeptides, thereby generating a bound subset of polypeptides and an unbound subset of polypeptides.

It is also understood that steps (a) and (b) of the embodiments described above may be repeated one or more times using additional pluralities of enrichment molecules to produce a further enriched sample. For example, in some embodiments, the method comprises: (a) contacting a plurality of polypeptides with a first plurality of enrichment molecules, wherein at least a subset of the enrichment molecules in the first plurality of enrichment molecules binds to a subset of the polypeptides in the plurality of polypeptides, thereby generating a first bound subset of polypeptides and a first unbound subset of polypeptides; (b) isolating the first bound subset of polypeptides or the first unbound subset of polypeptides of (a); and (c) iteratively repeating steps (a) and (b) with one or more additional plurality of enrichment molecules to produce an enriched sample comprising a subset of the polypeptides in the plurality of polypeptides. In some embodiments, steps (a) and (b) are repeated using a second, third, fourth, fifth, sixth, seventh, eighth, ninth, tenth, or any number of additional plurality of enrichment molecules.

For example, in some embodiments the method comprises: (a) contacting a plurality of polypeptides with a first plurality of enrichment molecules, wherein at least a subset of the enrichment molecules in the first plurality of enrichment molecules binds to a subset of the polypeptides in the plurality of polypeptides, thereby generating a first bound subset of polypeptides and a first unbound subset of polypeptides; (b) isolating the first bound subset of polypeptides or the first unbound subset of polypeptides of (a); (c) contacting the isolated polypeptides of (b) with a second plurality of enrichment molecules, wherein at least a subset of the enrichment molecules in the second plurality of enrichment molecules binds to a subset of the polypeptides isolated in (b), thereby generating a second bound subset of polypeptides and a second unbound subset of polypeptides; (d) isolating the second bound subset of polypeptides or the second unbound subset of polypeptides of (c) to produce an enriched sample comprising a subset of the polypeptides in the plurality of polypeptides.

Alternatively, or in addition, a method of enrichment may comprise chromatography (e.g., size exclusion, ion exchange, etc.), isoelectric focusing, membrane filtration, molecular sieve filtration, concentration, precipitation (e.g., cryoprecipitation), dry down, dialysis, or a combination thereof.

In some embodiments, the method comprises contacting a complex sample with a kit or device described herein. See “Kits for Sample Preparation” and “Devices for Sample Preparation and Sample Sequencing”.

In some embodiments, the polypeptides in an enriched sample are identical (i.e., contain the same amino acid sequence). In some embodiments, an enriched sample comprises at least two unique polypeptides (i.e., having differing amino acid sequences). For example, in some embodiments, an enriched sample comprises at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 25, at least 30, at least 40, at least 50, at least 60, at least 70, at least 80, at least 90, or at least 100 unique polypeptides. In some embodiments, an enriched sample comprises 1-2, 1-5, 1-10, 1-15, 1-20, 1-30, 1-40, 1-50, 1-60, 1-70, 1-80, 1-90, 1-100, 2-5, 2-10, 2-15, 2-20, 2-30, 2-40, 2-50, 2-60, 2-70, 2-80, 2-90, 2-100, 5-10, 5-15, 5-20, 5-30, 5-40, 5-50, 5-60, 5-70, 5-80, 5-90, 5-100, 10-15, 10-20, 10-30, 10-40, 10-50, 10-60, 10-70, 10-80, 10-90, 10-100, 15-20, 20-30, 20-40, 20-50, 20-60, 20-70, 20-80, 20-90, 20-100, 20-30, 20-40, 20-50, 20-60, 20-70, 20-80, 20-90, 20-100, 30-40, 30-50, 30-60, 30-70, 30-80, 30-90, 30-100, 40-50, 40-60, 40-70, 40-80, 40-90, 40-100, 50-60, 50-70, 50-80, 50-90, or 50-100 unique polypeptides.

In some embodiments, the enriched sample comprises polypeptides that share at least 50%, 60%, 70%, 80%, 90% 95%, or 99% sequence identity. In some embodiments, the enriched sample comprises polypeptides that share one or more polypeptide modification (e.g., post-translational modification). Examples of post-translational modifications are known to those having skill in the art and include, but are not limited to, acetylation, adenylylation, ADP-ribosylation, alkylation (e.g., methylation), amidation, arginylation, biotinylation, butyrylation, carbamylation, carbonylation, carboxylation, citrullination, deamidation, eliminylation, formylation, glycosylation (e.g., N-linked glycosylation, O-linked glycosylation), glipyatyon, glycation, hydroxylation, iodination, ISGylation, isoprenylation, lipoylation, malonylation, myristoylation, neddylation, nitration, oxidation, palmitoylation pegylation, phosphorylation, phosphopantetheinylation, polyglcylation, polyglutamylation, prenylation, propionylation, pupylation, S-glutathionylation, S-nitrosylation, S-sulfenylation, S-sulfinylation, S-sulfonylation, succinylation, sulfation, SUMOylation, and ubiquitination.

### A. Enrichment Molecules

As used herein, the term “enrichment molecule” refers to a molecule that exhibits preferentially binding to (or by) one or more target polypeptides. An enrichment molecule may bind to (or be bound by) a target polypeptide through a direct interaction with the amino acid sequence of the target polypeptide. Alternatively, or in addition, an enrichment molecule may bind to (or be bound by) a target polypeptide through an interaction with a modification of the target polypeptide (e.g., a post-translational modification). The binding of an enrichment molecule to (or by) a target polypeptide may be mediated through electrostatic interactions, hydrophobic interactions, complementary shape, or a combination thereof.

In some embodiments, a target polypeptide is a polypeptide of interest. In other embodiments, a target polypeptide is not a polypeptide of interest.

Exemplary enrichment molecules that preferentially bind to one or more target polypeptides (or target polypeptide variants) include immunoglobulins, anticalins, lipocalins, DARPins, aptamers, enzymes, lectins, and peptide interaction domains.

As used herein, the term “immunoglobulin” refers to polypeptides characterized as having an immunoglobulin fold and which function as antibodies and bind to one or more substrates (e.g., target polypeptides). As such, the term “immunoglobulin” encompasses conventional immunoglobulins (i.e., IgA, IgD, IgE, IgG, and IgM), single-chain variable fragments (scFv), antigen-binding fragments (Fab), affibodies, and single domain antibodies (sdAb), such as Nanobodies, VHHs and VNARs.

The term “aptamer” as used herein refers to a polynucleic acid (e.g., DNA or RNA) or polypeptide that preferentially binds to one or more target molecules (e.g., target polypeptides). Although there are examples found in nature, aptamers are usually engineered through repeated rounds of in vitro selection.

As used herein, the term “enzyme” refers to a macromolecular biological catalyst that accelerates a chemical reaction upon binding one or more substrates (e.g., target polypeptides). Typically, an enzyme will release its substrate after completion of a chemical reaction. As such, in some embodiments, wherein an enrichment molecule comprises an enzyme, the enzyme is catalytically inactivated so as to increase the likelihood that the enzyme remains bound to the substrate. Catalytic inactivation may be performed via mutagenesis and/or depletion of one or more enzymatic cofactor (i.e., a non-protein chemical compound or metallic ion that is required for an enzyme's activity as a catalyst).

The term “peptide interaction domain” as used herein, refers to a polypeptide (or a portion of a polypeptide) that interacts with one or more polypeptides (e.g., target polypeptides).

For example, a peptide interaction domain may be a scaffold protein, a polypeptide of a multiprotein complex, or a portion thereof.

In some embodiments, an enrichment molecule comprises an immunoglobulin, an aptamer, an enzyme, and/or a peptide interaction domain.

Exemplary enrichment molecules that are preferentially bound by one or more target polypeptides include oligonucleotides (e.g., double-stranded DNA, single-stranded DNA, double-stranded RNA, single-stranded RNA, or the like), oligosaccharides (or polysaccharides), lipids, glycoproteins, receptor ligands, receptor agonists, receptor antagonists, enzyme substrates, and enzyme cofactors.

In some embodiments, an enrichment molecule comprises an oligonucleotide (e.g., double-stranded DNA, single-stranded DNA, double-stranded RNA, single-stranded RNA, or the like), an oligosaccharide, a lipid, a receptor ligand, a receptor agonist, a receptor antagonist, an enzyme substrate, and/or an enzyme cofactor.

Preferential binding is used herein to characterize enrichment molecules to emphasize: (i) that an enrichment molecule need not exhibit high specificity (i.e., only bind to (or be bound by) a single target polypeptide to an appreciable level); (ii) that an enrichment molecule may exhibit some degree of off-target binding (i.e., bind to (or be bound by) an off-target molecule to a detectable level); and (iii) that an enrichment molecule need not bind to a target polypeptide with 100% efficiency (i.e., not all target polypeptides in a complex sample need necessarily be bound, even in the presence of excess enrichment molecules).

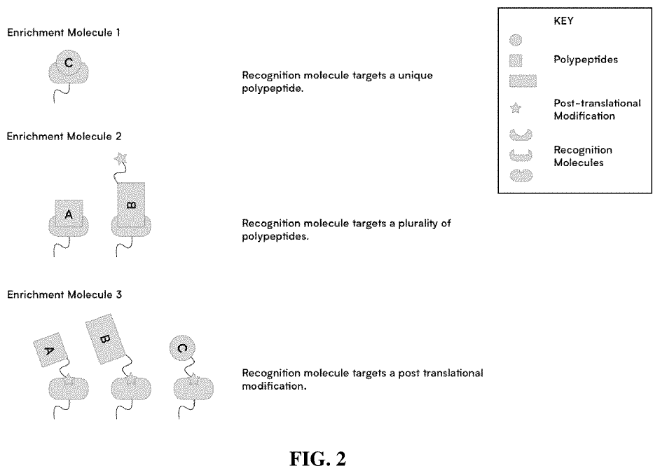

In some embodiments, an enrichment molecule preferentially binds to (or is preferentially bound by) a single target polypeptide (e.g., enrichment molecule 1 of FIG. 2). However, in other embodiments, an enrichment molecule preferential binds to (or is preferentially bound by) two or more target polypeptides (e.g., enrichment molecules 2 and 3 of FIG. 2).

In some embodiments, an enrichment molecule exhibits preferential binding to (or is preferentially bound by) at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 25, at least 30, at least 40, at least 50, at least 60, at least 70, at least 80, at least 90, or at least 100, at least 200, at least 300, at least 400, at least 500, at least 600, at least 700, at least 800, at least 900, at least 1000, at least 2000, at least 3000, at least 4000, at least 5000, or at least 10,000 target polypeptides.

In some embodiments, an enrichment molecule exhibits preferential binding to (or is preferentially bound by) two, three, four, five, six, seven, eight, nine, ten, eleven, twelve, thirteen, fourteen, or fifteen target polypeptides.

In some embodiments, an enrichment molecule exhibits preferential binding to (or is preferentially bound by) 1-2, 1-5, 1-10, 1-15, 1-20, 1-30, 1-40, 1-50, 1-60, 1-70, 1-80, 1-90, 1-100, 2-5, 2-10, 2-15, 2-20, 2-30, 2-40, 2-50, 2-60, 2-70, 2-80, 2-90, 2-100, 5-10, 5-15, 5-20, 5-30, 5-40, 5-50, 5-60, 5-70, 5-80, 5-90, 5-100, 10-15, 10-20, 10-30, 10-40, 10-50, 10-60, 10-70, 10-80, 10-90, 10-100, 15-20, 20-30, 20-40, 20-50, 20-60, 20-70, 20-80, 20-90, 20-100, 20-30, 20-40, 20-50, 20-60, 20-70, 20-80, 20-90, 20-100, 30-40, 30-50, 30-60, 30-70, 30-80, 30-90, 30-100, 40-50, 40-60, 40-70, 40-80, 40-90, 40-100, 50-60, 50-70, 50-80, 50-90, or 50-100, 100-200, 100-300, 100-400, 100-500, 100-600, 100-700, 100-800, 100-900, 100-1000, 100-5000, 100-10,000, 500-600, 500-700, 500-800, 500-900, 500-1000, 500-5000, 500-10,000, 1000-5000, or 1000-10,000 target polypeptides.

In some embodiments, an enrichment molecule exhibits preferential binding to (or is preferentially bound by) a plurality of related target polypeptides (e.g., 2, 3, 4, 5, 6, 7, 8, 9, 10, 20, 30, 40, 50, or more related polypeptides) that share at least 50%, 60%, 70%, 80%, 90% 95%, or 99% sequence homology.

In some embodiments, an enrichment molecule exhibits preferential binding to (or is preferentially bound by) a post-translational modification, such as acetylation, adenylylation, ADP-ribosylation, alkylation (e.g., methylation), amidation, arginylation, biotinylation, butyrylation, carbamylation, carbonylation, carboxylation, citrullination, deamidation, eliminylation, formylation, glycosylation (e.g., N-linked glycosylation, O-linked glycosylation), glipyatyon, glycation, hydroxylation, iodination, ISGylation, isoprenylation, lipoylation, malonylation, myristoylation, neddylation, nitration, oxidation, palmitoylation pegylation, phosphorylation, phosphopantetheinylation, polyglcylation, polyglutamylation, prenylation, propionylation, pupylation, S-glutathionylation, S-nitrosylation, S-sulfenylation, S-sulfinylation, S-sulfonylation, succinylation, sulfation, SUMOylation, and ubiquitination

An enrichment molecule may be immobilized on (e.g., covalently attached to) a substrate (e.g., a capture probe as described in “Devices for Sample Preparation and Sample Sequencing”). The substrate may be a surface (e.g., a solid surface), a bead (e.g., a magnetic bead), a particle (e.g., a magnetic particle), or a gel.

### (i) Pluralities of Enrichment Molecules

Typically, the enrichment methods described herein utilize a plurality of enrichment molecules. The enrichment molecules in a plurality may be chemically identical (i.e., a plurality having one enrichment molecule “type”). Alternatively, pluralities of enrichment molecules may contain a combination of different enrichment molecules (i.e., have two or more enrichment molecule “types”).

In some embodiments, a plurality of enrichment molecules contains a single enrichment molecule type. In other embodiments, a plurality of enrichment molecules comprises a combination of two or more, three or more, four or more, five or more, six or more, seven or more, eight or more, nine or more, ten or more, eleven or more, twelve or more, thirteen or more, fourteen or more, or fifteen or more enrichment molecule types. In some embodiments, a plurality of enrichment molecules comprises at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 25, at least 30, at least 40, at least 50, at least 60, at least 70, at least 80, at least 90, or at least 100, at least 200, at least 300, at least 400, at least 500 enrichment molecule types.

In some embodiments, a plurality of enrichment molecules comprises a combination of two, three, four, five, six, seven, eight, nine, ten, eleven, twelve, thirteen, fourteen, or fifteen enrichment molecule types.

In some embodiments, a plurality of enrichment molecules contains a combination of 1-2, 1-5, 1-10, 1-15, 1-20, 1-30, 1-40, 1-50, 1-60, 1-70, 1-80, 1-90, 1-100, 2-5, 2-10, 2-15, 2-20, 2-30, 2-40, 2-50, 2-60, 2-70, 2-80, 2-90, 2-100, 5-10, 5-15, 5-20, 5-30, 5-40, 5-50, 5-60, 5-70, 5-80, 5-90, 5-100, 10-15, 10-20, 10-30, 10-40, 10-50, 10-60, 10-70, 10-80, 10-90, 10-100, 15-20, 20-30, 20-40, 20-50, 20-60, 20-70, 20-80, 20-90, 20-100, 20-30, 20-40, 20-50, 20-60, 20-70, 20-80, 20-90, 20-100, 30-40, 30-50, 30-60, 30-70, 30-80, 30-90, 30-100, 40-50, 40-60, 40-70, 40-80, 40-90, 40-100, 50-60, 50-70, 50-80, 50-90, or 50-100, 100-200, 100-300, 100-400, or 100-500 enrichment molecule types.

In some embodiments, each of the enrichment molecules in the plurality of enrichment molecules preferentially binds to (or is preferentially bound by) a single target polypeptide. In other embodiments, one or more (e.g., a subset) of the enrichment molecules in a plurality of enrichment molecules exhibits preferential binding to (or is preferentially bound by) two or more target polypeptides. In yet other embodiments, each of the enrichment molecules in the plurality of enrichment molecules exhibits preferential binding to (or is preferentially bound by) two or more target polypeptides.

In some embodiments, one or more (e.g., a subset) of the enrichment molecules in the plurality of enrichment molecules binds to a post-translational polypeptide modification. In other embodiments, each of the enrichment molecules in a plurality of enrichment molecules exhibits preferential binding to two or more post-translational polypeptide modifications.

In some embodiments, each of the enrichment molecules in the plurality of enrichment molecules is immobilized on (e.g., covalently attached to) a substrate (e.g., a capture probe as described in “Devices for Sample Preparation and Sample Sequencing”), such as a surface (e.g., a solid surface), a bead (e.g., a magnetic bead), a particle (e.g., a magnetic particle, or a gel). In some embodiments, one or more (e.g., a subset) of the plurality of enrichment molecules is immobilized on (e.g., covalently attached to) a substrate. As such, in some embodiments, the contacting of the plurality of polypeptides with the plurality of enrichment molecules occurs when a sample comprising the plurality of polypeptides contacts the substrate.

For example, in some embodiments, the enrichment molecules are immobilized on (e.g., covalently attached or crosslinked to) a gel and the sample is pulled through the gel. In some embodiments, the enrichment molecules are immobilized on (e.g., covalently attached to) a bead (e.g., a magnetic bead), which are then pulled down.

### (ii) Multiple Enrichment Molecule Pluralities

As described above, in some embodiments, the method comprises: (a) contacting a plurality of polypeptides with a first plurality of enrichment molecules, wherein at least a subset of the enrichment molecules in the first plurality of enrichment molecules binds to a subset of the polypeptides in the plurality of polypeptides, thereby generating a first bound subset of polypeptides and a first unbound subset of polypeptides; (b) isolating the first bound subset of polypeptides or the first unbound subset of polypeptides of (a); and (c) iteratively repeating steps (a) and (b) with one or more additional plurality of enrichment molecules to produce an enriched sample comprising a subset of the polypeptides in the plurality of polypeptides. In some embodiments, steps (a) and (b) are repeated using a second, third, fourth, fifth, sixth, seventh, eighth, ninth, tenth, or any number of additional plurality of enrichment molecules.

In some embodiments, each plurality of enrichment molecules utilized in the method of polypeptide enrichment is unique (i.e., each comprises a different plurality of enrichment molecules). In other embodiments, two or more of the pluralities are identical. In some embodiments, at least one of the pluralities of enrichment molecules targets a post-translational polypeptide modification and at least one of the pluralities of enrichment molecules does not target a post-translational modification.

For example, the first enrichment step (utilizing a first plurality of enrichment molecules) may enrich of a particular post-translational polypeptide modification, and a second enrichment step (utilizing a second plurality of enrichment molecules) may enrich for a particular polypeptide (and variants of that polypeptide). Alternatively, the first enrichment step (utilizing a first plurality of enrichment molecules) may enrich of a particular polypeptide (and variants of that polypeptide), and a second enrichment step (utilizing a second plurality of enrichment molecules) may enrich for a particular post-translational modification.

### B. Polypeptide Modifications

One or more of the polypeptides of a complex sample may be modified in vitro prior to, concurrently with, and/or subsequent to the polypeptide enrichment described above. For example, in some embodiments, a complex sample is contacted with a modifying agent prior to, concurrently with, and/or subsequent to performance of polypeptide enrichment. Among other things, a modifying agent may mediate polypeptide fragmentation, polypeptide denaturation, addition of a post-translational modification, and/or the blocking of one or more functional groups.

In some embodiments, one or more polypeptides of a complex sample are modified by fragmentation. In some embodiments, fragmentation comprises enzymatic digestion. In some embodiments, digestion is carried out by contacting a polypeptide with an endopeptidase (e.g., trypsin) under digestion conditions. In some embodiments, fragmentation comprises chemical digestion. Examples of suitable reagents for chemical and enzymatic digestion are known in the art and include, without limitation, trypsin, chemotrypsin, Lys-C, Arg-C, Asp-N, Lys-N, BNPS-Skatole, CNBr, caspase, formic acid, glutamyl endopeptidase, hydroxylamine, iodosobenzoic acid, neutrophil elastase, pepsin, proline-endopeptidase, proteinase K, staphylococcal peptidase I, thermolysin, and thrombin.

In some embodiments, one or more polypeptides of a complex sample are modified by denaturation (e.g., by heat and/or chemical means).

In some embodiments, one or more polypeptides of a complex sample are modified by in vitro post-translational modification, such as by acetylation, adenylylation, ADP-ribosylation, alkylation (e.g., methylation), amidation, arginylation, biotinylation, butyrylation, carbamylation, carbonylation, carboxylation, citrullination, deamidation, eliminylation, formylation, glycosylation (e.g., N-linked glycosylation, O-linked glycosylation), glipyatyon, glycation, hydroxylation, iodination, ISGylation, isoprenylation, lipoylation, malonylation, myristoylation, neddylation, nitration, oxidation, palmitoylation pegylation, phosphorylation, phosphopantetheinylation, polyglcylation, polyglutamylation, prenylation, propionylation, pupylation, S-glutathionylation, S-nitrosylation, S-sulfenylation, S-sulfinylation, S-sulfonylation, succinylation, sulfation, SUMOylation, or ubiquitination.

In some embodiments, one or more polypeptides of a complex sample are modified by the blocking of one or more functional groups (e.g., free carboxylate groups and/or thiol groups).

In some embodiments, blocking free carboxylate groups refers to a chemical modification of these groups which alters chemical reactivity relative to an unmodified carboxylate. Suitable carboxylate blocking methods are known in the art and should modify side-chain carboxylate groups to be chemically different from a carboxy-terminal carboxylate group of a polypeptide to be functionalized. In some embodiments, blocking free carboxylate groups comprises esterification or amidation of free carboxylate groups of a polypeptide. In some embodiments, blocking free carboxylate groups comprises methyl esterification of free carboxylate groups of a polypeptide, e.g., by reacting the polypeptide with methanolic HCl. Additional examples of reagents and techniques useful for blocking free carboxylate groups include, without limitation, 4-sulfo-2,3,5,6-tetrafluorophenol (STP) and/or a carbodiimide such as N-(3-Dimethylaminopropyl)-N′-ethylcarbodiimide hydrochloride (EDAC), uronium reagents, diazomethane, alcohols and acid for Fischer esterification, the use of N-hydroxylsuccinimide (NHS) to form NHS esters (potentially as an intermediate to subsequent ester or amine formation), or reaction with carbonyldiimidazole (CDI) or the formation of mixed anhydrides, or any other method of modifying or blocking carboxylic acids, potentially through the formation of either esters or amides.

In some embodiments, blocking free thiol groups refers to a chemical modification of these groups which alters chemical reactivity relative to an unmodified thiol. In some embodiments, blocking free thiol groups comprises reducing and alkylating free thiol groups of a polypeptide. In some embodiments, reduction and alkylation is carried out by contacting a polypeptide with dithiothreitol (DTT) and one or both of iodoacetamide and iodoacetic acid. Examples of additional and alternative cysteine-reducing reagents which may be used are well known and include, without limitation, 2-mercaptoethanol, Tris (2-carboxyehtyl) phosphine hydrochloride (TCEP), tributylphosphine, dithiobutylamine (DTBA), or any reagent capable of reducing a thiol group. Examples of additional and alternative cysteine-blocking (e.g., cysteine-alkylating) reagents which may be used are well known and include, without limitation, acrylamide, 4-vinylpyridine, N-Ethylmalemide (NEM), N-ε-maleimidocaproic acid (EMCA), or any reagent that modifies cysteines so as to prevent disulfide bond formation.

In some embodiments, the N-terminal amino acid or the C-terminal amino acid of a polypeptide is modified.

In some embodiments, a carboxy-terminus of a polypeptide is modified in a method comprising: (i) blocking free carboxylate groups of the polypeptide; (ii) denaturing the polypeptide (e.g., by heat and/or chemical means); (iii) blocking free thiol groups of the polypeptide; (iv) digesting the polypeptide to produce at least one polypeptide fragment comprising a free C-terminal carboxylate group; and (v) conjugating (e.g., chemically) a functional moiety to the free C-terminal carboxylate group. In some embodiments, the method further comprises, after (i) and before (ii), dialyzing a sample comprising the polypeptide.

In some embodiments, a carboxy-terminus of a polypeptide is modified in a method comprising: (i) denaturing the polypeptide (e.g., by heat and/or chemical means); (ii) blocking free thiol groups of the polypeptide; (iii) digesting the polypeptide to produce at least one polypeptide fragment comprising a free C-terminal carboxylate group; (iv) blocking the free C-terminal carboxylate group to produce at least one polypeptide fragment comprising a blocked C-terminal carboxylate group; and (v) conjugating (e.g., enzymatically) a functional moiety to the blocked C-terminal carboxylate group. In some embodiments, the method further comprises, after (iv) and before (v), dialyzing a sample comprising the polypeptide.

In some embodiments, a complex sample is contacted with a modifying agent prior to enrichment to mediate polypeptide fragmentation, polypeptide denaturation, addition of a post-translational modification, and/or the blocking of one or more functional groups. Alternatively, or in addition, in some embodiments, a complex sample with a modifying agent concurrently with enrichment to mediate polypeptide fragmentation, polypeptide denaturation, addition of a post-translational modification, and/or the blocking of one or more functional groups. Alternatively, or in addition, in some embodiments, a complex sample (or a sample derived therefrom, comprising the one or more polypeptides of interest) with a modifying agent after enrichment to mediate polypeptide fragmentation, polypeptide denaturation, addition of a post-translational modification, and/or the blocking of one or more functional groups.

### III. Polypeptide Sequencing Methodologies

In some embodiments, polypeptides of an enriched sample are sequenced and/or identified following enrichment. As such, in some aspects, the disclosure relates to methods of polypeptide sequencing and identification. Various methods of sequencing polypeptide molecules are known to those having ordinary skill in the art and include mass spectrometry (e.g., peptide mass fingerprinting and tandem mass spectrometry) and Edman degradation. Additional, previously undescribed methods of sequencing polypeptides are described herein.

As used herein, “sequencing,” “sequence determination,” “determining a sequence,” and like terms, in reference to a polypeptide include determination of partial amino acid sequence information as well as full amino acid sequence information of the polypeptide. That is, the terminology includes sequence comparisons, fingerprinting, and like levels of information about a target molecule, as well as the express identification and ordering of each amino acid of the target molecule within a region of interest. The terminology includes identifying a single amino acid (or the probability of a single amino acid) of a polypeptide. In some embodiments, more than one amino acid (or the probability of more than one amino acid) of a polypeptide is identified. Accordingly, in some embodiments, the terms “amino acid sequence” and “polypeptide sequence” as used herein may refer to the polypeptide material itself and is not restricted to the specific sequence information (e.g., the succession of letters representing the order of amino acids from one terminus to another terminus) that biochemically characterizes a specific polypeptide.

In some embodiments, the probability of an amino acid at a specific position within a polypeptide is determined and illustrated in a probability array. For example, for a polypeptide consisting of two amino acids, the terms “sequencing,” “sequence determination,” “determining a sequence,” and like terms may involve determining the probability of an amino at position 1 and/or position 2, such as [[0.80, 0.12, 0.05, 0.01, 0.01, 0.01, 0.00, 0.00, 0.00, 0.00, 0.00, 0.00, 0.00, 0.00, 0.00, 0.00, 0.00, 0.00, 0.00, 0.00], [0.00, 0.10, 0.90, 0.00, 0.00, 0.00, 0.00, 0.00, 0.00, 0.00, 0.00, 0.00, 0.00, 0.00, 0.00, 0.00, 0.00, 0.00, 0.00, 0.00]] where the probabilities in the array correspond to A, R, N, D, C, Q, E, G, H, I, L, K, M, F, P, S, T, W, Y, and V, respectively. One having ordinary skill in the art will understand that this example (and exemplary probability array) can be expanded to accommodate the analysis of additional amino acid identities (e.g., modified amino acids), such as those described herein.

In some embodiments, sequencing of a polypeptide molecule comprises identifying at least two (e.g., at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 25, at least 30, at least 35, at least 40, at least 45, at least 50, at least 60, at least 70, at least 80, at least 90, at least 100, or more) amino acids (or amino acid probabilities) in the polypeptide molecule. In some embodiments, the at least two amino acids are contiguous amino acids. In some embodiments, the at least two amino acids are non-contiguous amino acids.

In some embodiments, sequencing of a polypeptide molecule comprises identification of less than 100% (e.g., less than 99%, less than 95%, less than 90%, less than 85%, less than 80%, less than 75%, less than 70%, less than 65%, less than 60%, less than 55%, less than 50%, less than 45%, less than 40%, less than 35%, less than 30%, less than 25%, less than 20%, less than 15%, less than 10%, less than 5%, less than 1% or less) of all amino acids in the polypeptide molecule. For example, in some embodiments, sequencing of a polypeptide molecule comprises identification of less than 100% of one type of amino acid in the polypeptide molecule (e.g., identification of a portion of all amino acids of one type in the polypeptide molecule). In some embodiments, sequencing of a polypeptide molecule comprises identification of less than 100% of each type of amino acid in the polypeptide molecule.

In some embodiments, sequencing of a polypeptide molecule comprises identification of at least 1, at least 5, at least 10, at least 15, at least 20, at least 25, at least 30, at least 35, at least 40, at least 45, at least 50, at least 55, at least 60, at least 65, at least 70, at least 75, at least 80, at least 85, at least 90, at least 95, at least 100 or more types of amino acids in the polypeptide.

In some embodiments, the application provides compositions and methods for sequencing a polypeptide by identifying a series of amino acids that are present at a terminus of a polypeptide over time (e.g., by iterative detection and cleavage of amino acids at the terminus). In yet other embodiments, the application provides compositions and methods for sequencing a polypeptide by identifying labeled amino content of the polypeptide and comparing to a reference sequence database.

In some embodiments, the application provides compositions and methods for sequencing a polypeptide by sequencing a plurality of fragments of the polypeptide. In some embodiments, sequencing a polypeptide comprises combining sequence information for a plurality of polypeptide fragments to identify and/or determine a sequence for the polypeptide. In some embodiments, combining sequence information may be performed by computer hardware and software. See “Devices for Sample Preparation and Sample Sequencing.” The methods described herein may allow for a set of related polypeptides, such as an entire proteome of an organism, to be sequenced. In some embodiments, a plurality of single molecule sequencing reactions are performed in parallel (e.g., on a single chip) according to aspects of the present application. For example, in some embodiments, a plurality of single molecule sequencing reactions are each performed in separate sample wells on a single chip or array.

In some embodiments, methods provided herein may be used for the sequencing and identification of an individual polypeptide in a sample comprising a complex mixture or an enriched mixture of polypeptides. In some embodiments, the application provides methods of uniquely identifying an individual polypeptide in a complex mixture or an enriched mixture of polypeptides. In some embodiments, an individual polypeptide is detected in a mixed sample by determining a partial amino acid sequence of the polypeptide. In some embodiments, the partial amino acid sequence of the polypeptide is within a contiguous stretch of approximately 5 to 50 amino acids.

Without wishing to be bound by any particular theory, it is believed that most human proteins can be identified using incomplete sequence information with reference to proteomic databases. For example, simple modeling of the human proteome has shown that approximately 98% of proteins can be uniquely identified by detecting just four types of amino acids within a stretch of 6 to 40 amino acids (see, e.g., Swaminathan, et al. PLoS Comput Biol. 2015, 11(2):e1004080; and Yao, et al. Phys. Biol. 2015, 12(5):055003). Therefore, a complex mixture or enriched mixture of polypeptides can be degraded (e.g., chemically degraded, enzymatically degraded) into short polypeptide fragments of approximately 6 to 40 amino acids, and sequencing of this polypeptide library would reveal the identity and abundance of each of the polypeptides present in the original complex mixture or enriched mixture. Compositions and methods for selective amino acid labeling and identifying polypeptides by determining partial sequence information are described in in detail in U.S. patent application Ser. No. 15/510,962, filed Sep. 15, 2015, titled “SINGLE MOLECULE PEPTIDE SEQUENCING,” which is incorporated by reference in its entirety.

Embodiments are capable of sequencing single polypeptide molecules with high accuracy, such as an accuracy of at least about 50%, 60%, 70%, 75%, 80%, 85%, 90%, 95%, 96%, 97%, 98%, 99%, 99.9%, 99.99%, 99.999%, or 99.9999%. In some embodiments, the target molecule used in single molecule sequencing is a polypeptide that is immobilized on a surface of a solid support such as a bottom surface or a sidewall surface of a sample well. The sample well also can contain other reagents needed for a sequencing reaction in accordance with the application, such as one or more suitable buffers, co-factors, labeled affinity reagents, and enzymes (e.g., catalytically active or inactive exopeptidase enzymes, which may be luminescently labeled or unlabeled).

Sequencing in accordance with the application, in some aspects, may involve immobilizing a polypeptide on a surface of a substrate (e.g., of a solid support, for example a chip, for example an integrated device as described herein). In some embodiments, a polypeptide may be immobilized on a surface of a sample well (e.g., on a bottom surface of a sample well) on a substrate. In some embodiments, the N-terminal amino acid of the polypeptide is immobilized (e.g., attached to the surface). In some embodiments, the C-terminal amino acid of the polypeptide is immobilized (e.g., attached to the surface). In some embodiments, one or more non-terminal amino acids are immobilized (e.g., attached to the surface). The immobilized amino acid(s) can be attached using any suitable covalent or non-covalent linkage, for example as described in this application. In some embodiments, a plurality of polypeptides are attached to a plurality of sample wells (e.g., with one polypeptide attached to a surface, for example a bottom surface, of each sample well), for example in an array of sample wells on a substrate.

Sequencing in accordance with the application, in some aspects, may be performed using a system that permits single molecule analysis. The system may include a sequencing device and an instrument configured to interface with the sequencing device. See “Devices for Sample Preparation and Sample Sequencing”.

### A. Labeled Affinity Reagents and Methods of Use

In some embodiments, methods provided herein comprise contacting a polypeptide with a labeled affinity reagent (also referred to herein as an amino acid recognition molecule, which may or may not comprise a label) that selectively binds one type of terminal amino acid. As used herein, in some embodiments, a terminal amino acid may refer to an amino-terminal amino acid of a polypeptide or a carboxy-terminal amino acid of a polypeptide. In some embodiments, a labeled affinity reagent selectively binds one type of terminal amino acid over other types of terminal amino acids. In some embodiments, a labeled affinity reagent selectively binds one type of terminal amino acid over an internal amino acid of the same type. In yet other embodiments, a labeled affinity reagent selectively binds one type of amino acid at any position of a polypeptide, e.g., the same type of amino acid as a terminal amino acid and an internal amino acid.

As used herein, in some embodiments, a type of amino acid refers to one of the twenty naturally occurring amino acids or a subset of types thereof. In some embodiments, a type of amino acid refers to a modified variant of one of the twenty naturally occurring amino acids or a subset of unmodified and/or modified variants thereof. Examples of modified amino acid variants include, without limitation, post-translationally-modified variants (e.g., acetylation, ADP-ribosylation, caspase cleavage, citrullination, formylation, N-linked glycosylation, 0-linked glycosylation, hydroxylation, methylation, myristoylation, neddylation, nitration, oxidation, palmitoylation, phosphorylation, prenylation, S-nitrosylation, sulfation, sumoylation, and ubiquitination), chemically modified variants, unnatural amino acids, and proteinogenic amino acids such as selenocysteine and pyrrolysine. In some embodiments, a subset of types of amino acids includes more than one and fewer than twenty amino acids having one or more similar biochemical properties. For example, in some embodiments, a type of amino acid refers to one type selected from amino acids with charged side chains (e.g., positively and/or negatively charged side chains), amino acids with polar side chains (e.g., polar uncharged side chains), amino acids with nonpolar side chains (e.g., nonpolar aliphatic and/or aromatic side chains), and amino acids with hydrophobic side chains.

In some embodiments, methods provided herein comprise contacting a polypeptide with one or more labeled affinity reagents that selectively bind one or more types of terminal amino acids. As an illustrative and non-limiting example, where four labeled affinity reagents are used in a method of the application, any one reagent selectively binds one type of terminal amino acid that is different from another type of amino acid to which any of the other three selectively binds (e.g., a first reagent binds a first type, a second reagent binds a second type, a third reagent binds a third type, and a fourth reagent binds a fourth type of terminal amino acid). For the purposes of this discussion, one or more labeled affinity reagents in the context of a method described herein may be alternatively referred to as a set of labeled affinity reagents.

In some embodiments, a set of labeled affinity reagents comprises at least one and up to six labeled affinity reagents. For example, in some embodiments, a set of labeled affinity reagents comprises one, two, three, four, five, or six labeled affinity reagents. In some embodiments, a set of labeled affinity reagents comprises ten or fewer labeled affinity reagents. In some embodiments, a set of labeled affinity reagents comprises eight or fewer labeled affinity reagents. In some embodiments, a set of labeled affinity reagents comprises six or fewer labeled affinity reagents. In some embodiments, a set of labeled affinity reagents comprises four or fewer labeled affinity reagents. In some embodiments, a set of labeled affinity reagents comprises three or fewer labeled affinity reagents. In some embodiments, a set of labeled affinity reagents comprises two or fewer labeled affinity reagents. In some embodiments, a set of labeled affinity reagents comprises four labeled affinity reagents. In some embodiments, a set of labeled affinity reagents comprises at least two and up to twenty (e.g., at least two and up to ten, at least two and up to eight, at least four and up to twenty, at least four and up to ten) labeled affinity reagents. In some embodiments, a set of labeled affinity reagents comprises more than twenty (e.g., 20 to 25, 20 to 30) affinity reagents. It should be appreciated, however, that any number of affinity reagents may be used in accordance with a method of the application to accommodate a desired use.

In accordance with the application, in some embodiments, one or more types of amino acids are identified by detecting luminescence of a labeled affinity reagent (e.g., an amino acid recognition molecule comprising a luminescent label). In some embodiments, a labeled affinity reagent comprises an affinity reagent that selectively binds one type of amino acid and a luminescent label having a luminescence that is associated with the affinity reagent. In this way, the luminescence (e.g., luminescence lifetime, luminescence intensity, and other luminescence properties described elsewhere herein) may be associated with the selective binding of the affinity reagent to identify an amino acid of a polypeptide. In some embodiments, a plurality of types of labeled affinity reagents may be used in a method according to the application, wherein each type comprises a luminescent label having a luminescence that is uniquely identifiable from among the plurality. Suitable luminescent labels may include luminescent molecules, such as fluorophore dyes, and are described elsewhere herein.

In some embodiments, one or more types of amino acids are identified by detecting one or more electrical characteristics of a labeled affinity reagent. In some embodiments, a labeled affinity reagent comprises an affinity reagent that selectively binds one type of amino acid and a conductivity label that is associated with the affinity reagent. In this way, the one or more electrical characteristics (e.g., charge, current oscillation color, and other electrical characteristics) may be associated with the selective binding of the affinity reagent to identify an amino acid of a polypeptide. In some embodiments, a plurality of types of labeled affinity reagents may be used in a method according to the application, wherein each type comprises a conductivity label that produces a change in an electrical signal (e.g., a change in conductance, such as a change in amplitude of conductivity and conductivity transitions of a characteristic pattern) that is uniquely identifiable from among the plurality. In some embodiments, the plurality of types of labeled affinity reagents each comprises a conductivity label having a different number of charged groups (e.g., a different number of negatively and/or positively charged groups). Accordingly, in some embodiments, a conductivity label is a charge label. Examples of charge labels include dendrimers, nanoparticles, nucleic acids and other polymers having multiple charged groups. In some embodiments, a conductivity label is uniquely identifiable by its net charge (e.g., a net positive charge or a net negative charge), by its charge density, and/or by its number of charged groups.

In some embodiments, an affinity reagent (e.g., an amino acid recognition molecule) may be engineered by one skilled in the art using conventionally known techniques. In some embodiments, desirable properties may include an ability to bind selectively and with high affinity to one type of amino acid only when it is located at a terminus (e.g., an N-terminus or a C-terminus) of a polypeptide. In yet other embodiments, desirable properties may include an ability to bind selectively and with high affinity to one type of amino acid when it is located at a terminus (e.g., an N-terminus or a C-terminus) of a polypeptide and when it is located at an internal position of the polypeptide.

As used herein, in some embodiments, the terms “selective” and “specific” (and variations thereof, e.g., selectively, specifically, selectivity, specificity) refer to a preferential binding interaction. For example, in some embodiments, a labeled affinity reagent that selectively binds one type of amino acid preferentially binds the one type over another type of amino acid. A selective binding interaction will discriminate between one type of amino acid (e.g., one type of terminal amino acid) and other types of amino acids (e.g., other types of terminal amino acids), typically more than about 10- to 100-fold or more (e.g., more than about 1,000- or 10,000-fold). Accordingly, it should be appreciated that a selective binding interaction can refer to any binding interaction that is uniquely identifiable to one type of amino acid over other types of amino acids. For example, in some aspects, the application provides methods of polypeptide sequencing by obtaining data indicative of association of one or more amino acid recognition molecules with a polypeptide molecule. In some embodiments, the data comprises a series of signal pulses corresponding to a series of reversible amino acid recognition molecule binding interactions with an amino acid of the polypeptide molecule, and the data may be used to determine the identity of the amino acid. As such, in some embodiments, a “selective” or “specific” binding interaction refers to a detected binding interaction that discriminates between one type of amino acid and other types of amino acids.

In some embodiments, a labeled affinity reagent (e.g., an amino acid recognition molecule) selectively binds one type of amino acid with a dissociation constant (K_{D}) of less than about 10⁻⁶ M (e.g., less than about 10⁻⁷ M, less than about 10⁻⁸ M, less than about 10⁻⁹ M, less than about 10⁻¹⁰ M, less than about 10⁻¹¹ M, less than about 10⁻¹² M, to as low as 10⁻¹⁶ M) without significantly binding to other types of amino acids. In some embodiments, a labeled affinity reagent selectively binds one type of amino acid (e.g., one type of terminal amino acid) with a K_{D} of less than about 100 nM, less than about 50 nM, less than about 25 nM, less than about 10 nM, or less than about 1 nM. In some embodiments, a labeled affinity reagent selectively binds one type of amino acid with a K_{D} between about 50 nM and about 50 μM (e.g., between about 50 nM and about 500 nM, between about 50 nM and about 5 μM, between about 500 nM and about 50 μM, between about 5 μM and about 50 μM, or between about 10 μM and about 50 μM). In some embodiments, an amino acid recognition molecule binds one type of amino acid with a KD of about 50 nM.

In some embodiments, a labeled affinity reagent (e.g., an amino acid recognition molecule) binds two or more types of amino acids with a KD of less than about 10⁻⁶ M (e.g., less than about 10⁻⁷ M, less than about 10⁻⁸ M, less than about 10⁻⁹ M, less than about 10⁻¹⁰ M, less than about 10⁻¹¹ M, less than about 10⁻¹² M, to as low as 10⁻¹⁶ M). In some embodiments, an amino acid recognition molecule binds two or more types of amino acids with a KD of less than about 100 nM, less than about 50 nM, less than about 25 nM, less than about 10 nM, or less than about 1 nM. In some embodiments, an amino acid recognition molecule binds two or more types of amino acids with a KD of between about 50 nM and about 50 μM (e.g., between about 50 nM and about 500 nM, between about 50 nM and about 5 μM, between about 500 nM and about 50 μM, between about 5 μM and about 50 μM, or between about 10 μM and about 50 μM). In some embodiments, an amino acid recognition molecule binds two or more types of amino acids with a KD of about 50 nM.

In some embodiments, a labeled affinity reagent (e.g., an amino acid recognition molecule) binds at least one type of amino acid with a dissociation rate (koff) of at least 0.1 s⁻¹. In some embodiments, the dissociation rate is between about 0.1 s⁻¹ and about 1,000 s⁻¹ (e.g., between about 0.5 s⁻¹ and about 500 s⁻¹, between about 0.1 s⁻¹ and about 100 s⁻¹, between about 1 s⁻¹ and about 100 s⁻¹, or between about 0.5 s⁻¹ and about 50 s⁻¹). In some embodiments, the dissociation rate is between about 0.5 s⁻¹ and about 20 s⁻¹. In some embodiments, the dissociation rate is between about 2 s⁻¹ and about 20 s⁻¹. In some embodiments, the dissociation rate is between about 0.5 s-1 and about 2 s⁻¹.

In some embodiments, the value for KD or koff can be a known literature value, or the value can be determined empirically. For example, the value for KD or koff can be measured in a single-molecule assay or an ensemble assay. In some embodiments, the value for koff can be determined empirically based on signal pulse information obtained in a single-molecule assay as described elsewhere herein. For example, the value for koff can be approximated by the reciprocal of the mean pulse duration. In some embodiments, an amino acid recognition molecule binds two or more types of amino acids with a different KD or koff for each of the two or more types. In some embodiments, a first KD or koff for a first type of amino acid differs from a second KD or koff for a second type of amino acid by at least 10% (e.g., at least 25%, at least 50%, at least 100%, or more). In some embodiments, the first and second values for KD or koff differ by about 10-25%, 25-50%, 50-75%, 75-100%, or more than 100%, for example by about 2-fold, 3-fold, 4-fold, 5-fold, or more.

In some embodiments, a labeled affinity reagent comprises a luminescent label (e.g., a label) and an affinity reagent that selectively binds one or more types of terminal amino acids of a polypeptide. In some embodiments, an affinity reagent is selective for one type of amino acid or a subset (e.g., fewer than the twenty common types of amino acids) of types of amino acids at a terminal position or at both terminal and internal positions.

As described herein, an affinity reagent (also known as a “recognition molecule”) may be any biomolecule capable of selectively or specifically binding one molecule over another molecule (e.g., one type of amino acid over another type of amino acid, as with an “amino acid recognition molecule” referred to herein). Affinity reagents (e.g., recognition molecules) include, for example, proteins and nucleic acids, which may be synthetic or recombinant. In some embodiments, an affinity reagent or recognition molecule may be an antibody or an antigen-binding portion of an antibody, or an enzymatic biomolecule, such as a peptidase, an aminotransferase, a ribozyme, an aptazyme, or a tRNA synthetase, including aminoacyl-tRNA synthetases and related molecules described in U.S. patent application Ser. No. 15/255,433, filed Sep. 2, 2016, titled “MOLECULES AND METHODS FOR ITERATIVE POLYPEPTIDE ANALYSIS AND PROCESSING”.

In some embodiments, an affinity reagent or recognition molecule of the application is a degradation pathway protein. Examples of degradation pathway proteins suitable for use as recognition molecules include, without limitation, N-end rule pathway proteins, such as Arg/N-end rule pathway proteins, Ac/N-end rule pathway proteins, and Pro/N-end rule pathway proteins. In some embodiments, a recognition molecule is an N-end rule pathway protein selected from a Gid4 protein, a Ubr1 UBR box protein, and a ClpS protein (e.g., ClpS2).

A peptidase, also referred to as a protease or proteinase, is an enzyme that catalyzes the hydrolysis of a peptide bond. Peptidases digest polypeptides into shorter fragments and may be generally classified into endopeptidases and exopeptidases, which cleave a polypeptide chain internally and terminally, respectively. In some embodiments, labeled affinity reagent comprises a peptidase that has been modified to inactivate exopeptidase or endopeptidase activity. In this way, labeled affinity reagent selectively binds without also cleaving the amino acid from a polypeptide. In yet other embodiments, a peptidase that has not been modified to inactivate exopeptidase or endopeptidase activity may be used. For example, in some embodiments, a labeled affinity reagent comprises a labeled exopeptidase.

In accordance with certain embodiments of the application, polypeptide sequencing methods may comprise iterative detection and cleavage at a terminal end of a polypeptide. In some embodiments, labeled exopeptidase may be used as a single reagent that performs both steps of detection and cleavage of an amino acid. As generically depicted, in some embodiments, labeled exopeptidase has aminopeptidase or carboxypeptidase activity such that it selectively binds and cleaves an N-terminal or C-terminal amino acid, respectively, from a polypeptide. It should be appreciated that, in certain embodiments, labeled exopeptidase may be catalytically inactivated by one skilled in the art such that labeled exopeptidase retains selective binding properties for use as a non-cleaving labeled affinity reagent, as described herein.

An exopeptidase generally requires a polypeptide substrate to comprise at least one of a free amino group at its amino-terminus or a free carboxyl group at its carboxy-terminus. In some embodiments, an exopeptidase in accordance with the application hydrolyses a bond at or near a terminus of a polypeptide. In some embodiments, an exopeptidase hydrolyses a bond not more than three residues from a polypeptide terminus. For example, in some embodiments, a single hydrolysis reaction catalyzed by an exopeptidase cleaves a single amino acid, a dipeptide, or a tripeptide from a polypeptide terminal end.

In some embodiments, an exopeptidase in accordance with the application is an aminopeptidase or a carboxypeptidase, which cleaves a single amino acid from an amino- or a carboxy-terminus, respectively. In some embodiments, an exopeptidase in accordance with the application is a dipeptidyl-peptidase or a peptidyl-dipeptidase, which cleave a dipeptide from an amino- or a carboxy-terminus, respectively. In yet other embodiments, an exopeptidase in accordance with the application is a tripeptidyl-peptidase, which cleaves a tripeptide from an amino-terminus. Peptidase classification and activities of each class or subclass thereof is well known and described in the literature (see, e.g., Gurupriya, V. S. & Roy, S. C. Proteases and Protease Inhibitors in Male Reproduction. Proteases in Physiology and Pathology 195-216 (2017); and Brix, K. & Stocker, W. Proteases: Structure and Function. Chapter 1).

An exopeptidase in accordance with the application may be selected or engineered based on the directionality of a sequencing reaction. For example, in embodiments of sequencing from an amino-terminus to a carboxy-terminus of a polypeptide, an exopeptidase comprises aminopeptidase activity. Conversely, in embodiments of sequencing from a carboxy-terminus to an amino-terminus of a polypeptide, an exopeptidase comprises carboxypeptidase activity. Examples of carboxypeptidases that recognize specific carboxy-terminal amino acids, which may be used as labeled exopeptidases or inactivated to be used as non-cleaving labeled affinity reagents described herein, have been described in the literature (see, e.g., Garcia-Guerrero, M. C., et al. (2018) PNAS 115(17)).

Suitable peptidases for use as cleaving reagents and/or affinity reagents (e.g., recognition molecules) include aminopeptidases that selectively bind one or more types of amino acids. In some embodiments, an aminopeptidase recognition molecule is modified to inactivate aminopeptidase activity. In some embodiments, an aminopeptidase cleaving reagent is non-specific such that it cleaves most or all types of amino acids from a terminal end of a polypeptide. In some embodiments, an aminopeptidase cleaving reagent is more efficient at cleaving one or more types of amino acids from a terminal end of a polypeptide as compared to other types of amino acids at the terminal end of the polypeptide. For example, an aminopeptidase in accordance with the application specifically cleaves alanine, arginine, asparagine, aspartic acid, cysteine, glutamine, glutamic acid, glycine, histidine, isoleucine, leucine, lysine, methionine, phenylalanine, proline, selenocysteine, serine, threonine, tryptophan, tyrosine, and/or valine. In some embodiments, an aminopeptidase is a proline aminopeptidase. In some embodiments, an aminopeptidase is a proline iminopeptidase. In some embodiments, an aminopeptidase is a glutamate/aspartate-specific aminopeptidase. In some embodiments, an aminopeptidase is a methionine-specific aminopeptidase. In some embodiments, an aminopeptidase is an aminopeptidase set forth in TABLE 1. In some embodiments, an aminopeptidase cleaving reagent cleaves a peptide substrate set forth in TABLE 1.

In some embodiments, an aminopeptidase is a non-specific aminopeptidase. In some embodiments, a non-specific aminopeptidase is a zinc metalloprotease. In some embodiments, a non-specific aminopeptidase is an aminopeptidase set forth in TABLE 2. In some embodiments, a non-specific aminopeptidase cleaves a peptide substrate set forth in TABLE 2.

Accordingly, in some embodiments, the application provides an aminopeptidase (e.g., an aminopeptidase recognition molecule, an aminopeptidase cleaving reagent) having an amino acid sequence selected from TABLE 1 or TABLE 2 (or having an amino acid sequence that has at least 50%, at least 60%, at least 70%, at least 80%, 80-90%, 90-95%, 95-99%, or higher, amino acid sequence identity to an amino acid sequence selected from TABLE 1 or TABLE 2). In some embodiments, an aminopeptidase has 25-50%, 50-60%, 60-70%, 70-80%, 80-90%, 90-95%, or 95-99%, or higher, amino acid sequence identity to an aminopeptidase listed in TABLE 1 or TABLE 2. In some embodiments, an aminopeptidase is a modified aminopeptidase and includes one or more amino acid mutations relative to a sequence set forth in TABLE 1 or TABLE 2.

TABLE 1
Non-limiting examples of aminopeptidases.

| | SEQ | |
|---|---|---|
| | ID | |
| Name | NO: | Sequence |
| L. pneumophila M1 | 1 | MGSSHHHHHHSSGLVPRGSHMMVKQGVFMKTDQSKVKKLSDYKSLDYF |
| Aminopeptidase | | VIHVDLQIDLSKKPVESKARLTVVPNLNVDSHSNDLVLDGENMTLVSLQ |
| (Glu/Asp Specific) | | MNDNLLKENEYELTKDSLIIKNIPQNTPFTIEMTSLLGENTDLFGLYETEGV |
| | | ALVKAESEGLRRVFYLPDRPDNLATYKTTIIANQEDYPVLLSNGVLIEKKE |
| | | LPLGLHSVTWLDDVPKPSYLFALVAGNLQRSVTYYQTKSGRELPIEFYVP |
| | | PSATSKCDFAKEVLKEAMAWDERTFNLECALRQHMVAGVDKYASGASE |
| | | PTGLNLFNTENLFASPETKTDLGILRVLEVVAHEFFHYWSGDRVTIRDWF |
| | | NLPLKEGLTTFRAAMFREELFGTDLIRLLDGKNLDERAPRQSAYTAVRSL |
| | | YTAAAYEKSADIFRMMMLFIGKEPFIEAVAKFFKDNDGGAVTLEDFIESIS |
| | | NSSGKDLRSFLSWFTESGIPELIVTDELNPDTKQYFLKIKTVNGRNRPIPIL |
| | | MGLLDSSGAEIVADKLLIVDQEEIEFQFENIQTRPIPSLLRSFSAPVHMKYE |
| | | YSYQDLLLLMQFDTNLYNRCEAAKQLISALINDFCIGKKIELSPQFFAVYK |
| | | ALLSDNSLNEWMLAELITLPSLEELIENQDKPDFEKLNEGRQLIQNALANE |
| | | LKTDFYNLLFRIQISGDDDKQKLKGFDLKQAGLRRLKSVCFSYLLNVDFE |
| | | KTKEKLILQFEDALGKNMTETALALSMLCEINCEEADVALEDYYHYWKN |
| | | DPGAVNNWFSIQALAHSPDVIERVKKLMRHGDFDLSNPNKVYALLGSFIK |
| | | NPFGFHSVTGEGYQLVADAIFDLDKINPTLAANLTEKFTYWDKYDVNRQ |
| | | AMMISTLKIIYSNATSSDVRTMAKKGLDKVKEDLPLPIHLTFHGGSTMQD |
| | | RTAQLIADGNKENAYQLH |
| E. coli methionine | 2 | MAHHHHHHMGTAISIKTPEDIEKMRVAGRLAAEVLEMIEPYVKPGVSTGE |
| aminopeptidase | | LDRICNDYIVNEQHAVSACLGYHGYPKSVCISINEVVCHGIPDDAKLLKD |
| (Met specific) | | GDIVNIDVTVIKDGFHGDTSKMFIVGKPTIMGERLCRITQESLYLALRMVK |
| | | PGINLREIGAAIQKFVEAEGFSVVREYCGHGIGRGFHEEPQVLHYDSRETN |
| | | VVLKPGMTFTIEPMVNAGKKEIRTMKDGWTVKTKDRSLSAQYEHTIVVT |
| | | DNGCEILTLRKDDTIPAIISHD |
| M. smegmatis | 3 | MAHHHHHHMGTLEANTNGPGSMLSRMPVSSRTVPFGDHETWVQVTTPE |
| Proline | | NAQPHALPLIVLHGGPGMAHNYVANIAALADETGRTVIHYDQVGCGNST |
| iminopeptidase | | HLPDAPADFWTPQLFVDEFHAVCTALGIERYHVLGQSWGGMLGAEIAVR |
| (Pro specific) | | QPSGLVSLAICNSPASMRLWSEAAGDLRAQLPAETRAALDRHEAAGTITH |
| | | PDYLQAAAEFYRRHVCRVVPTPQDFADSVAQMEAEPTVYHTMNGPNEF |
| | | HVVGTLGDWSVIDRLPDVTAPVLVIAGEHDEATPKTWQPFVDHIPDVRSH |
| | | VFPGTSHCTHLEKPEEFRAVVAQFLHQHDLAADARV |
| Y. pestis Proline | 4 | MTQQEYQNRRQALLAKMAPGSAAIIFAAPEATRSADSEYPYRQNSDFSYL |
| iminopeptidase | | TGFNEPEAVLILVKSDETHNHSVLFNRIRDLTAEIWFGRRLGQEAAPTKLA |
| (Pro Specific) | | VDRALPFDEINEQLYLLLNRLDVIYHAQGQYAYADNIVFAALEKLRHGFR |
| | | KNLRAPATLTDWRPWLHEMRLFKSAEEIAVLRRAGEISALAHTRAMEKC |
| | | RPGMFEYQLEGEILHEFTRHGARYPAYNTIVGGGENGCILHYTENECELR |
| | | DGDLVLIDAGCEYRGYAGDITRTFPVNGKFTPAQRAVYDIVLAAINKSLT |
| | | LFRPGTSIREVTEEVVRIMVVGLVELGILKGDIEQLIAEQAHRPFFMHGLSH |
| | | WLGMDVHDVGDYGSSDRGRILEPGMVLTVEPGLYIAPDADVPPQYRGIGI |
| | | RIEDDIVITATGNENLTASVVKDPDDIEALMALNHAGENLYFQEHHHHHH |
| P. furiosus | 5 | MDTEKLMKAGEIAKKVREKAIKLARPGMLLLELAESIEKMIMELGGKPAF |
| Methionine | | PVNLSINEIAAHYTPYKGDTTVLKEGDYLKIDVGVHIDGFIADTAVTVRVG |
| aminopeptidase | | MEEDELMEAAKEALNAAISVARAGVEIKELGKAIENEIRKRGFKPIVNLSG |
| | | HKIERYKLHAGISIPNIYRPHDNYVLKEGDVFAIEPFATIGAGQVIEVPPTLI |
| | | YMYVRDVPVRVAQARFLLAKIKREYGTLPFAYRWLQNDMPEGQLKLAL |
| | | KTLEKAGAIYGYPVLKEIRNGIVAQFEHTIIVEKDSVIVTQDMINKSTLE |
| Aeromonas sobria | 6 | HMSSPLHYVLDGIHCEPHFFTVPLDHQQPDDEETITLFGRTLCRKDRLDDE |
| Proline | | LPWLLYLQGGPGFGAPRPSANGGWIKRALQEFRVLLLDQRGTGHSTPIHA |
| aminopeptidase | | ELLAHLNPRQQADYLSHFRADSIVRDAELIREQLSPDHPWSLLGQSFGGFC |
| | | SLTYLSLFPDSLHEVYLTGGVAPIGRSADEVYRATYQRVADKNRAFFARF |
| | | PHAQAIANRLATHLQRHDVRLPNGQRLTVEQLQQQGLDLGASGAFEELY |
| | | YLLEDAFIGEKLNPAFLYQVQAMQPFNTNPVFAILHELIYCEGAASHWAA |
| | | ERVRGEFPALAWAQGKDFAFTGEMIFPWMFEQFRELIPLKEAAHLLAEKA |
| | | DWGPLYDPVQLARNKVPVACAVYAEDMYVEFDYSRETLKGLSNSRAWI |
| | | TNEYEHNGLRVDGEQILDRLIRLNRDCLE |
| Pyrococcus furiosus | 7 | MKERLEKLVKFMDENSIDRVFIAKPVNVYYFSGTSPLGGGYIIVDGDEATL |
| Proline | | YVPELEYEMAKEESKLPVVKFKKFDEIYEILKNTETLGIEGTLSYSMVENF |
| Aminopeptidase (X- | | KEKSNVKEFKKIDDVIKDLRIIKTKEEIEIIEKACEIADKAVMAAIEEITEGK |
| /-Pro) | | REREVAAKVEYLMKMNGAEKPAFDTIIASGHRSALPHGVASDKRIERGDL |
| | | VVIDLGALYNHYNSDITRTIVVGSPNEKQREIYEIVLEAQKRAVEAAKPG |
| | | MTAKELDSIAREIIKEYGYGDYFIHSLGHGVGLEIHEWPRISQYDETVLKE |
| | | GMVITIEPGIYIPKLGGVRIEDTVLITENGAKRLTKTERELL |
| Elizabethkingia | 8 | MIPITTPVGNFKVWTKRFGTNPKIKVLLLHGGPAMTHEYMECFETFFQRE |
| meningoseptica | | GFEFYEYDQLGSYYSDQPTDEKLWNIDRFVDEVEQVRKAIHADKENFYV |
| Proline | | LGNSWGGILAMEYALKYQQNLKGLIVANMMASAPEYVKYAEVLSKQM |
| aminopeptidase | | KPEVLAEVRAIEAKKDYANPRYTELLFPNYYAQHICRLKEWPDALNRSLK |
| | | HVNSTVYTLMQGPSELGMSSDARLAKWDIKNRLHEIATPTLMIGARYDT |
| | | MDPKAMEEQSKLVQKGRYLYCPNGSHLAMWDDQKVFMDGVIKFIKDV |
| | | DTKSFN |
| Aeromonas sobria | 9 | HMSSPLHYVLDGIHCEPHFFTVPLDHQQPDDEETITLFGRTLCRKDRLDDE |
| Proline | | LPWLLYLQGGPGFGAPRPSANGGWIKRALQEFRVLLLDQRGTGHSTPIHA |
| aminopeptidase | | ELLAHLNPRQQADYLSHFRADSIVRDAELIREQLSPDHPWSLLGQSFGGFC |
| | | SLTYLSLFPDSLHEVYLTGGVAPIGRSADEVYRATYQRVADKNRAFFARF |
| | | PHAQAIANRLATHLQRHDVRLPNGQRLTVEQLQQQGLDLGASGAFEELY |
| | | YLLEDAFIGEKLNPAFLYQVQAMQPFNTNPVFAILHELIYCEGAASHWAA |
| | | ERVRGEFPALAWAQGKDFAFTGEMIFPWMFEQFRELIPLKEAAHLLAEKA |
| | | DWGPLYDPVQLARNKVPVACAVYAEDMYVEFDYSRETLKGLSNSRAWI |
| | | TNEYEHNGLRVDGEQILDRLIRLNRDCLE |
| N. gonorrhoeae | 10 | MYEIKQPFHSGYLQVSEIHQIYWEESGNPDGVPVIFLHGGPGAGASPECRG |
| Proline | | FFNPDVFRIVIIDQRGCGRSHPYACAEDNTTWDLVADIEKVREMLGIGKW |
| Iminopeptidase | | LVFGGSWGSTLSLAYAQTHPERVKGLVLRGIFLCRPSETAWLNEAGGVSR |
| | | IYPEQWQKFVAPIAENRRNRLIEAYHGLLFHQDEEVCLSAAKAWADWES |
| | | YLIRFEPEGVDEDAYASLAIARLENHYFVNGGWLQGDKAILNNIGKIRHIP |
| | | TVIVQGRYDLCTPMQSAWELSKAFPEAELRVVQAGHCAFDPPLADALVQ |
| | | AVEDILPRLL |

TABLE 2
Non-limiting example of non-specific aminopeptidases

| | SEQ | |
|---|---|---|
| | ID | |
| Name | NO: | Sequence |
| E. coli | 11 | MGSSHHHHHHSSGENLYFQGHMTQQPQAKYRHDYRAPDYQITDIDLTFD |
| Aminopeptidase N | | LDAQKTVVTAVSQAVRHGASDAPLRLNGEDLKLVSVHINDEPWTAWKE |
| (Zinc | | EEGALVISNLPERFTLKIINEISPAANTALEGLYQSGDALCTQCEAEGFRHIT |
| Metalloprotease)* | | YYLDRPDVLARFTTKIIADKIKYPFLLSNGNRVAQGELENGRHWVQWQD |
| | | PFPKPCYLFALVAGDFDVLRDTFTTRSGREVALELYVDRGNLDRAPWAM |
| | | TSLKNSMKWDEERFGLEYDLDIYMIVAVDFFNMGAMENKGLNIFNSKYV |
| | | LARTDTATDKDYLDIERVIGHEYFHNWTGNRVTCRDWFQLSLKEGLTVF |
| | | RDQEFSSDLGSRAVNRINNVRTMRGLQFAEDASPMAHPIRPDMVIEMNNF |
| | | YTLTVYEKGAEVIRMIHTLLGEENFQKGMQLYFERHDGSAATCDDFVQA |
| | | MEDASNVDLSHFRRWYSQSGTPIVTVKDDYNPETEQYTLTISQRTPATPD |
| | | QAEKQPLHIPFAIELYDNEGKVIPLQKGGHPVNSVLNVTQAEQTFVFDNV |
| | | YFQPVPALLCEFSAPVKLEYKWSDQQLTFLMRHARNDFSRWDAAQSLLA |
| | | TYIKLNVARHQQGQPLSLPVHVADAFRAVLLDEKIDPALAAEILTLPSVNE |
| | | MAELFDIIDPIAIAEVREALTRTLATELADELLAIYNANYQSEYRVEHEDIA |
| | | KRTLRNACLRFLAFGETHLADVLVSKQFHEANNMTDALAALSAAVAAQL |
| | | PCRDALMQEYDDKWHQNGLVMDKWFILQATSPAANVLETVRGLLQHRS |
| | | FTMSNPNRIRSLIGAFAGSNPAAFHAEDGSGYLFLVEMLTDLNSRNPQVAS |
| | | RLIEPLIRLKRYDAKRQEKMRAALEQLKGLENLSGDLYEKITKALA |
| P. falciparum M1 | 12 | PKIHYRKDYKPSGFIINQVTLNINIHDQETIVRSVLDMDISKHNVGEDLVFD |
| aminopeptidase** | | GVGLKINEISINNKKLVEGEEYTYDNEFLTIFSKFVPKSKFAFSSEVIIHPET |
| | | NYALTGLYKSKNIIVSQCEATGFRRITFFIDRPDMMAKYDVTVTADKEKY |
| | | PVLLSNGDKVNEFEIPGGRHGARFNDPPLKPCYLFAVVAGDLKHLSATYI |
| | | TKYTKKKVELYVFSEEKYVSKLQWALECLKKSMAFDEDYFGLEYDLSRL |
| | | NLVAVSDFNVGAMENKGLNIFNANSLLASKKNSIDFSYARILTVVGHEYF |
| | | HQYTGNRVTLRDWFQLTLKEGLTVHRENLFSEEMTKTVTTRLSHVDLLR |
| | | SVQFLEDSSPLSHPIRPESYVSMENFYTTTVYDKGSEVMRMYLTILGEEYY |
| | | KKGFDIYIKKNDGNTATCEDFNYAMEQAYKMKKADNSANLNQYLLWFS |
| | | QSGTPHVSFKYNYDAEKKQYSIHVNQYTKPDENQKEKKPLFIPISVGLINP |
| | | ENGKEMISQTTLELTKESDTFVFNNIAVKPIPSLFRGFSAPVYIEDQLTDEE |
| | | RILLLKYDSDAFVRYNSCTNIYMKQILMNYNEFLKAKNEKLESFQLTPVN |
| | | AQFIDAIKYLLEDPHADAGFKSYIVSLPQDRYIINFVSNLDTDVLADTKEYI |
| | | YKQIGDKLNDVYYKMFKSLEAKADDLTYFNDESHVDFDQMNMRTLRNT |
| | | LLSLLSKAQYPNILNEIIEHSKSPYPSNWLTSLSVSAYFDKYFELYDKTYKL |
| | | SKDDELLLQEWLKTVSRSDRKDIYEILKKLENEVLKDSKNPNDIRAVYLPF |
| | | TNNLRRFHDISGKGYKLIAEVITKTDKFNPMVATQLCEPFKLWNKLDTKR |
| | | QELMLNEMNTMLQEPQISNNLKEYLLRLTNK |
| NPEPPS | 13 | MGSSHHHHHHSSGMWLAAAAPSLARRLLFLGPPPPPLLLLVFSRSSRRRL |
| | | HSLGLAAMPEKRPFERLPADVSPINYSLCLKPDLLDFTFEGKLEAAAQVR |
| | | QATNQIVMNCADIDIITASYAPEGDEEIHATGFNYQNEDEKVTLSFPSTLQ |
| | | TGTGTLKIDFVGELNDKMKGFYRSKYTTPSGEVRYAAVTQFEATDARRA |
| | | FPCWDEPAIKATFDISLVVPKDRVALSNMNVIDRKPYPDDENLVEVKFAR |
| | | TPVMSTYLVAFVVGEYDFVETRSKDGVCVRVYTPVGKAEQGKFALEVA |
| | | AKTLPFYKDYFNVPYPLPKIDLIAIADFAAGAMENWGLVTYRETALLIDPK |
| | | NSCSSSRQWVALVVGHELAHQWFGNLVTMEWWTHLWLNEGFASWIEY |
| | | LCVDHCFPEYDIWTQFVSADYTRAQELDALDNSHPIEVSVGHPSEVDEIFD |
| | | AISYSKGASVIRMLHDYIGDKDFKKGMNMYLTKFQQKNAATEDLWESLE |
| | | NASGKPIAAVMNTWTKQMGFPLIYVEAEQVEDDRLLRLSQKKFCAGGSY |
| | | VGEDCPQWMVPITISTSEDPNQAKLKILMDKPEMNVVLKNVKPDQWVKL |
| | | NLGTVGFYRTQYSSAMLESLLPGIRDLSLPPVDRLGLQNDLFSLARAGIIST |
| | | VEVLKVMEAFVNEPNYTVWSDLSCNLGILSTLLSHTDFYEEIQEFVKDVFS |
| | | PIGERLGWDPKPGEGHLDALLRGLVLGKLGKAGHKATLEEARRRFKDHV |
| | | EGKQILSADLRSPVYLTVLKHGDGTTLDIMLKLHKQADMQEEKNRIERVL |
| | | GATLLPDLIQKVLTFALSEEVRPQDTVSVIGGVAGGSKHGRKAAWKFIKD |
| | | NWEELYNRYQGGFLISRLIKLSVEGFAVDKMAGEVKAFFESHPAPSAERTI |
| | | QQCCENILLNAAWLKRDAESIHQYLLQRKASPPTV |
| NPEPPS E366V | 14 | MGSSHHHHHHSSGMWLAAAAPSLARRLLFLGPPPPPLLLLVFSRSSRRRL |
| | | HSLGLAAMPEKRPFERLPADVSPINYSLCLKPDLLDFTFEGKLEAAAQVR |
| | | QATNQIVMNCADIDIITASYAPEGDEEIHATGFNYQNEDEKVTLSFPSTLQ |
| | | TGTGTLKIDFVGELNDKMKGFYRSKYTTPSGEVRYAAVTQFEATDARRA |
| | | FPCWDEPAIKATFDISLVVPKDRVALSNMNVIDRKPYPDDENLVEVKFAR |
| | | TPVMSTYLVAFVVGEYDFVETRSKDGVCVRVYTPVGKAEQGKFALEVA |
| | | AKTLPFYKDYFNVPYPLPKIDLIAIADFAAGAMENWGLVTYRETALLIDPK |
| | | NSCSSSRQWVALVVGHVLAHQWFGNLVTMEWWTHLWLNEGFASWIEY |
| | | LCVDHCFPEYDIWTQFVSADYTRAQELDALDNSHPIEVSVGHPSEVDEIFD |
| | | AISYSKGASVIRMLHDYIGDKDFKKGMNMYLTKFQQKNAATEDLWESLE |
| | | NASGKPIAAVMNTWTKQMGFPLIYVEAEQVEDDRLLRLSQKKFCAGGSY |
| | | VGEDCPQWMVPITISTSEDPNQAKLKILMDKPEMNVVLKNVKPDQWVKL |
| | | NLGTVGFYRTQYSSAMLESLLPGIRDLSLPPVDRLGLQNDLFSLARAGIIST |
| | | VEVLKVMEAFVNEPNYTVWSDLSCNLGILSTLLSHTDFYEEIQEFVKDVFS |
| | | PIGERLGWDPKPGEGHLDALLRGLVLGKLGKAGHKATLEEARRRFKDHV |
| | | EGKQILSADLRSPVYLTVLKHGDGTTLDIMLKLHKQADMQEEKNRIERVL |
| | | GATLLPDLIQKVLTFALSEEVRPQDTVSVIGGVAGGSKHGRKAAWKFIKD |
| | | NWEELYNRYQGGFLISRLIKLSVEGFAVDKMAGEVKAFFESHPAPSAERTI |
| | | QQCCENILLNAAWLKRDAESIHQYLLQRKASPPTV |
| Francisella | 15 | MIYEFVMTDPKIKYLKDYKPSNYLIDETHLIFELDESKTRVTANLYIVANR |
| tularensis | | ENRENNTLVLDGVELKLLSIKLNNKHLSPAEFAVNENQLIINNVPEKFVLQ |
| Aminopeptidase N | | TVVEINPSANTSLEGLYKSGDVFSTQCEATGFRKITYYLDRPDVMAAFTV |
| | | KIIADKKKYPIILSNGDKIDSGDISDNQHFAVWKDPFKKPCYLFALVAGDL |
| | | ASIKDTYITKSQRKVSLEIYAFKQDIDKCHYAMQAVKDSMKWDEDRFGL |
| | | EYDLDTFMIVAVPDFNAGAMENKGLNIFNTKYIMASNKTATDKDFELVQ |
| | | SVVGHEYFHNWTGDRVTCRDWFQLSLKEGLTVFRDQEFTSDLNSRDVKR |
| | | IDDVRIIRSAQFAEDASPMSHPIRPESYIEMNNFYTVTVYNKGAEIIRMIHTL |
| | | LGEEGFQKGMKLYFERHDGQAVTCDDFVNAMADANNRDFSLFKRWYA |
| | | QSGTPNIKVSENYDASSQTYSLTLEQTTLPTADQKEKQALHIPVKMGLINP |
| | | EGKNIAEQVIELKEQKQTYTFENIAAKPVASLFRDFSAPVKVEHKRSEKDL |
| | | LHIVKYDNNAFNRWDSLQQIATNIILNNADLNDEFLNAFKSILHDKDLDK |
| | | ALISNALLIPIESTIAEAMRVIMVDDIVLSRKNVVNQLADKLKDDWLAVY |
| | | QQCNDNKPYSLSAEQIAKRKLKGVCLSYLMNASDQKVGTDLAQQLFDN |
| | | ADNMTDQQTAFTELLKSNDKQVRDNAINEFYNRWRHEDLVVNKWLLSQ |
| | | AQISHESALDIVKGLVNHPAYNPKNPNKVYSLIGGFGANFLQYHCKDGLG |
| | | YAFMADTVLALDKFNHQVAARMARNLMSWKRYDSDRQAMMKNALEKI |
| | | KASNPSKNVFEIVSKSLES |
| Pyrococcus | 16 | MGSSHHHHHHSSGMEVRNMVDYELLKKVVEAPGVSGYEFLGIRDVVIEE |
| horikoshii TET | | IKDYVDEVKVDKLGNVIAHKKGEGPKVMIAAHMDQIGLMVTHIEKNGFL |
| Aminopeptidase | | RVAPIGGVDPKTLIAQRFKVWIDKGKFIYGVGASVPPHIQKPEDRKKAPD |
| | | WDQIFIDIGAESKEEAEDMGVKIGTVITWDGRLERLGKHRFVSIAFDDRIA |
| | | VYTILEVAKQLKDAKADVYFVATVQEEVGLRGARTSAFGIEPDYGFAIDV |
| | | TIAADIPGTPEHKQVTHLGKGTAIKIMDRSVICHPTIVRWLEELAKKHEIPY |
| | | QLEILLGGGTDAGAIHLTKAGVPTGALSVPARYIHSNTEVVDERDVDATV |
| | | ELMTKALENIHELKI |
| T. aquaticus | 17 | MDAFTENLNKLAELAIRVGLNLEEGQEIVATAPIEAVDFVRLLAEKAYEN |
| Aminopeptidase T | | GASLFTVLYGDNLIARKRLALVPEAHLDRAPAWLYEGMAKAFHEGAARL |
| | | AVSGNDPKALEGLPPERVGRAQQAQSRAYRPTLSAITEFVTNWTIVPFAH |
| | | PGWAKAVFPGLPEEEAVQRLWQAIFQATRVDQEDPVAAWEAHNRVLHA |
| | | KVAFLNEKRFHALHFQGPGTDLTVGLAEGHLWQGGATPTKKGRLCNPNL |
| | | PTEEVFTAPHRERVEGVVRASRPLALSGQLVEGLWARFEGGVAVEVGAE |
| | | KGEEVLKKLLDTDEGARRLGEVALVPADNPIAKTGLVFFDTLFDENAASH |
| | | IAFGQAYAENLEGRPSGEEFRRRGGNESMVHVDWMIGSEEVDVDGLLED |
| | | GTRVPLMRRGRWVI |
| Bacillus | 18 | MAKLDETLTMLKALTDAKGVPGNEREARDVMKTYIAPYADEVTTDGLG |
| stearothermophilus | | SLIAKKEGKSGGPKVMIAGHLDEVGFMVTQIDDKGFIRFQTLGGWWSQV |
| Peptidase M28 | | MLAQRVTIVTKKGDITGVIGSKPPHILPSEARKKPVEIKDMFIDIGATSREE |
| | | AMEWGVRPGDMIVPYFEFTVLNNEKMLLAKAWDNRIGCAVAIDVLKQL |
| | | KGVDHPNTVYGVGTVQEEVGLRGARTAAQFIQPDIAFAVDVGIAGDTPG |
| | | VSEKEAMGKLGAGPHIVLYDATMVSHRGLREFVIEVAEELNIPHHFDAMP |
| | | GVGTDAGAIHLTGIGVPSLTIAIPTRYIHSHAAILHRDDYENTVKLLVEVIK |
| | | RLDADKVKQLTFDE |
| Vibrio cholera | 19 | MEDKVWISMGADAVGSLNPALSESLLPHSFASGSQVWIGEVAIDELAELS |
| Aminopeptidase | | HTMHEQHNRCGGYMVHTSAQGAMAALMMPESIANFTIPAPSQQDLVNA |
| | | WLPQVSADQITNTIRALSSFNNRFYTTTSGAQASDWLANEWRSLISSLPGS |
| | | RIEQIKHSGYNQKSVVLTIQGSEKPDEWVIVGGHLDSTLGSHTNEQSIAPG |
| | | ADDDASGIASLSEIIRVLRDNNFRPKRSVALMAYAAEEVGLRGSQDLANQ |
| | | YKAQGKKVVSVLQLDMTNYRGSAEDIVFITDYTDSNLTQFLTTLIDEYLP |
| | | ELTYGYDRCGYACSDHASWHKAGFSAAMPFESKFKDYNPKIHTSQDTLA |
| | | NSDPTGNHAVKFTKLGLAYVIEMANAGSSQVPDDSVLQDGTAKINLSGA |
| | | RGTQKRFTFELSQSKPLTIQTYGGSGDVDLYVKYGSAPSKSNWDCRPYQN |
| | | GNRETCSFNNAQPGIYHVMLDGYTNYNDVALKASTQHHHHHH |
| Photobacterium | 20 | MEDKVWISIGSDASQTVKSVMQSNARSLLPESLASNGPVWVGQVDYSQL |
| halotolerans | | AELSHHMHEDHQRCGGYMVHSSPESAIAASNMPQSLVAFSIPEISQQDTV |
| Aminopeptidasej | | NAWLPQVNSQAITGTITSLTSFINRFYTTTSGAQASDWLANEWRSLSASLP |
| | | NASVRQVSHFGYNQKSVVLTITGSEKPDEWIVLGGHLDSTIGSHTNEQSV |
| | | APGADDDASGIASVTEIIRVLSENNFQPKRSIAFMAYAAEEVGLRGSQDLA |
| | | NQYKAEGKQVISALQLDMTNYKGSVEDIVFITDYTDSNLTTFLSQLVDEY |
| | | LPSLTYGFDTCGYACSDHASWHKAGFSAAMPFEAKFNDYNPMIHTPNDT |
| | | LQNSDPTASHAVKFTKLGLAYAIEMASTTGGTPPPTGNVLKDGVPVNGLS |
| | | GATGSQVHYSFELPAQKNLQISTAGGSGDVDLYVSFGSEATKQNWDCRP |
| | | YRNGNNEVCTFAGATPGTYSIMLDGYRQFSGVTLKASTQHHHHHH |
| Yersinia pestis | 21 | MTQQPQAKYRHDYRAPDYTITDIDLDFALDAQKTTVTAVSKVKRQGTDV |
| AminopeptidaseN | | TPLILNGEDLTLISVSVDGQAWPHYRQQDNTLVIEQLPADFTLTIVNDIHPA |
| | | TNSALEGLYLSGEALCTQCEAEGFRHITYYLDRPDVLARFTTRIVADKSRY |
| | | PYLLSNGNRVGQGELDDGRHWVKWEDPFPKPSYLFALVAGDFDVLQDK |
| | | FITRSGREVALEIFVDRGNLDRADWAMTSLKNSMKWDETRFGLEYDLDI |
| | | YMIVAVDFFNMGAMENKGLNVFNSKYVLAKAETATDKDYLNIEAVIGHE |
| | | YFHNWTGNRVTCRDWFQLSLKEGLTVFRDQEFSSDLGSRSVNRIENVRV |
| | | MRAAQFAEDASPMAHAIRPDKVIEMNNFYTLTVYEKGSEVIRMMHTLLG |
| | | EQQFQAGMRLYFERHDGSAATCDDFVQAMEDVSNVDLSLFRRWYSQSG |
| | | TPLLTVHDDYDVEKQQYHLFVSQKTLPTADQPEKLPLHIPLDIELYDSKGN |
| | | VIPLQHNGLPVHHVLNVTEAEQTFTFDNVAQKPIPSLLREFSAPVKLDYPY |
| | | SDQQLTFLMQHARNEFSRWDAAQSLLATYIKLNVAKYQQQQPLSLPAHV |
| | | ADAFRAILLDEHLDPALAAQILTLPSENEMAELFTTIDPQAISTVHEAITRC |
| | | LAQELSDELLAVYVANMTPVYRIEHGDIAKRALRNTCLNYLAFGDEEFAN |
| | | KLVSLQYHQADNMTDSLAALAAAVAAQLPCRDELLAAFDVRWNHDGL |
| | | VMDKWFALQATSPAANVLVQVRTLLKHPAFSLSNPNRTRSLIGSFASGNP |
| | | AAFHAADGSGYQFLVEILSDLNTRNPQVAARLIEPLIRLKRYDAGRQALM |
| | | RKALEQLKTLDNLSGDLYEKITKALAAHHHHHH |
| Vibrio anguillarum | 22 | MEEKVWISIGGDATQTALRSGAQSLLPENLINQTSVWVGQVPVSELATLS |
| Aminopeptidase | | HEMHENHQRCGGYMVHPSAQSAMSVSAMPLNLNAFSAPEITQQTTVNA |
| | | WLPSVSAQQITSTITTLTQFKNRFYTTSTGAQASNWIADHWRSLSASLPAS |
| | | KVEQITHSGYNQKSVMLTITGSEKPDEWVVIGGHLDSTLGSRTNESSIAPG |
| | | ADDDASGIAGVTEIIRLLSEQNFRPKRSIAFMAYAAEEVGLRGSQDLANRF |
| | | KAEGKKVMSVMQLDMTNYQGSREDIVFITDYTDSNFTQYLTQLLDEYLP |
| | | SLTYGFDTCGYACSDHASWHAVGYPAAMPFESKFNDYNPNIHSPQDTLQ |
| | | NSDPTGFHAVKFTKLGLAYVVEMGNASTPPTPSNQLKNGVPVNGLSASR |
| | | NSKTWYQFELQEAGNLSIVLSGGSGDADLYVKYQTDADLQQYDCRPYRS |
| | | GNNETCQFSNAQPGRYSILLHGYNNYSNASLVANAQHHHHHH |
| Salinivibrio | 23 | MEDKKVWISIGADAQQTALSSGAQPLLAQSVAHNGQAWIGEVSESELAA |
| spYCSC6 | | LSHEMHENHHRCGGYIVHSSAQSAMAASNMPLSRASFIAPAISQQALVTP |
| Aminopeptidase | | WISQIDSALIVNTIDRLTDFPNRFYTTTSGAQASDWIKQRWQSLSAGLAGA |
| | | SVTQISHSGYNQASVMLTIEGSESPDEWVVVGGHLDSTIGSRTNEQSIAPG |
| | | ADDDASGIAAVTEVIRVLAQNNFQPKRSIAFVAYAAEEVGLRGSQDVAN |
| | | QFKQAGKDVRGVLQLDMTNYQGSAEDIVFITDYTDNQLTQYLTQLLDEY |
| | | LPTLNYGFDTCGYACSDHASWHQVGYPAAMPFEAKFNDYNPNIHTPQDT |
| | | LANSDSEGAHAAKFTKLGLAYTVELANADSSPNPGNELKLGEPINGLSGA |
| | | RGNEKYFNYRLDQSGELVIRTYGGSGDVDLYVKANGDVSTGNWDCRPY |
| | | RSGNDEVCRFDNATPGNYAVMLRGYRTYDNVSLIVEHHHHHH |
| Vibrio proteolyticus | 24 | GMPPITQQATVTAWLPQVDASQITGTISSLESFTNRFYTTTSGAQASDWIA |
| Aminopeptidase I | | SEWQ [symbol] LSASLPNASVKQVSHSGYNQKSVVMTITGSEAPDEWIVIGGHLDS |
| | | TIGSHTNEQSVAPGADDDASGIAAVTEVIRVLSENNFQPKRSIAFMAYAAE |
| | | EVGLRGSQDLANQYKSEGKNVVSALQLDMTNYKGSAQDVVFITDYTDS |
| | | NFTQYLTQLMDEYLPSLTYGFDTCGYACSDHASWHNAGYPAAMPFESKF |
| | | NDYNPRIHTTQDTLANSDPTGSHAKKFTQLGLAYAIEMGSATGDTPTPGN |
| | | QLEHHHHHH |
| P. furiosus | 25 | MVDWELMKKIIESPGVSGYEHLGIRDLVVDILKDVADEVKIDKLGNVIAH |
| Aminopeptidase I | | FKGSAPKVMVAAHMDKIGLMVNHIDKDGYLRVVPIGGVLPETLIAQKIRF |
| | | FTEKGERYGVVGVLPPHLRREAKDQGGKIDWDSIIVDVGASSREEAEEMG |
| | | FRIGTIGEFAPNFTRLSEHRFATPYLDDRICLYAMIEAARQLGEHEADIYIV |
| | | ASVQEEIGLRGARVASFAIDPEVGIAMDVTFAKQPNDKGKIVPELGKGPV |
| | | MDVGPNINPKLRQFADEVAKKYEIPLQVEPSPRPTGTDANVMQINREGVA |
| | | TAVLSIPIRYMHSQVELADARDVDNTIKLAKALLEELKPMDFTPLEHHHH |
| | | HH |

*Cleavage efficiency (from most to least): arginine > lysine > hydrophobic residues (including alanine, leucine, methionine, and phenylalanine) > proline (see, e.g., Matthews Biochemistry 47, 2008, 5303-5311).
**Cleavage efficiency (from most to least): leucine > alanine > arginine > phenylalanine > proline; does not cleave after glutamate and aspartate.

For the purposes of comparing two or more amino acid sequences, the percentage of “sequence identity” between a first amino acid sequence and a second amino acid sequence (also referred to herein as “amino acid identity”) may be calculated by dividing [the number of amino acid residues in the first amino acid sequence that are identical to the amino acid residues at the corresponding positions in the second amino acid sequence] by [the total number of amino acid residues in the first amino acid sequence] and multiplying by [100], in which each deletion, insertion, substitution or addition of an amino acid residue in the second amino acid sequence compared to the first amino acid sequence is considered as a difference at a single amino acid residue (position). Alternatively, the degree of sequence identity between two amino acid sequences may be calculated using a known computer algorithm (e.g., by the local homology algorithm of Smith and Waterman (1970) Adv. Appl. Math. 2:482c, by the homology alignment algorithm of Needleman and Wunsch, J. Mol. Biol. (1970) 48:443, by the search for similarity method of Pearson and Lipman. Proc. Natl. Acad. Sci. USA (1998) 85:2444, or by computerized implementations of algorithms available as Blast, Clustal Omega, or other sequence alignment algorithms) and, for example, using standard settings. Usually, for the purpose of determining the percentage of “sequence identity” between two amino acid sequences in accordance with the calculation method outlined hereinabove, the amino acid sequence with the greatest number of amino acid residues will be taken as the “first” amino acid sequence, and the other amino acid sequence will be taken as the “second” amino acid sequence.

Additionally, or alternatively, two or more sequences may be assessed for the identity between the sequences. The terms “identical” or percent “identity” in the context of two or more nucleic acids or amino acid sequences, refer to two or more sequences or subsequences that are the same. Two sequences are “substantially identical” if two sequences have a specified percentage of amino acid residues or nucleotides that are the same (e.g., at least 80%, 85%, 90%, 95%, 96%, 97%, 98%, 99%, 99.5%, 99.6%, 99.7%, 99.8%, or 99.9% identical) over a specified region or over the entire sequence, when compared and aligned for maximum correspondence over a comparison window, or designated region as measured using one of the above sequence comparison algorithms or by manual alignment and visual inspection. Optionally, the identity exists over a region that is at least about 25, 50, 75, or 100 amino acids in length, or over a region that is 100 to 150, 150 to 200, 100 to 200, or 200 or more, amino acids in length.

Additionally, or alternatively, two or more sequences may be assessed for the alignment between the sequences. The terms “alignment” or percent “alignment” in the context of two or more nucleic acids or amino acid sequences, refer to two or more sequences or subsequences that are the same. Two sequences are “substantially aligned” if two sequences have a specified percentage of amino acid residues or nucleotides that are the same (e.g., at least 80%, 85%, 90%, 95%, 96%, 97%, 98%, 99%, 99.5%, 99.6%, 99.7%, 99.8% or 99.9% identical) over a specified region or over the entire sequence, when compared and aligned for maximum correspondence over a comparison window, or designated region as measured using one of the above sequence comparison algorithms or by manual alignment and visual inspection. Optionally, the alignment exists over a region that is at least about 25, 50, 75, or 100 amino acids in length, or over a region that is 100 to 150, 150 to 200, 100 to 200, or 200 or more amino acids in length.

In addition to polypeptide molecules, nucleic acid molecules possess a variety of advantageous properties for use as affinity reagents (e.g., amino acid recognition molecules) in accordance with the application.

Nucleic acid aptamers are nucleic acid molecules that have been engineered to bind desired targets with high affinity and selectivity. Accordingly, nucleic acid aptamers may be engineered to selectively bind a desired type of amino acid using selection and/or enrichment techniques known in the art. Thus, in some embodiments, an affinity reagent comprises a nucleic acid aptamer (e.g., a DNA aptamer, an RNA aptamer). In some embodiments, a labeled affinity reagent is a labeled aptamer that selectively binds one type of terminal amino acid. For example, in some embodiments, labeled aptamer selectively binds one type of amino acid (e.g., a single type of amino acid or a subset of types of amino acids) at a terminus of a polypeptide, as described herein. Although not shown, it should be appreciated that labeled aptamer may be engineered to selectively bind one type of amino acid at any position of a polypeptide (e.g., at a terminal position or at terminal and internal positions of a polypeptide) in accordance with a method of the application.

In some embodiments, a labeled affinity reagent comprises a label having binding-induced luminescence. For example, in some embodiments, a labeled aptamer comprises a donor label and an acceptor label and functions. In yet other embodiments, labeled aptamer comprises a quenching moiety and functions analogously to a molecular beacon, wherein luminescence of labeled aptamer is internally quenched as a free molecule and restored as a selectively bound molecule (see, e.g., Hamaguchi, et al. (2001) Analytical Biochemistry 294, 126-131). Without wishing to be bound by theory, it is thought that these and other types of mechanisms for binding-induced luminescence may advantageously reduce or eliminate background luminescence to increase overall sensitivity and accuracy of the methods described herein.

In addition to methods of identifying a terminal amino acid of a polypeptide, the application provides methods of sequencing polypeptides using labeled affinity reagents. In some embodiments, methods of sequencing may involve subjecting a polypeptide terminus to repeated cycles of terminal amino acid detection and terminal amino acid cleavage. For example, in some embodiments, the application provides a method of determining an amino acid sequence of a polypeptide comprising contacting a polypeptide with one or more labeled affinity reagents described herein and subjecting the polypeptide to Edman degradation.

Conventional Edman degradation involves repeated cycles of modifying and cleaving the terminal amino acid of a polypeptide, wherein each successively cleaved amino acid is identified to determine an amino acid sequence of the polypeptide. As an illustrative example of a conventional Edman degradation, the N-terminal amino acid of a polypeptide is modified using phenyl isothiocyanate (PITC) to form a PITC-derivatized N-terminal amino acid. The PITC-derivatized N-terminal amino acid is then cleaved using acidic conditions, basic conditions, and/or elevated temperatures. It has also been shown that the step of cleaving the PITC-derivatized N-terminal amino acid may be accomplished enzymatically using a modified cysteine protease from the protozoa Trypanosoma cruzi, which involves relatively milder cleavage conditions at a neutral or near-neutral pH. Non-limiting examples of useful enzymes are described in U.S. patent application Ser. No. 15/255,433, filed Sep. 2, 2016, titled “MOLECULES AND METHODS FOR ITERATIVE POLYPEPTIDE ANALYSIS AND PROCESSING”.

In some embodiments, sequencing by Edman degradation comprises providing a polypeptide that is immobilized to a surface of a solid support (e.g., immobilized to a bottom or sidewall surface of a sample well) through a linker. In some embodiments, as described herein, polypeptide is immobilized at one terminus (e.g., an amino-terminal amino acid or a carboxy-terminal amino acid) such that the other terminus is free for detecting and cleaving of a terminal amino acid. Accordingly, in some embodiments, the reagents used in Edman degradation methods described herein preferentially interact with terminal amino acids at the non-immobilized (e.g., free) terminus of polypeptide. In this way, polypeptide remains immobilized over repeated cycles of detecting and cleaving. To this end, in some embodiments, linker may be designed according to a desired set of conditions used for detecting and cleaving, e.g., to limit detachment of polypeptide from surface under chemical cleavage conditions. Suitable linker compositions and techniques for immobilizing a polypeptide to a surface are described in detail elsewhere herein.

In accordance with the application, in some embodiments, a method of sequencing by Edman degradation comprises a step (i) of contacting a polypeptide with one or more labeled affinity reagents that selectively bind one or more types of terminal amino acids. In some embodiments, a labeled affinity reagent interacts with the polypeptide by selectively binding the terminal amino acid. In some embodiments, step (i) further comprises removing any of the one or more labeled affinity reagents that do not selectively bind the terminal amino acid (e.g., the free terminal amino acid) of polypeptide.

In some embodiments, the method further comprises identifying the terminal amino acid of the polypeptide by detecting labeled affinity reagent. In some embodiments, detecting comprises detecting a luminescence from labeled affinity reagent. As described herein, in some embodiments, the luminescence is uniquely associated with labeled affinity reagent, and the luminescence is thereby associated with the type of amino acid to which labeled affinity reagent selectively binds. As such, in some embodiments, the type of amino acid is identified by determining one or more luminescence properties of labeled affinity reagent.

In some embodiments, a method of sequencing by Edman degradation comprises a step (ii) of removing the terminal amino acid of the polypeptide. In some embodiments, step (ii) comprises removing labeled affinity reagent (e.g., any of the one or more labeled affinity reagents that selectively bind the terminal amino acid) from the polypeptide. In some embodiments, step (ii) comprises modifying the terminal amino acid (e.g., the free terminal amino acid) of the polypeptide by contacting the terminal amino acid with an isothiocyanate (e.g., PITC) to form an isothiocyanate-modified terminal amino acid. In some embodiments, an isothiocyanate-modified terminal amino acid is more susceptible to removal by a cleaving reagent (e.g., a chemical or enzymatic cleaving reagent) than an unmodified terminal amino acid.

In some embodiments, step (ii) comprises removing the terminal amino acid by contacting the polypeptide with a protease that specifically binds and cleaves the isothiocyanate-modified terminal amino acid. In some embodiments, the protease comprises a modified cysteine protease. In some embodiments, the protease comprises a modified cysteine protease, such as a cysteine protease from Trypanosoma cruzi (see, e.g., Borgo, et al. (2015) Protein Science 24:571-579). In yet other embodiments, step (ii) comprises removing the terminal amino acid by subjecting the polypeptide to chemical (e.g., acidic, basic) conditions sufficient to cleave the isothiocyanate-modified terminal amino acid.

In some embodiments, a method of sequencing by Edman degradation comprises a step (iii) of washing the polypeptide following terminal amino acid cleavage. In some embodiments, washing comprises removing the protease. In some embodiments, washing comprises restoring the polypeptide to neutral pH conditions (e.g., following chemical cleavage by acidic or basic conditions). In some embodiments, a method of sequencing by Edman degradation comprises repeating steps (i) through (iii) for a plurality of cycles.

In some embodiments, a sample containing a complex mixture or enriched mixture of polypeptides (e.g., a mixture of polypeptides) can be degraded using common enzymes into short polypeptide fragments of approximately 6 to 40 amino acids. In some embodiments, sequencing of this polypeptide library in accordance with methods of the application would reveal the identity and abundance of each of the polypeptides present in the original complex mixture or enriched mixture. As described herein and in the literature, most polypeptides in the size range of 6 to 40 amino acids can be uniquely identified by determining the number and location of just four amino acids within a polypeptide chain.

Accordingly, in some embodiments, a method of sequencing by Edman degradation may be performed using a set of labeled aptamers comprising four DNA aptamer types, each type recognizing a different N-terminal amino acid. Each aptamer type may be labeled with a different luminescent label, such that the different aptamer types can be distinguished based on one or more luminescence properties. For illustrative purposes, the example set of labeled aptamers includes: a cysteine-specific aptamer labeled with a first luminescent label (“dye 1”); a lysine-specific aptamer labeled with a second luminescent label (“dye 2”); a tryptophan-specific aptamer labeled with a third luminescent label (“dye 3”); and a glutamate-specific aptamer labeled with a fourth luminescent label (“dye 4”).

In some embodiments, prior to step (i), single polypeptide molecules from a polypeptide library are immobilized to a surface of a solid support, e.g., at a bottom or sidewall surface of a sample well of an array of sample wells. In some embodiments, as described elsewhere herein, moieties that enable surface immobilization (e.g., biotin) or improve solubility (e.g., oligonucleotides) may be chemically or enzymatically attached to the C-terminus of the polypeptides. To determine the sequence of each polypeptide, in some embodiments, immobilized polypeptides are subjected to repeated cycles of N-terminal amino acid detection and N-terminal amino acid cleavage. In some embodiments, the process comprises reagent addition and wash steps which are performed by injection into a flowcell above the detection surface using an automated fluidic system. In some embodiments, steps (i) through (iv) illustrate one cycle of detection and cleavage using labeled aptamers.

In some embodiments, a method of sequencing by Edman degradation comprises a step (i) of flowing in a mixture of four orthogonally labeled DNA aptamers and incubating to allow the aptamers to bind to any immobilized polypeptides (e.g., polypeptides immobilized within a sample well of an array) that contain one of the four correct amino acids at the N-terminus. In some embodiments, the method further comprises washing the immobilized polypeptides to remove unbound aptamers. In some embodiments, the method further comprises imaging the immobilized polypeptides (“Imaging step (i)”). In some embodiments, the acquired images contain enough information to determine the location of aptamer-bound polypeptides (e.g., location within an array of sample wells) and which of the four aptamers is bound at each location. In some embodiments, the method further comprises washing the immobilized polypeptides using an appropriate buffer to remove the aptamers from the immobilized polypeptides.

In some embodiments, a method of sequencing comprises a step (ii) of flowing in a solution containing a reactive molecule (e.g., PITC, as shown) that specifically modifies the N-terminal amine group. An isothiocyanate molecule such as PITC, in some embodiments, modifies the N-terminal amino acid into a substrate for cleavage by a modified protease such as the cysteine protease cruzain from Trypanosoma Cruzi.

In some embodiments, a method of sequencing according comprises a step (iii) of washing the immobilized polypeptides before flowing in a suitable modified protease that recognizes and cleaves the modified N-terminal amino acid from the immobilized polypeptide.

In some embodiments, the method comprises a step (iv) of washing the immobilized polypeptides after enzymatic cleavage. In some embodiments, steps (i) through (iv) depict one cycle of Edman degradation. Accordingly, step (i′) as shown is the start of the next reaction cycle which proceeds as steps (i′) through (iv′) performed as described above for steps (i) through (iv). In some embodiments, steps (i) through (iv) are repeated for approximately 20-40 cycles.

In some embodiments, a labeled isothiocyanate (e.g., a dye-labeled PITC) may be used to monitor sample loading. For example, in some embodiments, prior to subjecting a polypeptide sample to a method of sequencing, the polypeptide sample is pre-conjugated with a luminescent label at a terminal end by modification of the terminal end using a dye-labeled PITC. In this way, loading of the polypeptide sample into an array of sample wells may be monitored by detecting luminescence from the labels prior to step (i) described above. In some embodiments, the luminescence is used to determine single occupancy of sample wells in the array (e.g., a fraction of sample wells containing a single polypeptide molecule), which may advantageously increase the amount of information reliably obtained for a given sample. Once a desired sample loading status is determined by luminescence, chemical or enzymatic cleavage may be performed, as described, before proceeding with step (i).

In some embodiments, a labeled isothiocyanate (e.g., a dye-labeled PITC) may be used to monitor reaction progress for a polypeptide sample in an array. For example, in some embodiments, step (ii) comprises flowing in a solution containing a dye-labeled PITC that specifically modifies and labels N-terminal amine groups of polypeptides in the sample. In some embodiments, luminescence from the labels may be detected during or after step (ii) to evaluate N-terminal PITC modification of polypeptides in the sample. Accordingly, in some embodiments, luminescence is used to determine whether or when to proceed from step (ii) to step (iii). In some embodiments, luminescence from the labels may be detected during or after step (iii) to evaluate N-terminal amino acid cleavage of polypeptides in the sample—e.g., to determine whether or when to proceed from step (iii) to step (iv).

A method of sequencing may utilize separate reagents for detecting and cleaving a terminal amino acid of a polypeptide. Nonetheless, in some aspects, the application provides a method of sequencing in which a single reagent comprising a peptidase (such as a labeled exopeptidase that selectively binds and cleaves a different type of terminal amino acid) may be used for detecting and cleaving a terminal amino acid of a polypeptide.

Labeled exopeptidases may comprise a lysine-specific exopeptidase comprising a first luminescent label, a glycine-specific exopeptidase comprising a second luminescent label, an aspartate-specific exopeptidase comprising a third luminescent label, and a leucine-specific exopeptidase comprising a fourth luminescent label. In accordance with certain embodiments described herein, each of labeled exopeptidases selectively binds and cleaves its respective amino acid only when that amino acid is at an amino- or carboxy-terminus of a polypeptide. Accordingly, as sequencing by this approach proceeds from one terminus of a peptide toward the other, labeled exopeptidases are engineered or selected such that all reagents of the set will possess either aminopeptidase or carboxypeptidase activity.

In some aspects, the application provides methods of polypeptide sequencing in real-time by evaluating binding interactions of terminal amino acids with labeled amino acid recognition molecules (e.g., labeled affinity reagents) and a labeled cleaving reagent (e.g., a labeled non-specific exopeptidase). Without wishing to be bound by theory, a labeled affinity reagent selectively binds according to a binding affinity (KD) defined by an association rate, or an “on” rate, of binding (kₒₙ) and a dissociation rate, or an “off” rate, of binding (k_{off}). The rate constants k_{off} and kₒₙ are the critical determinants of pulse duration (e.g., the time corresponding to a detectable binding event) and interpulse duration (e.g., the time between detectable binding events), respectively. In some embodiments, these rates can be engineered to achieve pulse durations and pulse rates (e.g., the frequency of signal pulses) that give the best sequencing accuracy.

A sequencing reaction mixture may further comprise a labeled non-specific exopeptidase comprising a luminescent label that is different than that of labeled affinity reagent. In some embodiments, a labeled non-specific exopeptidase is present in the mixture at a concentration that is less than that of the labeled affinity reagent. In some embodiments, the labeled non-specific exopeptidase displays broad specificity such that it cleaves most or all types of terminal amino acids.

In some embodiments, terminal amino acid cleavage by a labeled non-specific exopeptidase gives rise to a signal pulse, and these events occur with lower frequency than the binding pulses of a labeled affinity reagent. In this way, amino acids of a polypeptide may be counted and/or identified in a real-time sequencing process. In some embodiments, a plurality of labeled affinity reagents may be used, each with a diagnostic pulsing pattern (e.g., characteristic pattern) which may be used to identify a corresponding terminal amino acid. For example, in some embodiments, different characteristic patterns correspond to the association of more than one labeled affinity reagent with different types of terminal amino acids. As described herein, it should be appreciated that a single affinity reagent that associates with more than one type of amino acid may be used in accordance with the application. Accordingly, in some embodiments, different characteristic patterns correspond to the association of one labeled affinity reagent with different types of terminal amino acids.

As detailed above, a real-time sequencing process can generally involve cycles of terminal amino acid recognition and terminal amino acid cleavage, where the relative occurrence of recognition and cleavage can be controlled by a concentration differential between a labeled affinity reagent and a labeled non-specific exopeptidase. In some embodiments, the concentration differential can be optimized such that the number of signal pulses detected during recognition of an individual amino acid provides a desired confidence interval for identification. For example, if an initial sequencing reaction provides signal data with too few signal pulses between cleavage events to permit determination of characteristic patterns with a desired confidence interval, the sequencing reaction can be repeated using a decreased concentration of non-specific exopeptidase relative to affinity reagent. The inventors have recognized further techniques for controlling real-time sequencing reactions, which may be used in combination with, or alternatively to, the concentration differential approach as described.

In some embodiments, a sequencing reaction involves cycles of temperature-dependent terminal amino acid recognition and terminal amino acid cleavage. Each cycle of the sequencing reaction may be carried out over two temperature ranges: a first temperature range (“T₁”) that is optimal for affinity reagent activity over exopeptidase activity (e.g., to promote terminal amino acid recognition), and a second temperature range (“T₂”) that is optimal for exopeptidase activity over affinity reagent activity (e.g., to promote terminal amino acid cleavage). The sequencing reaction may progress by alternating the reaction mixture temperature between the first temperature range T₁ (to initiate amino acid recognition) and the second temperature range T₂ (to initiate amino acid cleavage). Accordingly, progression of a temperature-dependent sequencing process is controllable by temperature, and alternating between different temperature ranges (e.g., between T₁ and T₂) which may be carried through manual or automated processes. In some embodiments, affinity reagent activity (e.g., binding affinity (K_{D}) for an amino acid) within the first temperature range T₁ as compared to the second temperature range T₂ is increased by at least 10-fold, at least 100-fold, at least 1,000-fold, at least 10,000-fold, at least 100,000-fold, or more. In some embodiments, exopeptidase activity (e.g., rate of substrate conversion to cleavage product) within the second temperature range T₂ as compared to the first temperature range T₁ is increased by at least 2-fold, 10-fold, at least 25-fold, at least 50-fold, at least 100-fold, at least 1,000-fold, or more.

In some embodiments, the first temperature range T₁ is lower than the second temperature range T₂. In some embodiments, the first temperature range T₁ is between about 15° C. and about 40° C. (e.g., between about 25° C. and about 35° C., between about 15° C. and about 30° C., between about 20° C. and about 30° C.). In some embodiments, the second temperature range T₂ is between about 40° C. and about 100° C. (e.g., between about 50° C. and about 90° C., between about 60° C. and about 90° C., between about 70° C. and about 90° C.). In some embodiments, the first temperature range T₁ is between about 20° C. and about 40° C. (e.g., approximately 30° C.), and the second temperature range T₂ is between about 60° C. and about 100° C. (e.g., approximately 80° C.).

In some embodiments, the first temperature range T₁ is higher than the second temperature range T₂. In some embodiments, the first temperature range T₁ is between about 40° C. and about 100° C. (e.g., between about 50° C. and about 90° C., between about 60° C. and about 90° C., between about 70° C. and about 90° C.). In some embodiments, the second temperature range T₂ is between about 15° C. and about 40° C. (e.g., between about 25° C. and about 35° C., between about 15° C. and about 30° C., between about 20° C. and about 30° C.). In some embodiments, the first temperature range T₁ is between about 60° C. and about 100° C. (e.g., approximately 80° C.), and the second temperature range T₂ is between about 20° C. and about 40° C. (e.g., approximately 30° C.).

In some embodiments, the application provides a luminescence-dependent sequencing process using luminescence-activated reagents. In some embodiments, a luminescence-dependent sequencing process involves cycles of luminescence-dependent amino acid recognition and cleavage. Each cycle of the sequencing reaction may be carried out by exposing a sequencing reaction mixture to two different luminescent conditions: a first luminescent condition that is optimal for affinity reagent activity over exopeptidase activity (e.g., to promote amino acid recognition), and a second luminescent condition that is optimal for exopeptidase activity over affinity reagent activity (e.g., to promote amino acid cleavage). The sequencing reaction progresses by alternating between exposing the reaction mixture to the first luminescent condition (to initiate amino acid recognition) and exposing the reaction mixture to the second luminescent condition (to initiate amino acid cleavage). By way of example and not limitation, in some embodiments, the two different luminescent conditions comprise a first wavelength and a second wavelength.

In some aspects, the application provides methods of polypeptide sequencing in real-time by evaluating binding interactions of one or more labeled affinity reagents with terminal and internal amino acids and binding interactions of a labeled non-specific exopeptidase with terminal amino acids. In some embodiments, a labeled affinity reagent is used that selectively binds to and dissociates from one type of amino acid at both terminal and internal positions. The selective binding gives rise to a series of pulses in signal output. In this approach, however, the series of pulses occur at a rate that is determined by the number of the type of amino acid throughout the polypeptide. Accordingly, in some embodiments, the rate of pulsing corresponding to binding events would be diagnostic of the number of cognate amino acids currently present in the polypeptide.

A labeled non-specific peptidase may be present at a relatively lower concentration than the labeled affinity reagent, e.g., to give optimal time windows in between cleavage events. Additionally, in certain embodiments, uniquely identifiable luminescent label of labeled non-specific peptidase would indicate when cleavage events have occurred. As the polypeptide undergoes iterative cleavage, the rate of pulsing corresponding to binding by the labeled affinity reagent would drop in a step-wise manner whenever a terminal amino acid is cleaved by the labeled non-specific peptidase. Thus, in some embodiments, amino acids may be identified—and polypeptides thereby sequenced—in this approach based on a pulsing pattern and/or on the rate of pulsing that occurs within a pattern detected between cleavage events.

### B. Sequencing by Degradation of Labeled Polypeptides

In some aspects, the application provides methods of sequencing a polypeptide by identifying a unique combination of amino acids corresponding to a known polypeptide sequence. In some embodiments, the method comprises detecting selectively labeled amino acids of a labeled polypeptide. In some embodiments, the labeled polypeptide comprises selectively modified amino acids such that different amino acid types comprise different luminescent labels. As used herein, unless otherwise indicated, a labeled polypeptide refers to a polypeptide comprising one or more selectively labeled amino acid sidechains. Methods of selective labeling and details relating to the preparation and analysis of labeled polypeptides are known in the art (see, e.g., Swaminathan, et al. PLoS Comput Biol. 2015, 11(2):e1004080).

As described herein, in some aspects, the application provides methods of sequencing a polypeptide by obtaining data during a polypeptide degradation process, and analyzing the data to determine portions of the data corresponding to amino acids that are sequentially exposed at a terminus of the polypeptide during the degradation process. In some embodiments, the portions of the data comprise a series of signal pulses indicative of association of one or more amino acid recognition molecules with successive amino acids exposed at the terminus of the polypeptide (e.g., during a degradation). In some embodiments, the series of signal pulses corresponds to a series of reversible single molecule binding interactions at the terminus of the polypeptide during the degradation process.

In some aspects, the polypeptide sequencing techniques described herein generate data indicating how a polypeptide interacts with a binding means (e.g., one or more amino acid recognition molecules) while the polypeptide is being degraded by a cleaving means (e.g., one or more cleaving reagents). As discussed above, the data can include a series of characteristic patterns corresponding to association events at a terminus of a polypeptide in between cleavage events at the terminus. In some embodiments, methods of sequencing described herein comprise contacting a single polypeptide molecule with a binding means and a cleaving means, where the binding means and the cleaving means are configured to achieve at least 10 association events prior to a cleavage event. In some embodiments, the means are configured to achieve the at least 10 association events between two cleavage events.

As described herein, in some embodiments, a plurality of single-molecule sequencing reactions are performed in parallel in an array of sample wells. In some embodiments, an array comprises between about 10,000 and about 1,000,000 sample wells. The volume of a sample well may be between about 10⁻²¹ liters and about 10⁻¹⁵ liters, in some implementations. Because the sample well has a small volume, detection of single-molecule events may be possible as only about one polypeptide may be within a sample well at any given time. Statistically, some sample wells may not contain a single-molecule sequencing reaction and some may contain more than one single polypeptide molecule. However, an appreciable number of sample wells may each contain a single-molecule reaction (e.g., at least 30% in some embodiments), so that single-molecule analysis can be carried out in parallel for a large number of sample wells. In some embodiments, the binding means and the cleaving means are configured to achieve at least 10 association events prior to a cleavage event in at least 10% (e.g., 10-50%, more than 50%, 25-75%, at least 80%, or more) of the sample wells in which a single-molecule reaction is occurring. In some embodiments, the binding means and the cleaving means are configured to achieve at least 10 association events prior to a cleavage event for at least 50% (e.g., more than 50%, 50-75%, at least 80%, or more) of the amino acids of a polypeptide in a single-molecule reaction.

In some embodiments, a labeled polypeptide is immobilized and exposed to an excitation source. An aggregate luminescence from the labeled polypeptide may be detected and, in some embodiments, exposure to luminescence over time may result in a loss in detected signal due to luminescent label degradation (e.g., degradation due to photobleaching). In some embodiments, the labeled polypeptide comprises a unique combination of selectively labeled amino acids that give rise to an initial detected signal. Degradation of luminescent labels over time results in a corresponding decrease in a detected signal for the photobleached labeled polypeptide. In some embodiments, the signal can be deconvoluted by analysis of one or more luminescence properties (e.g., signal deconvolution by luminescence lifetime analysis). In some embodiments, the unique combination of selectively labeled amino acids of the labeled polypeptide have been computationally precomputed and empirically verified—e.g., based on known polypeptide sequences of a proteome. In some embodiments, the combination of detected amino acid labels are compared against a database of known sequences of a proteome of an organism to identify a particular polypeptide of the database corresponding to the labeled polypeptide.

In some embodiments, an optimal sample concentration is determined for performing a sequencing reaction that maximizes sampling in massively parallel analysis. In some embodiments, the concentration is selected so that a desired fraction of the sample wells of an array (e.g., 30%) are occupied at any given time. Without wishing to be bound by theory, it is thought that while a polypeptide is bleached over a period of time, the same well continues to be available for further analysis. Through diffusion, approximately 30% of the sample wells of an array can be used for analysis every 3 minutes. As an illustrative example, in a million sample well chip, 6,000,000 polypeptides per hour may be sampled, or 24,000,000 over a 4 hour period.

In some aspects, the application provides a method of sequencing a polypeptide by detecting luminescence of a labeled polypeptide which is subjected to repeated cycles of terminal amino acid modification and cleavage. In some embodiments, the method generally proceeds as described herein for other methods of sequencing by Edman degradation.

In some embodiments, the method comprises a step of (i) modifying the terminal amino acid of a labeled polypeptide. As described elsewhere herein, in some embodiments, modifying comprises contacting the terminal amino acid with an isothiocyanate (e.g., PITC) to form an isothiocyanate-modified terminal amino acid. In some embodiments, an isothiocyanate modification converts the terminal amino acid to a form that is more susceptible to removal by a cleaving reagent (e.g., a chemical or enzymatic cleaving reagent, as described herein). Accordingly, in some embodiments, the method comprises a step of (ii) removing the modified terminal amino acid using chemical or enzymatic means detailed elsewhere herein for Edman degradation.

In some embodiments, the method comprises repeating steps (i) through (ii) for a plurality of cycles, during which luminescence of the labeled polypeptide is detected, and cleavage events corresponding to the removal of a labeled amino acid from the terminus may be detected as a decrease in detected signal. In some embodiments, no change in signal following step (ii) identifies an amino acid of unknown type. Accordingly, in some embodiments, partial sequence information may be determined by evaluating a signal detected following step (ii) during each sequential round by assigning an amino acid type by a determined identity based on a change in detected signal or identifying an amino acid type as unknown based on no change in a detected signal.

In some aspects, a method of sequencing a polypeptide in accordance with the application comprises sequencing by processive enzymatic cleavage of a labeled polypeptide. In some embodiments, a labeled polypeptide is subjected to degradation using a modified processive exopeptidase that continuously cleaves a terminal amino acid from one terminus to another terminus. Exopeptidases are described in detail elsewhere herein. In some embodiments, a labeled polypeptide is subjected to degradation by an immobilized processive exopeptidase. In some embodiments, an immobilized labeled polypeptide is subjected to degradation by a processive exopeptidase.

In some embodiments, the rate of processivity of processive exopeptidase is known, such that the timing between a detected decrease in signal may be used to calculate the number of unlabeled amino acids between each detection event. For example, if a polypeptide of 40 amino acids was cleaved in such a way that an amino acid was removed every second, a labeled polypeptide having 3 signals would show all 3 initially, then 2, then 1, and finally no signal. In this way, the order of the labeled amino acids can be determined. Accordingly, these methods may be used to determine partial sequence information, e.g., for proteomic analysis based on polypeptide fragment sequencing.

In some embodiments, single molecule polypeptide sequencing can be achieved using an ATP-based Förster resonance energy transfer (FRET) scheme (e.g., with one or more labeled cofactors). In some embodiments, sequencing by cofactor-based FRET can be performed using an immobilized ATP-dependent protease, donor-labeled ATP, and acceptor-labeled amino acids of a polypeptide substrate. In some embodiments, amino acids can be labeled with acceptors, and the one or more cofactors can be labeled with donors.

For example, in some embodiments, extracted polypeptides are denatured, and cysteines and lysines are labeled with fluorescent dyes. In some embodiments, an engineered version of a protein translocase (e.g., bacterial ClpX) is used to bind to individual substrate polypeptides, unfold them, and translocate them through its nano-channel. In some embodiments, the translocase is labeled with a donor dye, and FRET occurs between the donor on the translocase and two or more distinct acceptor dyes on a substrate when the substrate passes through the nano-channel. The order of the labeled amino acids can then be determined from the FRET signal. In some embodiments, one or more of the following non-limiting labeled ATP analogues shown in Table 3 can be used.

TABLE 3
Non-limiting examples of labeled ATP analogues
Phosphate-labeled ATP:

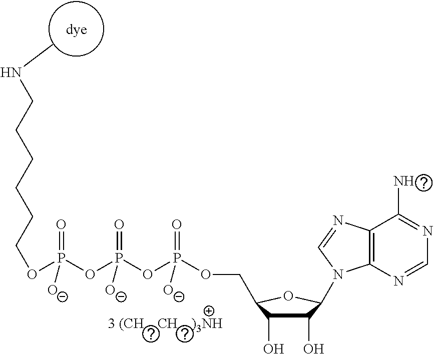

(γ-[(6-Amino)hexyl]-ATP)

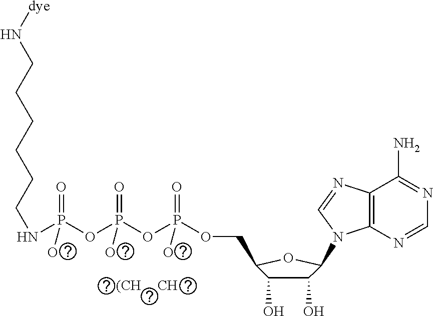

(γ-[(6-Aminohexyl)imido]-ATP)

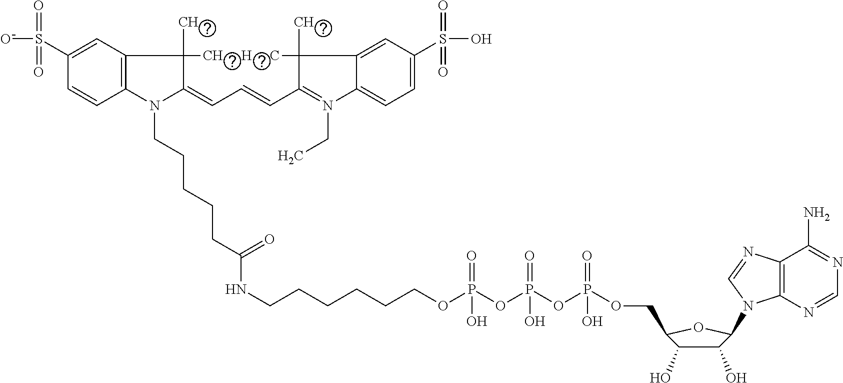

(γ-(6-Aminohexyl)-ATP—Cy3)

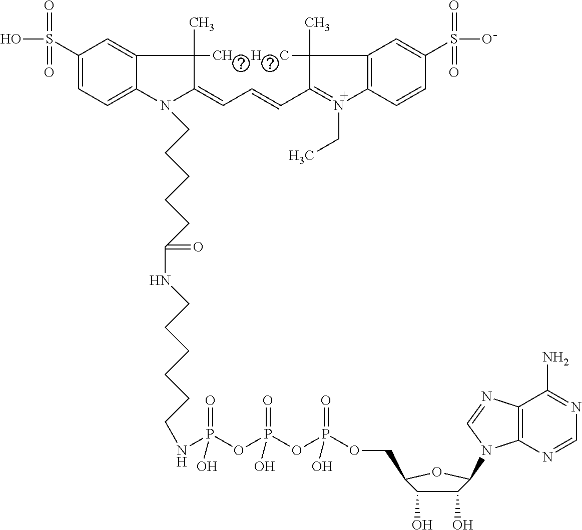

(γ-[(6-Aminohexyl)imido]-ATP—Cy3)

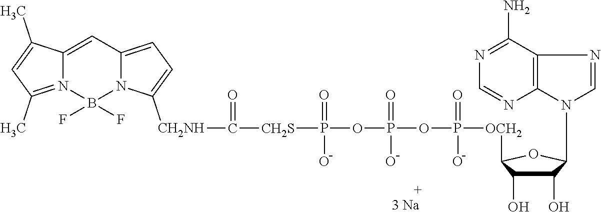

(BODIPY FL ATPγS)
Ribose-labeled ATP:

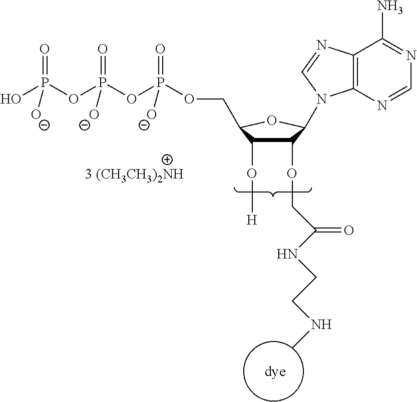

(EDA-ATP)

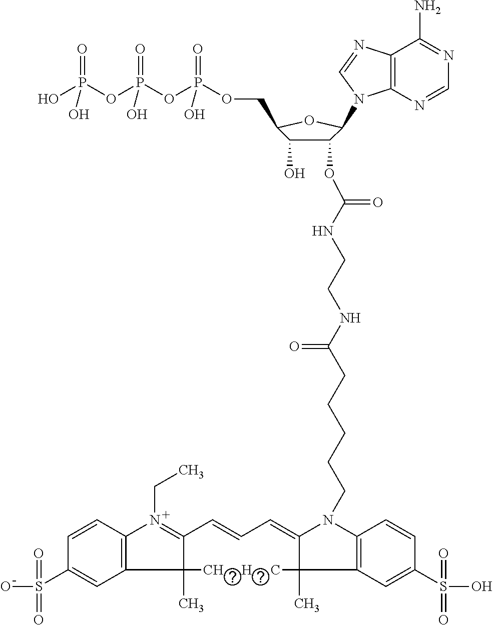

(EDA-ATP—Cy3)

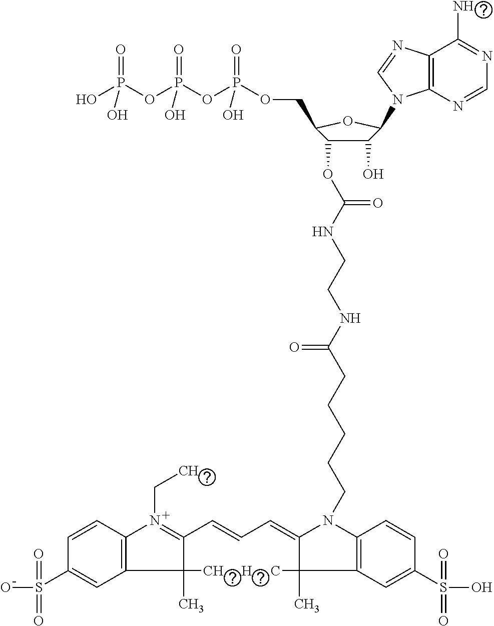

(EDA-ATP—Cy3)
Base-labeled ATP:

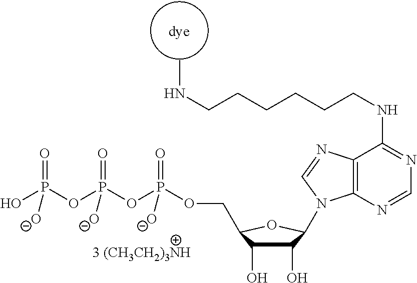

(N⁶-(6-Amino)hexyl-ATP)

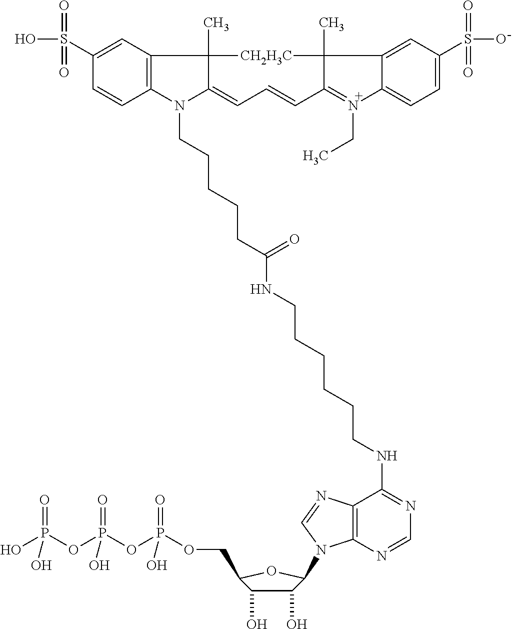

(N⁶-(6-Aminohexyl)-ATP—Cy3)

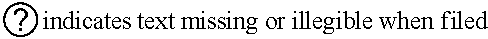

### C. Preparation of Samples for Sequencing

A polypeptide sample (e.g., an enriched polypeptide sample) can be modified prior to sequencing.

In some embodiments, the N-terminal amino acid or the C-terminal amino acid of a polypeptide is modified. In some embodiments, a terminal end of a polypeptides is modified with moieties that enable immobilization to a surface (e.g., a surface of a sample well on a chip used for polypeptide analysis). In some embodiments, such methods comprise modifying a terminal end of a labeled polypeptide to be analyzed in accordance with the application. In yet other embodiments, such methods comprise modifying a terminal end of a protein or enzyme that degrades or translocates a polypeptide substrate in accordance with the application.

In some embodiments, a carboxy-terminus of a polypeptide is modified in a method comprising: (i) blocking free carboxylate groups of the polypeptide; (ii) denaturing the polypeptide (e.g., by heat and/or chemical means); (iii) blocking free thiol groups of the polypeptide; (iv) digesting the polypeptide to produce at least one polypeptide fragment comprising a free C-terminal carboxylate group; and (v) conjugating (e.g., chemically) a functional moiety to the free C-terminal carboxylate group. In some embodiments, the method further comprises, after (i) and before (ii), dialyzing a sample comprising the polypeptide.

In some embodiments, a carboxy-terminus of a polypeptide is modified in a method comprising: (i) denaturing the polypeptide (e.g., by heat and/or chemical means); (ii) blocking free thiol groups of the polypeptide; (iii) digesting the polypeptide to produce at least one polypeptide fragment comprising a free C-terminal carboxylate group; (iv) blocking the free C-terminal carboxylate group to produce at least one polypeptide fragment comprising a blocked C-terminal carboxylate group; and (v) conjugating (e.g., enzymatically) a functional moiety to the blocked C-terminal carboxylate group. In some embodiments, the method further comprises, after (iv) and before (v), dialyzing a sample comprising the polypeptide.

In some embodiments, blocking free carboxylate groups refers to a chemical modification of these groups which alters chemical reactivity relative to an unmodified carboxylate. Suitable carboxylate blocking methods are known in the art and should modify side-chain carboxylate groups to be chemically different from a carboxy-terminal carboxylate group of a polypeptide to be functionalized. In some embodiments, blocking free carboxylate groups comprises esterification or amidation of free carboxylate groups of a polypeptide. In some embodiments, blocking free carboxylate groups comprises methyl esterification of free carboxylate groups of a polypeptide, e.g., by reacting the polypeptide with methanolic HCl. Additional examples of reagents and techniques useful for blocking free carboxylate groups include, without limitation, 4-sulfo-2,3,5,6-tetrafluorophenol (STP) and/or a carbodiimide such as N-(3-Dimethylaminopropyl)-N′-ethylcarbodiimide hydrochloride (EDAC), uronium reagents, diazomethane, alcohols and acid for Fischer esterification, the use of N-hydroxylsuccinimide (NHS) to form NHS esters (potentially as an intermediate to subsequent ester or amine formation), or reaction with carbonyldiimidazole (CDI) or the formation of mixed anhydrides, or any other method of modifying or blocking carboxylic acids, potentially through the formation of either esters or amides.

In some embodiments, blocking free thiol groups refers to a chemical modification of these groups which alters chemical reactivity relative to an unmodified thiol. In some embodiments, blocking free thiol groups comprises reducing and alkylating free thiol groups of a polypeptide. In some embodiments, reduction and alkylation is carried out by contacting a polypeptide with dithiothreitol (DTT) and one or both of iodoacetamide and iodoacetic acid. Examples of additional and alternative cysteine-reducing reagents which may be used are well known and include, without limitation, 2-mercaptoethanol, Tris (2-carboxyehtyl) phosphine hydrochloride (TCEP), tributylphosphine, dithiobutylamine (DTBA), or any reagent capable of reducing a thiol group. Examples of additional and alternative cysteine-blocking (e.g., cysteine-alkylating) reagents which may be used are well known and include, without limitation, acrylamide, 4-vinylpyridine, N-Ethylmalemide (NEM), N-ε-maleimidocaproic acid (EMCA), or any reagent that modifies cysteines so as to prevent disulfide bond formation.

In some embodiments, digestion comprises enzymatic digestion. In some embodiments, digestion is carried out by contacting a polypeptide with an endopeptidase (e.g., trypsin) under digestion conditions. In some embodiments, digestion comprises chemical digestion. Examples of suitable reagents for chemical and enzymatic digestion are known in the art and include, without limitation, trypsin, chemotrypsin, Lys-C, Arg-C, Asp-N, Lys-N, BNPS-Skatole, CNBr, caspase, formic acid, glutamyl endopeptidase, hydroxylamine, iodosobenzoic acid, neutrophil elastase, pepsin, proline-endopeptidase, proteinase K, staphylococcal peptidase I, thermolysin, and thrombin.

In some embodiments, the functional moiety comprises a biotin molecule. In some embodiments, the functional moiety comprises a reactive chemical moiety, such as an alkynyl. In some embodiments, conjugating a functional moiety comprises biotinylation of carboxy-terminal carboxy-methyl ester groups by carboxypeptidase Y, as known in the art.

In some embodiments, a solubilizing moiety is added to a polypeptide. Accordingly, in some embodiments methods and compositions provided herein are useful for modifying terminal ends of polypeptides with moieties that increase their solubility. In some embodiments, a solubilizing moiety is useful for small polypeptides that result from fragmentation (e.g., enzymatic fragmentation, for example using trypsin) and that are relatively insoluble. For example, in some embodiments, short polypeptides in a polypeptide pool can be solubilized by conjugating a polymer (e.g., a short oligo, a sugar, or other charged polymer) to the polypeptides.

### D. Luminescent Labels

As used herein, a luminescent label is a molecule that absorbs one or more photons and may subsequently emit one or more photons after one or more time durations. In some embodiments, the term is used interchangeably with “label” or “luminescent molecule” depending on context. A luminescent label in accordance with certain embodiments described herein may refer to a luminescent label of a labeled affinity reagent, a luminescent label of a labeled peptidase (e.g., a labeled exopeptidase, a labeled non-specific exopeptidase), a luminescent label of a labeled peptide, a luminescent label of a labeled cofactor, or another labeled composition described herein. In some embodiments, a luminescent label in accordance with the application refers to a labeled amino acid of a labeled polypeptide comprising one or more labeled amino acids.

In some embodiments, a luminescent label may comprise a first and second chromophore. In some embodiments, an excited state of the first chromophore is capable of relaxation via an energy transfer to the second chromophore. In some embodiments, the energy transfer is a Förster resonance energy transfer (FRET). Such a FRET pair may be useful for providing a luminescent label with properties that make the label easier to differentiate from amongst a plurality of luminescent labels in a mixture. In yet other embodiments, a FRET pair comprises a first chromophore of a first luminescent label and a second chromophore of a second luminescent label. In certain embodiments, the FRET pair may absorb excitation energy in a first spectral range and emit luminescence in a second spectral range.

In some embodiments, a luminescent label refers to a fluorophore or a dye. Typically, a luminescent label comprises an aromatic or heteroaromatic compound and can be a pyrene, anthracene, naphthalene, naphthylamine, acridine, stilbene, indole, benzindole, oxazole, carbazole, thiazole, benzothiazole, benzoxazole, phenanthridine, phenoxazine, porphyrin, quinoline, ethidium, benzamide, cyanine, carbocyanine, salicylate, anthranilate, coumarin, fluorescein, rhodamine, xanthene, or other like compound.

In some embodiments, a luminescent label comprises a dye selected from one or more of the following: 5/6-Carboxyrhodamine 6G, 5-Carboxyrhodamine 6G, 6-Carboxyrhodamine 6G, 6-TAMRA, Abberior® STAR 440SXP, Abberior® STAR 470SXP, Abberior® STAR 488, Abberior® STAR 512, Abberior® STAR 520SXP, Abberior® STAR 580, Abberior® STAR 600, Abberior® STAR 635, Abberior® STAR 635P, Abberior® STAR RED, Alexa Fluor® 350, Alexa Fluor® 405, Alexa Fluor® 430, Alexa Fluor® 480, Alexa Fluor® 488, Alexa Fluor® 514, Alexa Fluor® 532, Alexa Fluor® 546, Alexa Fluor® 555, Alexa Fluor® 568, Alexa Fluor® 594, Alexa Fluor® 610-X, Alexa Fluor® 633, Alexa Fluor® 647, Alexa Fluor® 660, Alexa Fluor® 680, Alexa Fluor® 700, Alexa Fluor® 750, Alexa Fluor® 790, AMCA, ATTO 390, ATTO 425, ATTO 465, ATTO 488, ATTO 495, ATTO 514, ATTO 520, ATTO 532, ATTO 542, ATTO 550, ATTO 565, ATTO 590, ATTO 610, ATTO 620, ATTO 633, ATTO 647, ATTO 647N, ATTO 655, ATTO 665, ATTO 680, ATTO 700, ATTO 725, ATTO 740, ATTO Oxa12, ATTO Rho101, ATTO Rho11, ATTO Rho12, ATTO Rho13, ATTO Rho14, ATTO Rho3B, ATTO Rho6G, ATTO Thio12, BD Horizon™ V450, BODIPY® 493/501, BODIPY® 530/550, BODIPY® 558/568, BODIPY® 564/570, BODIPY® 576/589, BODIPY® 581/591, BODIPY® 630/650, BODIPY® 650/665, BODIPY® FL, BODIPY® FL-X, BODIPY® R6G, BODIPY® TMR, BODIPY® TR, CAL Fluor® Gold 540, CAL Fluor® Green 510, CAL Fluor® Orange 560, CAL Fluor® Red 590, CAL Fluor® Red 610, CAL Fluor® Red 615, CAL Fluor® Red 635, Cascade® Blue, CF™350, CF™405M, CF™405S, CF™488A, CF™514, CF™532, CF™543, CF™546, CF™555, CF™568, CF™594, CF™620R, CF™633, CF™633-V1, CF™640R, CF™640R-V1, CF™640R-V2, CF™660C, CF™660R, CF™680, CF™680R, CF™680R-V1, CF™750, CF™770, CF™790, Chromeo™ 642, Chromis 425N, Chromis SOON, Chromis 515N, Chromis 530N, Chromis 550A, Chromis 550C, Chromis 550Z, Chromis 560N, Chromis 570N, Chromis 577N, Chromis 600N, Chromis 630N, Chromis 645A, Chromis 645C, Chromis 645Z, Chromis 678A, Chromis 678C, Chromis 678Z, Chromis 770A, Chromis 770C, Chromis 800A, Chromis 800C, Chromis 830A, Chromis 830C, Cy®3, Cy®3.5, Cy®3B, Cy®5, Cy®5.5, Cy®7, DyLight® 350, DyLight® 405, DyLight® 415-Col, DyLight® 425Q, DyLight® 485-LS, DyLight® 488, DyLight® 504Q, DyLight® 510-LS, DyLight® 515-LS, DyLight® 521-LS, DyLight® 530-R2, DyLight® 543Q, DyLight® 550, DyLight® 554-R0, DyLight® 554-R1, DyLight® 590-R2, DyLight® 594, DyLight® 610-B1, DyLight® 615-B2, DyLight® 633, DyLight® 633-B1, DyLight® 633-B2, DyLight® 650, DyLight® 655-B1, DyLight® 655-B2, DyLight® 655-B3, DyLight® 655-B4, DyLight® 662Q, DyLight® 675-B1, DyLight® 675-B2, DyLight® 675-B3, DyLight® 675-B4, DyLight® 679-05, DyLight® 680, DyLight® 683Q, DyLight® 690-B1, DyLight® 690-B2, DyLight® 696Q, DyLight® 700-B1, DyLight® 700-B1, DyLight® 730-B1, DyLight® 730-B2, DyLight® 730-B3, DyLight® 730-B4, DyLight® 747, DyLight® 747-B1, DyLight® 747-B2, DyLight® 747-B3, DyLight® 747-B4, DyLight® 755, DyLight® 766Q, DyLight® 775-B2, DyLight® 775-B3, DyLight® 775-B4, DyLight® 780-B1, DyLight® 780-B2, DyLight® 780-B3, DyLight® 800, DyLight® 830-B2, Dyomics-350, Dyomics-350XL, Dyomics-360XL, Dyomics-370XL, Dyomics-375XL, Dyomics-380XL, Dyomics-390XL, Dyomics-405, Dyomics-415, Dyomics-430, Dyomics-431, Dyomics-478, Dyomics-480XL, Dyomics-481XL, Dyomics-485XL, Dyomics-490, Dyomics-495, Dyomics-505, Dyomics-510XL, Dyomics-511XL, Dyomics-520XL, Dyomics-521XL, Dyomics-530, Dyomics-547, Dyomics-547P1, Dyomics-548, Dyomics-549, Dyomics-549P1, Dyomics-550, Dyomics-554, Dyomics-555, Dyomics-556, Dyomics-560, Dyomics-590, Dyomics-591, Dyomics-594, Dyomics-601XL, Dyomics-605, Dyomics-610, Dyomics-615, Dyomics-630, Dyomics-631, Dyomics-632, Dyomics-633, Dyomics-634, Dyomics-635, Dyomics-636, Dyomics-647, Dyomics-647P1, Dyomics-648, Dyomics-648P1, Dyomics-649, Dyomics-649P1, Dyomics-650, Dyomics-651, Dyomics-652, Dyomics-654, Dyomics-675, Dyomics-676, Dyomics-677, Dyomics-678, Dyomics-679P1, Dyomics-680, Dyomics-681, Dyomics-682, Dyomics-700, Dyomics-701, Dyomics-703, Dyomics-704, Dyomics-730, Dyomics-731, Dyomics-732, Dyomics-734, Dyomics-749, Dyomics-749P1, Dyomics-750, Dyomics-751, Dyomics-752, Dyomics-754, Dyomics-776, Dyomics-777, Dyomics-778, Dyomics-780, Dyomics-781, Dyomics-782, Dyomics-800, Dyomics-831, eFluor® 450, Eosin, FITC, Fluorescein, HiLyte™ Fluor 405, HiLyte™ Fluor 488, HiLyte™ Fluor 532, HiLyte™ Fluor 555, HiLyte™ Fluor 594, HiLyte™ Fluor 647, HiLyte™ Fluor 680, HiLyte™ Fluor 750, IRDye® 680LT, IRDye® 750, IRDye® 800CW, JOE, LightCycler® 640R, LightCycler® Red 610, LightCycler® Red 640, LightCycler® Red 670, LightCycler® Red 705, Lissamine Rhodamine B, Napthofluorescein, Oregon Green® 488, Oregon Green® 514, Pacific Blue™, Pacific Green™, Pacific Orange™, PET, PF350, PF405, PF415, PF488, PF505, PF532, PF546, PF555P, PF568, PF594, PF610, PF633P, PF647P, Quasar® 570, Quasar® 670, Quasar® 705, Rhodamine 123, Rhodamine 6G, Rhodamine B, Rhodamine Green, Rhodamine Green-X, Rhodamine Red, ROX, Seta™ 375, Seta™ 470, Seta™ 555, Seta™ 632, Seta™ 633, Seta™ 650, Seta™ 660, Seta™ 670, Seta™ 680, Seta™ 700, Seta™ 750, Seta™ 780, Seta™ APC-780, Seta™ PerCP-680, Seta™ R-PE-670, Seta™ 646, SeTau 380, SeTau 425, SeTau 647, SeTau 405, Square 635, Square 650, Square 660, Square 672, Square 680, Sulforhodamine 101, TAMRA, TET, Texas Red®, TMR, TRITC, Yakima Yellow™, Zenon®, Zy3, Zy5, Zy5.5, and Zy7.

### E. Luminescence

In some aspects, the application relates to polypeptide sequencing and/or identification based on one or more luminescence properties of a luminescent label. In some embodiments, a luminescent label is identified based on luminescence lifetime, luminescence intensity, brightness, absorption spectra, emission spectra, luminescence quantum yield, or a combination of two or more thereof. In some embodiments, a plurality of types of luminescent labels can be distinguished from each other based on different luminescence lifetimes, luminescence intensities, brightnesses, absorption spectra, emission spectra, luminescence quantum yields, or combinations of two or more thereof. Identifying may mean assigning the exact identity and/or quantity of one type of amino acid (e.g., a single type or a subset of types) associated with a luminescent label, and may also mean assigning an amino acid location in a polypeptide relative to other types of amino acids.

In some embodiments, luminescence is detected by exposing a luminescent label to a series of separate light pulses and evaluating the timing or other properties of each photon that is emitted from the label. In some embodiments, information for a plurality of photons emitted sequentially from a label is aggregated and evaluated to identify the label and thereby identify an associated type of amino acid. In some embodiments, a luminescence lifetime of a label is determined from a plurality of photons that are emitted sequentially from the label, and the luminescence lifetime can be used to identify the label. In some embodiments, a luminescence intensity of a label is determined from a plurality of photons that are emitted sequentially from the label, and the luminescence intensity can be used to identify the label. In some embodiments, a luminescence lifetime and luminescence intensity of a label is determined from a plurality of photons that are emitted sequentially from the label, and the luminescence lifetime and luminescence intensity can be used to identify the label.

In some aspects of the application, a single polypeptide molecule is exposed to a plurality of separate light pulses and a series of emitted photons are detected and analyzed. In some embodiments, the series of emitted photons provides information about the single polypeptide molecule that is present and that does not change in the reaction sample over the time of the experiment. However, in some embodiments, the series of emitted photons provides information about a series of different molecules that are present at different times in the reaction sample (e.g., as a reaction or process progresses). By way of example and not limitation, such information may be used to sequence and/or identify a polypeptide subjected to chemical or enzymatic degradation in accordance with the application.

In certain embodiments, a luminescent label absorbs one photon and emits one photon after a time duration. In some embodiments, the luminescence lifetime of a label can be determined or estimated by measuring the time duration. In some embodiments, the luminescence lifetime of a label can be determined or estimated by measuring a plurality of time durations for multiple pulse events and emission events. In some embodiments, the luminescence lifetime of a label can be differentiated amongst the luminescence lifetimes of a plurality of types of labels by measuring the time duration. In some embodiments, the luminescence lifetime of a label can be differentiated amongst the luminescence lifetimes of a plurality of types of labels by measuring a plurality of time durations for multiple pulse events and emission events. In certain embodiments, a label is identified or differentiated amongst a plurality of types of labels by determining or estimating the luminescence lifetime of the label. In certain embodiments, a label is identified or differentiated amongst a plurality of types of labels by differentiating the luminescence lifetime of the label amongst a plurality of the luminescence lifetimes of a plurality of types of labels.

Determination of a luminescence lifetime of a luminescent label can be performed using any suitable method (e.g., by measuring the lifetime using a suitable technique or by determining time-dependent characteristics of emission). In some embodiments, determining the luminescence lifetime of one label comprises determining the lifetime relative to another label. In some embodiments, determining the luminescence lifetime of a label comprises determining the lifetime relative to a reference. In some embodiments, determining the luminescence lifetime of a label comprises measuring the lifetime (e.g., fluorescence lifetime). In some embodiments, determining the luminescence lifetime of a label comprises determining one or more temporal characteristics that are indicative of lifetime. In some embodiments, the luminescence lifetime of a label can be determined based on a distribution of a plurality of emission events (e.g., 1, 2, 3, 4, 5, 6, 7, 8, 9, 10, 11, 12, 13, 14, 15, 16, 17, 18, 19, 20, 30, 40, 50, 60, 70, 80, 90, 100, or more emission events) occurring across one or more time-gated windows relative to an excitation pulse. For example, a luminescence lifetime of a label can be distinguished from a plurality of labels having different luminescence lifetimes based on the distribution of photon arrival times measured with respect to an excitation pulse.

It should be appreciated that a luminescence lifetime of a luminescent label is indicative of the timing of photons emitted after the label reaches an excited state and the label can be distinguished by information indicative of the timing of the photons. Some embodiments may include distinguishing a label from a plurality of labels based on the luminescence lifetime of the label by measuring times associated with photons emitted by the label. The distribution of times may provide an indication of the luminescence lifetime which may be determined from the distribution. In some embodiments, the label is distinguishable from the plurality of labels based on the distribution of times, such as by comparing the distribution of times to a reference distribution corresponding to a known label. In some embodiments, a value for the luminescence lifetime is determined from the distribution of times.

As used herein, in some embodiments, luminescence intensity refers to the number of emitted photons per unit time that are emitted by a luminescent label which is being excited by delivery of a pulsed excitation energy. In some embodiments, the luminescence intensity refers to the detected number of emitted photons per unit time that are emitted by a label which is being excited by delivery of a pulsed excitation energy, and are detected by a particular sensor or set of sensors.

As used herein, in some embodiments, brightness refers to a parameter that reports on the average emission intensity per luminescent label. Thus, in some embodiments, “emission intensity” may be used to generally refer to brightness of a composition comprising one or more labels. In some embodiments, brightness of a label is equal to the product of its quantum yield and extinction coefficient.

As used herein, in some embodiments, luminescence quantum yield refers to the fraction of excitation events at a given wavelength or within a given spectral range that lead to an emission event, and is typically less than 1. In some embodiments, the luminescence quantum yield of a luminescent label described herein is between 0 and about 0.001, between about 0.001 and about 0.01, between about 0.01 and about 0.1, between about 0.1 and about 0.5, between about 0.5 and 0.9, or between about 0.9 and 1. In some embodiments, a label is identified by determining or estimating the luminescence quantum yield.

As used herein, in some embodiments, an excitation energy is a pulse of light from a light source. In some embodiments, an excitation energy is in the visible spectrum. In some embodiments, an excitation energy is in the ultraviolet spectrum. In some embodiments, an excitation energy is in the infrared spectrum. In some embodiments, an excitation energy is at or near the absorption maximum of a luminescent label from which a plurality of emitted photons are to be detected. In certain embodiments, the excitation energy is between about 500 nm and about 700 nm (e.g., between about 500 nm and about 600 nm, between about 600 nm and about 700 nm, between about 500 nm and about 550 nm, between about 550 nm and about 600 nm, between about 600 nm and about 650 nm, or between about 650 nm and about 700 nm). In certain embodiments, an excitation energy may be monochromatic or confined to a spectral range. In some embodiments, a spectral range has a range of between about 0.1 nm and about 1 nm, between about 1 nm and about 2 nm, or between about 2 nm and about 5 nm. In some embodiments, a spectral range has a range of between about 5 nm and about 10 nm, between about 10 nm and about 50 nm, or between about 50 nm and about 100 nm.

### IV. Kits for Sample Preparation

In some aspects, the disclosure relates to kits for preparing a polypeptide sample (e.g., an enriched sample) for sequencing. A kit may be sufficient to prepare one or more polypeptide samples (e.g., enriched samples) for sequencing. In some embodiments, a kit is sufficient to prepare a single polypeptide sample. In other embodiments, a kit is sufficient to prepare, at least 2, at least 3, at least 4, at least 5, at least 6, at least 7, at least 8, at least 9, at least 10, at least 11, at least 12, at least 13, at least 14, at least 15, at least 20, at least 25, at least 30, at least 40, at least 50, at least 60, at least 70, at least 80, at least 90, or at least 100 polypeptide samples.

In some embodiments, a kit comprises an enrichment component comprising a a plurality of enrichment molecules, as described herein. See “Methods of Polypeptide Enrichment.” In some embodiments, a kit comprises a modifying agent, as described herein. See “Methods of Polypeptide Enrichment.” In some embodiments, a kit comprises an affinity reagent, as described herein. See “Polypeptide Sequencing Methodologies.” In some embodiments, a kit comprises a labeled peptidase, as described herein. See “Polypeptide Sequencing Methodologies”.

A kit may be specific for one or more organisms (e.g., one or more single-cellular and/or multicellular organisms). In some embodiments, a kit comprises components (e.g., enrichment molecules, modifying agents, or a combination thereof) that modify, bind to, are bound by, etc., polypeptides of one or more organisms. For example, in some embodiments, a kit comprises components that modify, bind to, are bound by, etc., one or more known polypeptides in the human proteome.

In some embodiments, a kit is specific for one or more disease or condition. For example, a kit may be an oncology kit, a cardiology kit, an inherited disease kit, a bacterial virulence factor kit, an antibiotic resistance kit, or a combination thereof.

An oncology kit may comprise enrichment molecules that bind to (or are bound by) ABL1, ABL2, ACSL3, ACVR2A, ADAMTS20, ADGRA2, ADGRB3, ADGRL3, AFF1, AFF3, AKAP9, AKT1, AKT2, AKT3, ALK, AMER1, APC, AR, ARID1A, ARID2, ARNT, ASXL1, ATF1, ATM, ATR, ATRX, AURKA, AURKB, AURKC, AXL, BAP1, BCL10, BCL11A, BCL11B, BCL2, BCL2L1, BCL2L2, BCL3, BCL6, BCL7A, BCL9, BCR, BIRC2, BIRC3, BIRC5, BLM, BLNK, BMPR1A, BRAF, BRCA1, BRCA2, BRD3, BRIP1, BTK, BUB1B, CACNA1D, CARD11, CASC5, CASP8, CBFA2T3, CBFB, CBL, CCND1, CCND2, CCNE1, CD79A, CD79B, CDCl73, CDH1, CDH11, CDH2, CDH20, CDH5, CDK12, CDK4, CDK6, CDK8, CDKN2A, CDKN2B, CDKN2C, CEBPA, CHEK1, CHEK2, CIC, CKS1B, CMPK1, COL1A1, CRBN, CREB1, CREBBP, CRKL, CRLF2, CRTC1, CSF1R, CSMD3, CTNNA1, CTNNB1, CYLD, CYP2C19, CYP2D6, DAXX, DCC, DDB2, DDIT3, DDR2, DEK, DICER1, DNMT3A, DPYD, DST, EGFR, EML4, EP300, EP400, EPHA3, EPHA7, EPHB1, EPHB4, EPHB6, ERBB2, ERBB3, ERBB4, ERCC1, ERCC2, ERCC3, ERCC4, ERCC5, ERG, ESR1, ETS1, ETV1, ETV4, EXT1, EXT2, EZH2, FANCA, FANCC, FANCD2, FANCF, FANCG, FAS, FBXW7, FCGR2B, FGFR1, FGFR2, FGFR3, FGFR4, FH, FLCN, FLI1, FLT1, FLT3, FLT4, FN1, FOXA1, FOXL2, FOXO1, FOXO3, FOXP1, FOXP4, FZR1, G6PD, GATA1, GATA2, GATA3, GDNF, GNA11, GNAQ, GNAS, GPC3, GRM8, GUCY1A2, HCAR1, HEY1, HIF1A, HIST1H3B, HLF, HMGA1, HNF1A, HOOK3, HOXA13, HOXD11, HRAS, HSP90AA1, HSP90AB1, ICK, IDH1, IDH2, IGF1R, IGF2, IGF2R, IKBKB, IKBKE, IKZF1, IL2, IL21R, IL6ST, IL7R, ING4, IRF4, IRS2, ITGA10, ITGA9, ITGB2, ITGB3, JAK1, JAK2, JAK3, JUN, KAT6A, KAT6B, KDM5C, KDM6A, KDR, KEAP1, KIAA1549, KIT, KLF6, KMT2A, KMT2C, KMT2D, KRAS, LAMP1, LCK, LIFR, LPP, LRP1B, LTF, LTK, MAF, MAFB, MAGEA1, MAGI1, MALT1, MAML2, MAP2K1, MAP2K2, MAP2K4, MAP3K7, MAPK1, MAPK8, MARK1, MARK4, MBD1, MCL1, MDM2, MDM4, MEN1, MET, MITF, MLH1, MLLT10, MLLT4, MLLT6, MMP2, MN1, MPL, MRE11A, MSH2, MSH6, MTCP1, MTOR, MTR, MTRR, MUC1, MUTYH, MYB, MYC, MYCL, MYCN, MYD88, MYH11, MYH9, NBN, NCOA1, NCOA2, NCOA4, NF1, NF2, NFE2L2, NFKB1, NFKB2, NIN, NKX2-1, NLRP1, NOTCH1, NOTCH2, NOTCH4, NPM1, NR4A3, NRAS, NSD1, NTRK1, NTRK3, NUMA1, NUP214, NUP98, NUTM2A, NUTM2B, OMD, P2RY8, PAK3, PALB2, PARP1, PAX3, PAX5, PAX7, PAX8, PBRM1, PBX1, PDE4DIP, PDGFB, PDGFRA, PDGFRB, PERI, PGAP3, PHOX2B, PIK3C2B, PIK3CA, PIK3CB, PIK3CD, PIK3CG, PIK3R1, PIK3R2, PIM1, PKHD1, PLAG1, PLCG1, PLEKHG5, PML, PMS1, PMS2, POT1, POU5F1, PPARG, PPP2R1A, PRDM1, PRKAR1A, PRKDC, PSIP1, PTCH1, PTEN, PTGS2, PTPN11, PTPRD, PTPRT, RAD50, RAF1, RALGDS, RAP1GDS1, RARA, RB1, RECQL4, REL, RET, RHOH, RNASEL, RNF2, RNF213, ROS1, RPS6KA2, RRM1, RUNX1, RUNX1T1, SAMD9, SBDS, SDHA, SDHB, SDHC, SDHD, SET, SETBP1, SETD2, SF3B1, SGK1, SH2D1A, SH3GL1, SMAD2, SMAD4, SMARCA4, SMARCB1, SMO, SMUG1, SOCS1, SOX11, SOX2, SRC, SSX1, SSX2, SSX4, STAT5B, STK11, STK36, SUFU, SYK, SYNE1, TAF1, TAF1L, TAL1, TBL1XR1, TBX22, TCF12, TCF3, TCF7L1, TCF7L2, TCL1A, TERT, TET1, TET2, TFE3, TGFBR2, TGM7, THBS1, TIMP3, TLR4, TLX1, TMPRSS2, TNFAIP3, TNFRSF14, TNK2, TOP1, TP53, TPR, TRIM24, TRIM33, TRIP11, TRRAP, TSC1, TSC2, TSHR, TTL, UBR5, UGT1A1, USP9X, VHL, WAS, WHSC1, WRN, WT1, XPA, XPC, XPO1, XRCC2, ZNF384, ZNF521, or any combination thereof.

A cardiology kit may comprise enrichment molecules that bind to (or are bound by) ABCC9, ABCG5, ABCG8, ACTA1, ACTA2, ACTC1, ACTN2, AKAP9, ALMS1, ANK2, ANKRD1, APOA4, APOA5, APOB, APOC2, APOE, BAG3, BRAF, CACNA1C, CACNA2D1, CACNB2, CALM1, CALR3, CASQ2, CAV3, CBL, CBS, CETP, COL3A1, COL5A1, COL5A2, COX15, CREB3L3, CRELD1, CRYAB, CSRP3, CTF1, DES, DMD, DNAJC19, DOLK, DPP6, DSC2, DSG2, DSP, DTNA, EFEMP2, ELN, EMD, EYA4, FBN1, FBN2, FHL1, FHL2, FKRP, FKTN, FXN, GAA, GATAD1, GCKR, GJA5, GLA, GPD1L, GPIHBP1, HADHA, HCN4, HFE, HRAS, HSPB8, ILK, JAG1, JPH2, JUP, KCNA5, KCND3, KCNE1, KCNE2, KCNE3, KCNH2, KCNJ2, KCNJ5, KCNJ8, KCNQ1, KLF10, KRAS, LAMA2, LAMA4, LAMP2, LDB3, LDLR, LDLRAP1, LMF1, LMNA, LPL, LTBP2, MAP2K1, MAP2K2, MIB1, MURC, MYBPC3, MYH11, MYH6, MYH7, MYL2, MYL3, MYLK, MYLK2, MYO6, MYOZ2, MYPN, NEXN, NKX2-5, NODAL, NOTCH1, NPPA, NRAS, PCSK9, PDLIM3, PKP2, PLN, PRDM16, PRKAG2, PRKAR1A, PTPN11, RAF1, RANGRF, RBM20, RYR1, RYR2, SALL4, SCN1B, SCN2B, SCN3B, SCN4B, SCN5A, SCO2, SDHA, SEPN1, SGCB, SGCD, SGCG, SHOC2, SLC25A4, SLC2A10, SMAD3, SMAD4, SNTA1, SOS1, SREBF2, TAZ, TBX20, TBX3, TBX5, TCAP, TGFB2, TGFB3, TGFBR1, TGFBR2, TMEM43, TMPO, TNNC1, TNNI3, TNNT2, TPM1, TRDN, TRIM63, TRPM4, TTN, TTR, TXNRD2, VCL, ZBTB17, ZHX3, and/or ZIC3.

An inherited disease kit may comprise enrichment molecules that bind to (or are bound by) ABCA4, ABCC9, ABCD1, ACADVL, ACTA2, ACTC1, ACTN2, ADA, AIPL1, AIRE, AKAP9, ALPL, AMT, ANK2, APC, APP, APTX, ARL6, ARSA, ASL, ASPA, ATL1, ATM, ATP2A2, ATP7A, ATP7B, ATXN1, ATXN2, ATXN7, BAG3, BCKDHA, BCKDHB, BEST1, BMPR1A, BTD, BTK, CA4, CACNA1C, CACNB2, CALR3, CAPN3, CASQ2, CAV3, CCDCl39, CCDC40, CDH23, CEP290, CERKL, CFTR, CHAT, CHD7, CHEK2, CHM, CHRNA1, CHRNB1, CHRND, CHRNE, CLCN1, CNGB1, COL11A1, COL11A2, COL1A1, COL1A2, COL2A1, COL3A1, COL4A1, COL4A5, COL5A1, COL5A2, COL7A1, COL9A1, CRB1, CRX, CTDP1, CTNS, CYP27A1, DBT, DCX, DES, DHCR7, DKC1, DLD, DMD, DNAH11, DNAH5, DNAH9, DNAI1, DNAI2, DNM2, DOK7, DSC2, DSG2, DSP, DYSF, ELN, EMD, ENG, EXT1, EYA1, EYS, F8, F9, FANCA, FANCC, FANCF, FANCG, FBN1, FBXO7, FGFR1, FGFR3, FMO3, FOXL2, FRG1, FRMD7, FSCN2, FXN, GAA, GALT, GATA4, GBA, GBE1, GCSH, GDF5, GJB2, GJB3, GJB6, GLA, GLDC, GNE, GNPTAB, GPC3, GPD1L, GPR143, GUCY2D, HBA2, HBB, HCN4, HEXA, HFE, HIBCH, HMBS, HR, IDS, IDUA, IKBKAP, IL2RG, IMPDH1, ITGB4, JAG1, JUP, KCNE1, KCNE2, KCNE3, KCNH2, KCNJ2, KCNQ1, KCNQ4, KIAA0196, KLHL7, KRAS, KRT14, KRT5, L1CAM, LAMB3, LAMP2, LDB3, LMNA, LRAT, LRRK2, MAPT, MC1R, MECP2, MED12, MEN1, MERTK, MFN2, MLH1, MMAA, MMAB, MMACHC, MPZ, MSH2, MTM1, MUT, MYBPC3, MYH11, MYH6, MYH7, MYL2, MYL3, MYLK, MYO7A, MYOZ2, NF1, NF2, NIPBL, NKX2-5, NME8, NPC1, NPC2, NR2E3, NRAS, NSD1, OCA2, OCRL, OTC, PABPN1, PAFAH1B1, PAH, PAX3, PAX6, PCDH15, PEX1, PEX10, PEX13, PEX14, PEX19, PEX26, PEX3, PEX5, PINK1, PKD1, PKD2, PKHD1, PKP2, PLEC, PLN, PLOD1, PMM2, PMP22, POLG, PPT1, PRCD, PRKAG2, PROM1, PRPF31, PRPF8, PRPH2, PSEN1, PSEN2, PTCH1, PTPN11, RAF1, RAG1, RAG2, RAI1, RAPSN, RB1, RDH12, RET, RHO, ROR2, RP9, RPE65, RPGR, RPGRIP1, RPL11, RPL35A, RPS10, RPS19, RPS24, RPS26, RPS6KA3, RPS7, RS1, RSPH4A, RSPH9, RYR1, RYR2, SALL4, SCN1B, SCN3B, SCN4B, SCN5A, SCN9A, SEMA4A, SERPINA1, SERPING1, SGCD, SH3BP2, SIX1, SIX5, SLC25A13, SLC25A4, SLC26A4, SMAD3, SMAD4, SNCA, SNRNP200, SNTA1, SOD1, SOS1, SOX9, SPATA7, SPG7, STARD3, TAF1, TAZ, TBX5, TCOF1, TGFBR1, TGFBR2, TMEM43, TNNC1, TNNI3, TNNT1, TNNT2, TNXB, TOPORS, TP53, TPM1, TSC1, TSC2, TTPA, TTR, TULP1, TWIST1, TYR, USH1C, USH2A, VCL, VHL, WAS, WRN, WT1, or any combination thereof.

A bacterial virulence factor kit may comprise enrichment molecules that bind to (or are bound by) <alpha>-C protein, <alpha>-hemolysin, <beta>-C protein, <beta>-haemolysin/cytolysin, <beta>-hemolysin, <delta>-hemolysin, <gamma>-hemolysin, <i>lsp</i>T2SS, AAFs, ACF, AI-2, ALO, AS, Ace, Acid phosphatase, Acinetobactin, Acm, AcrAB, ActA, AdeFGH efflux pump, Adhesive fimbriae, Adr1, Adr2, AdsA, Aerobactin, Aerolysin, Afa/Dr family, Agf, AhpC, Ail, AipA, Alginate, Alkaline protease, Allantion utilization, Ami, AnsP, Anthrax toxin, Antigen 85, ArgP, AslA, Asp14, AtxA, Aureolysin, Auto, Autolysin, BFP, BSH, BabA, BadA/Vomp, Bap, BapC, BfmRS, BimA, Biotin synthesis, BoNT, BoaA, BoaB, BopD, Brk, Bsa T3SS, Bs1A, BtpA/Btpl/TcpB, BtpB, BvgAS, BvrR-BvrS, C2 toxin, C3 toxin, C5a peptidase, C<beta>G, CAI-1, CAMP factor, CARDS toxin, CBPs, CDT, CHIPS, CNA, CNF-1, CNF<s>y</s>, CPAF, CPE, CT, CadF, CagA, Capsule, Capsule I, CbpA/PspC, CcmC, CdpA, CdtB, Chu, CiaB, CiaC, Cif, ClpC, ClpE, ClpP, Clumping factor, Colibactin, CsrA, Csu fimbriae, Cya, CytK, Cytadherence organelle, Cytolysin, DNase, DT, DevRS, DipA, Dispersin, Dnt, Dot/Icm, Dot/Icm T4SS, Dr adhesins, EAST1, ECP, EF-Tu, ESAT-6/CFP-10, ESX-1, ESX-3, ESX-5, Eap/Map, Ebp pili, EbpS, EcbA, Efa-1/LifA, EfaA, Ent, Enterobactin, Erp, Esp, EspA, EspB, EspC, EspD, EspF, EspG, EspH, EspP, EtpA, Exe T2SS, Exfoliative toxin, ExoA, ExoS, ExoT, ExoU, ExoY, F1 antigen, F1C fimbriae, FBPs, FHA, FadD33, FarAB, FbpA, FbpABC, FbsA, FbsB, FdeC, FeoAB, Fimbriae, Flagella, Flp type IV pili, FmvB, FnBPs, FrgA, FsaP, Fsr, FupA, Fur, GGT, GRAB, GadC, Gelatinase, GrvA, GspA, GtcA, HBL, HMW1/HMW2, HP-NAP, HSI-I, Haemagglutinating pili, Hap, HbhA, Heat-labile toxin (LT), Heat-stable toxin (ST), Hemolysin, Hgp, HhuA, Hia/Hsf, HitABC, HmbR, HopZ, Hpt, HpuAB, Hsp60, HspR, HspX, HxuABC, Hyaluronate lyase, Hyaluronic acid capsule, Hyaluronidase, Ibes, IcsA (VirG), IcsP (SopA), IdeR, IdeS, IgA1 protease, IleP, IlpA, InhA, InlA, InlB, InlC, InlF, InlJ, InlK, InlP, Intercellular adhesion proteins, Intimin, Invasin, Invasin B/Ifp, Invasin C/Ilp, Invasin D, IraAB, IroN, Isd, Isocitrate lyase, JlpA, K1 capsule, KatA, KatAB, KatG, LAM, LLO, LLS, LOS, LPS, Lap, LapB, LasA, LasB, Lateral flagella, Lbp, Legiobactin, Ler, LetA/S, Lewis antigen, LigA, LipF, Lipase, Lmb, LntA, LpeA, Lpf, LplA1, Lsp, Lymphostatin/LifA, M protein, MAM7, MARTX, MOMP, MSHA pili, MSHA type IV pili, Map, MgtBC, MgtC, Mig-14, Mig-5, Mip, MisL, MmaA4, MntABC, Mpl, MprAB, MsrAB, MtrCDE, Mycobactin, Myf/pH6 antigen, Neuraminidase, Nhe, Nitrate reductase, NleA/EspI, NleC, NleD, NspA, O-antigen, OapA, OatA, OipA, OmpA, OmpU, Opa, Opc, P fimbriae, P2 protein, P44/Msp2 family, P5 protein, P97/P102 paralog family, PDIM, PE/PE-PGRS, PEB1, PI-1, PI-2, PI-2a, PLC, PNAG, PVL, Paa, PanC/PanD, PavA, PavB, PbpG, PcaA, Pef, Per, Pertactin, Pet, PfbA, PgdA, PhoP, PhoPQ, Phospholipase A2, Phospholipase C, Phospholipase D, Pht, Pic, Pili, Pla, PlcA, PlcB, Pld, Pneumolysin, Polar flagella, Porin, PrfA, PrsA2, PsaA, PspA, Ptx, Purine biosynthesis, Pyochelin, Pyocyanin, Pyoverdine, Pyrimidine biosynthesis, Quorom sensing, Quorum sensing, Quorum-sensing, RatB, Rck, RcsAB, RecN, RelA, Rhamnolipid, Rhizoferrin, RicA, RickA, RipA, RmpA, RpoS, RtxA, Rvh T4SS, S fimbriae, SCIN, SDr, SE, SIC, SLO, SLS, SMase, SabA, Sal, Sat, Sbi, Scal, Sca2, Sca4, Scm, SgrA, ShET1, ShET2, ShdA, Shiga toxin, Shu, SigA, SigE, SigF, SigH, SinH, SodA, SodB, SodC, SodCI, SpA, SpaP, SpeB, Spes, SprE, Spy, Staphopain, Staphylocoagulase, Staphylokinase, StcE, Streptokinase, Stx, Surface lipoproteins, SvpA, T2SS, T3SS, T3SS1, T3SS2, T6SS, T6SS-1, T7SS, TCP, TCT, TDH, TRH, TSST-1, TTSS, TTSS(SPI-1 encode), TTSS(SPI-2 encode), Tap type IV pili, Tbp, TcdA, TcdB, TcfA, TcpC, TeNT, Tir, TlyC, ToxB, TraJ, Trw type IV secretion system, Tsh, Type 1 fimbriae, Type 3 fimbriae, Type I fimbriae, Type I pili, Type IV pili, Type IV secretion system, Type VII secretion system, Urease, V8 protease, VCC, VacA, Vi antigen, Vip, VirB type IV secretion system, VirB/VirD4 type IV secretion system, VpadF, WhiB3, YadA, YapC, YapE, YapJ, YapK, YapV, YaxAB, Ybt, Yersiniabactin, Ymt, Yst, Zot, alpha-clostripain, alpha-toxin (CpPLC), alpha-toxin (novyi), alpha-toxin (septicum), beta-toxin, beta2-toxin, enh loci, epsilon-toxin, fHbp, iota-toxin, kappa-toxin, mu-toxin, p60, pilus, pmiA, rOmpA/Sca0, rOmpB/Sca5, sialidase, theta-toxin/PFO, vWbp, xcp secretion system, or any combination thereof.

An antibiotic resistance kit may comprise enrichment molecules that bind to (or are bound by) AAC(1)-I, AAC(2′)-IIa, AAC(2′)-IIb, AAC(2′)-Ia, AAC(2′)-Ib, AAC(2′)-Ic, AAC(2′)-Id, AAC(2′)-Ie, AAC(3)-IIIa, AAC(3)-IIIb, AAC(3)-IIIc, AAC(3)-IIa, AAC(3)-IIb, AAC(3)-IIc, AAC(3)-IId, AAC(3)-IIe, AAC(3)-IV, AAC(3)-IXa, AAC(3)-Ia, AAC(3)-Ib, AAC(3)-Ib/AAC(6′)-Ib″, AAC(3)-Ic, AAC(3)-Id, AAC(3)-VIIIa, AAC(3)-VIIa, AAC(3)-VIa, AAC(3)-Xa, AAC(6′)-29a, AAC(6′)-29b, AAC(6′)-30/AAC(6′)-Ib′ fusion protein, AAC(6′)-31, AAC(6′)-32, AAC(6′)-33, AAC(6′)-34, AAC(6′)-I30, AAC(6′)-IIa, AAC(6′)-IIb, AAC(6′)-IIc, AAC(6′)-Ia, AAC(6′)-Iaa, AAC(6′)-Iad, AAC(6′)-Iae, AAC(6′)-Iaf, AAC(6′)-Iag, AAC(6′)-Iai, AAC(6′)-Iaj, AAC(6′)-Iak, AAC(6′)-Ian, AAC(6′)-Ib, AAC(6′)-Ib′, AAC(6′)-Ib-Hangzhou, AAC(6′)-Ib-SK, AAC(6′)-Ib-Suzhou, AAC(6′)-Ib-cr, AAC(6′)-Ib10, AAC(6′)-Ib11, AAC(6′)-Ib3, AAC(6′)-Ib4, AAC(6′)-Ib7, AAC(6′)-Ib8, AAC(6′)-Ib9, AAC(6′)-Ic, AAC(6′)-Ie-APH(2″)-Ia, AAC(6′)-If, AAC(6′)-Ig, AAC(6′)-Ih, AAC(6′)-Ii, AAC(6′)-Iid, AAC(6′)-Iih, AAC(6′)-Ij, AAC(6′)-Ik, AAC(6′)-Il, AAC(6′)-Im, AAC(6′)-Ip, AAC(6′)-Iq, AAC(6′)-Ir, AAC(6′)-Is, AAC(6′)-Isa, AAC(6′)-It, AAC(6′)-Iu, AAC(6′)-Iv, AAC(6′)-Iw, AAC(6′)-Ix, AAC(6′)-Iy, AAC(6′)-Iz, ACC-1, ACC-2, ACC-3, ACC-4, ACC-5, ACI-1, ACT-1, ACT-10, ACT-12, ACT-13, ACT-14, ACT-15, ACT-16, ACT-17, ACT-18, ACT-19, ACT-2, ACT-20, ACT-21, ACT-22, ACT-23, ACT-24, ACT-25, ACT-27, ACT-28, ACT-29, ACT-3, ACT-30, ACT-31, ACT-32, ACT-33, ACT-35, ACT-36, ACT-37, ACT-38, ACT-4, ACT-5, ACT-6, ACT-7, ACT-8, ACT-9, ADC-1, ADC-10, ADC-11, ADC-12, ADC-13, ADC-14, ADC-15, ADC-16, ADC-17, ADC-18, ADC-19, ADC-2, ADC-20, ADC-21, ADC-22, ADC-23, ADC-25, ADC-3, ADC-30, ADC-31, ADC-39, ADC-4, ADC-41, ADC-42, ADC-43, ADC-44, ADC-5, ADC-56, ADC-58, ADC-59, ADC-6, ADC-60, ADC-61, ADC-62, ADC-67, ADC-68, ADC-7, ADC-73, ADC-74, ADC-75, ADC-76, ADC-77, ADC-78, ADC-79, ADC-8, ADC-81, ADC-82, AER-1, AIM-1, ANT(2″)-Ia, ANT(3″)-IIa, ANT(3″)-IIb, ANT(3″)-IIc, ANT(3″)-Ii-AAC(6′)-IId fusion protein, ANT(4′)-IIa, ANT(4′)-IIb, ANT(4′)-Ia, ANT(4′)-Ib, ANT(6)-Ia, ANT(6)-Ib, ANT(9)-Ia, APH(2″)-IIIa, APH(2″)-IIa, APH(2″)-IVa, APH(2″)-Ie, APH(2″)-If, APH(2″)-Ig, APH(3″)-Ia, APH(3″)-Ib, APH(3″)-Ic, APH(3′)-IIIa, APH(3′)-IIa, APH(3′)-IIb, APH(3′)-IIc, APH(3′)-IVa, APH(3′)-IX, APH(3′)-Ia, APH(3′)-Ib, APH(3′)-VI, APH(3′)-VIIIa, APH(3′)-VIIIb, APH(3′)-VIIa, APH(3′)-VIa, APH(3′)-Va, APH(3′)-Vb, APH(3′)-Vc, APH(4)-Ia, APH(4)-Ib, APH(6)-Ia, APH(6)-Ib, APH(6)-Ic, APH(6)-Id, APH(7″)-Ia, APH(9)-Ia, APH(9)-Ib, AQU-1, AQU-2, AQU-3, ARL-1, ARL-2, ARL-3, ARL-4, ARL-5, ARL-6, AST-1, AZECL-25, Acinetobacter baumannii AbaF, Acinetobacter baumannii AbaQ, Acinetobacter baumannii AbuO, Acinetobacter baumannii AmvA, Acinetobacter baumannii OprD conferring resistance to imipenem, Acinetobacter baumannii ampC beta-lactamase, Acinetobacter baumannii gyrA conferring resistance to fluoroquinolones, Acinetobacter baumannii parC conferring resistance to fluoroquinolone, AcrE, AcrF, AcrS, Agrobacterium fabrum chloramphenicol acetyltransferase, ArmR, AxyX, AxyY, AxyZ, BAT-1, BCL-1, BEL-1, BEL-2, BEL-3, BES-1, BIC-1, BIL-1, BJP-1, BKC-1, BPU-1, BRO-1, BRO-2, BRP(MBL), BUT-1, Bacillus clausii chloramphenicol acetyltransferase, Bacillus pumilus cat86, Bacillus subtilis mprF, Bacillus subtilis pgsA with mutation conferring resistance to daptomycin, BahA, Bartonella bacilliformis gyrA conferring resistance to fluoroquinolones, Bartonella bacilliformis gyrB conferring resistance to aminocoumarin, BcI, BcII, Bifidobacterium adolescentis rpoB mutants conferring resistance to rifampicin, Bifidobacterium ileS conferring resistance to mupirocin, Bla1, Bla2, Borreliella burgdorferi 16S rRNA mutation conferring resistance to gentamicin, Borreliella burgdorferi 16S rRNA mutation conferring resistance to kanamycin, Borreliella burgdorferi 16S rRNA mutation conferring resistance to spectinomycin, Borreliella burgdorferi murA with mutation conferring resistance to fosfomycin, Brachyspira hyodysenteriae 23S rRNA with mutation conferring resistance to tylosin, Brucella suis mprF, Burkholderia pseudomallei Omp38, CAM-1, CARB-1, CARB-10, CARB-12, CARB-14, CARB-16, CARB-17, CARB-18, CARB-19, CARB-2, CARB-20, CARB-21, CARB-22, CARB-23, CARB-3, CARB-4, CARB-5, CARB-6, CARB-7, CARB-8, CARB-9, CAU-1, CBP-1, CFE-1, CFE-2, CGA-1, CGB-1, CIA-1, CIA-2, CIA-3, CIA-4, CKO-1, CME-1, CMH-1, CMY-1, CMY-10, CMY-100, CMY-101, CMY-102, CMY-103, CMY-104, CMY-105, CMY-106, CMY-108, CMY-11, CMY-110, CMY-111, CMY-112, CMY-113, CMY-114, CMY-115, CMY-116, CMY-117, CMY-118, CMY-119, CMY-12, CMY-13, CMY-131, CMY-132, CMY-133, CMY-135, CMY-14, CMY-15, CMY-16, CMY-17, CMY-18, CMY-19, CMY-2, CMY-20, CMY-21, CMY-22, CMY-23, CMY-24, CMY-25, CMY-26, CMY-27, CMY-28, CMY-29, CMY-30, CMY-31, CMY-32, CMY-33, CMY-34, CMY-35, CMY-36, CMY-37, CMY-38, CMY-39, CMY-4, CMY-40, CMY-41, CMY-42, CMY-43, CMY-44, CMY-45, CMY-46, CMY-47, CMY-48, CMY-49, CMY-5, CMY-50, CMY-51, CMY-53, CMY-54, CMY-55, CMY-56, CMY-57, CMY-58, CMY-59, CMY-6, CMY-60, CMY-61, CMY-62, CMY-63, CMY-64, CMY-65, CMY-66, CMY-67, CMY-68, CMY-69, CMY-7, CMY-70, CMY-71, CMY-72, CMY-73, CMY-74, CMY-75, CMY-76, CMY-77, CMY-78, CMY-79, CMY-8, CMY-80, CMY-81, CMY-82, CMY-83, CMY-84, CMY-85, CMY-86, CMY-87, CMY-9, CMY-90, CMY-93, CMY-94, CMY-95, CMY-98, CMY-99, CPS-1, CRP, CTX-M-1, CTX-M-10, CTX-M-100, CTX-M-101, CTX-M-102, CTX-M-103, CTX-M-104, CTX-M-105, CTX-M-106, CTX-M-107, CTX-M-108, CTX-M-109, CTX-M-11, CTX-M-110, CTX-M-111, CTX-M-112, CTX-M-113, CTX-M-114, CTX-M-115, CTX-M-116, CTX-M-117, CTX-M-12, CTX-M-121, CTX-M-122, CTX-M-123, CTX-M-124, CTX-M-125, CTX-M-126, CTX-M-129, CTX-M-13, CTX-M-130, CTX-M-131, CTX-M-132, CTX-M-134, CTX-M-136, CTX-M-137, CTX-M-139, CTX-M-14, CTX-M-141, CTX-M-142, CTX-M-144, CTX-M-147, CTX-M-148, CTX-M-15, CTX-M-151, CTX-M-152, CTX-M-155, CTX-M-156, CTX-M-157, CTX-M-158, CTX-M-159, CTX-M-16, CTX-M-160, CTX-M-17, CTX-M-19, CTX-M-2, CTX-M-20, CTX-M-21, CTX-M-22, CTX-M-23, CTX-M-24, CTX-M-25, CTX-M-26, CTX-M-27, CTX-M-28, CTX-M-29, CTX-M-3, CTX-M-30, CTX-M-31, CTX-M-32, CTX-M-33, CTX-M-34, CTX-M-35, CTX-M-36, CTX-M-37, CTX-M-38, CTX-M-39, CTX-M-4, CTX-M-40, CTX-M-41, CTX-M-42, CTX-M-43, CTX-M-44, CTX-M-45, CTX-M-46, CTX-M-47, CTX-M-48, CTX-M-49, CTX-M-5, CTX-M-50, CTX-M-51, CTX-M-52, CTX-M-53, CTX-M-54, CTX-M-55, CTX-M-56, CTX-M-58, CTX-M-59, CTX-M-6, CTX-M-60, CTX-M-61, CTX-M-62, CTX-M-63, CTX-M-64, CTX-M-65, CTX-M-66, CTX-M-67, CTX-M-68, CTX-M-69, CTX-M-7, CTX-M-71, CTX-M-72, CTX-M-74, CTX-M-75, CTX-M-76, CTX-M-77, CTX-M-78, CTX-M-79, CTX-M-8, CTX-M-80, CTX-M-81, CTX-M-82, CTX-M-83, CTX-M-84, CTX-M-85, CTX-M-86, CTX-M-87, CTX-M-88, CTX-M-89, CTX-M-9, CTX-M-90, CTX-M-91, CTX-M-92, CTX-M-93, CTX-M-94, CTX-M-95, CTX-M-96, CTX-M-98, CTX-M-99, Campylobacter coli chloramphenicol acetyltransferase, Campylobacter jejuni 23S rRNA with mutation conferring resistance to erythromycin, Campylobacter jejuni gyrA conferring resistance to fluoroquinolones, Capnocytophaga gingivalis gyrA conferring resistance to fluoroquinolones, CatU, CblA-1, CcrA, CepS, CfxA, CfxA2, CfxA3, CfxA4, CfxA5, CfxA6, Chlamydia trachomatis 23S rRNA with mutation conferring resistance to macrolide antibiotics, Chlamydia trachomatis intrinsic murA conferring resistance to fosfomycin, Chlamydomonas reinhardtii 16S rRNA (rrnS) mutation conferring resistance to streptomycin, Chlamydomonas reinhardtii 23S rRNA with mutation conferring resistance to erythromycin, Chlamydophila psittaci 16S rRNA mutation conferring resistance to spectinomycin, Chryseobacterium meningosepticum BlaB, Clostridioides difficile 23S rRNA with mutation conferring resistance to erythromycin and clindamycin, Clostridioides difficile EF-Tu mutants conferring resistance to elfamycin, Clostridioides difficile gyrA conferring resistance to fluoroquinolones, Clostridioides difficile gyrB conferring resistance to fluoroquinolone, Clostridioides difficile murG with mutation conferring resistance to vancomycin, Clostridioides difficile rpoB with mutation conferring resistance to rifampicin, Clostridioides difficile rpoC with mutation conferring resistance to vancomycin, Clostridium butyricum catB, Clostridium perfringens mprF, Corynebacterium striatum tetA, CrpP, Cutibacterium acnes 16S rRNA mutation conferring resistance to tetracycline, Cutibacterium acnes gyrA conferring resistance to fluoroquinolones, D-Ala-D-Ala ligase, DES-1, DHA-1, DHA-10, DHA-12, DHA-13, DHA-14, DHA-15, DHA-16, DHA-17, DHA-18, DHA-19, DHA-2, DHA-20, DHA-21, DHA-22, DHA-3, DHA-5, DHA-6, DHA-7, DHA-9, DIM-1, DnaA, EBR-1 beta-lactamase, EBR-2, ERP-1, ESP-1, EXO-1, EdeQ, Enterobacter cloacae acrA, Enterobacter cloacae rob, Enterococcus faecalis YvlB with mutation conferring daptomycin resistance, Enterococcus faecalis YybT with mutation conferring daptomycin resistance, Enterococcus faecalis chloramphenicol acetyltransferase, Enterococcus faecalis cls with mutation conferring resistance to daptomycin, Enterococcus faecalis drmA with mutation conferring daptomycin resistance, Enterococcus faecalis gdpD with mutation conferring daptomycin resistance, Enterococcus faecalis gshF with mutation conferring daptomycin resistance, Enterococcus faecalis liaF mutant conferring daptomycin resistance, Enterococcus faecalis liaR mutant conferring daptomycin resistance, Enterococcus faecalis liaS mutant conferring daptomycin resistance, Enterococcus faecium EF-Tu mutants conferring resistance to GE2270A, Enterococcus faecium chloramphenicol acetyltransferase, Enterococcus faecium cls conferring resistance to daptomycin, Enterococcus faecium liaF mutant conferring daptomycin resistance, Enterococcus faecium liaR mutant conferring daptomycin resistance, Enterococcus faecium liaS mutant conferring daptomycin resistance, EreA, EreA2, EreB, EreD, Erm(30), Erm(31), Erm(33), Erm(34), Erm(35), Erm(36), Erm(37), Erm(38), Erm(39), Erm(41), Erm(42), Erm(43), Erm(44)v, Erm(47), Erm(48), Erm(49), Erm(K), Erm(O)-lrm, ErmA, ErmB, ErmC, ErmD, ErmE, ErmF, ErmG, ErmH, ErmN, ErmO-srmA, ErmQ, ErmR, ErmS, ErmT, ErmU, ErmV, ErmW, ErmX, ErmY, Escherichia coli 16S rRNA (rrnB) mutation conferring resistance to spectinomycin, Escherichia coli 16S rRNA (rrnB) mutation conferring resistance to streptomycin, Escherichia coli 16S rRNA (rrnB) mutation conferring resistance to tetracycline, Escherichia coli 16S rRNA (rrsB) mutation conferring resistance to G418, Escherichia coli 16S rRNA (rrsB) mutation conferring resistance to gentamicin C, Escherichia coli 16S rRNA (rrsB) mutation conferring resistance to kanamycin A, Escherichia coli 16S rRNA (rrsB) mutation conferring resistance to neomycin, Escherichia coli 16S rRNA (rrsB) mutation conferring resistance to paromomycin, Escherichia coli 16S rRNA (rrsB) mutation conferring resistance to spectinomycin, Escherichia coli 16S rRNA (rrsB) mutation conferring resistance to streptomycin, Escherichia coli 16S rRNA (rrsB) mutation conferring resistance to tetracycline, Escherichia coli 16S rRNA (rrsB) mutation conferring resistance to tobramycin, Escherichia coli 16S rRNA (rrsC) mutation conferring resistance to kasugamicin, Escherichia coli 16S rRNA (rrsH) mutation conferring resistance to spectinomycin, Escherichia coli 16S rRNA mutation conferring resistance to edeine, Escherichia coli 23S rRNA with mutation conferring resistance to chloramphenicol, Escherichia coli 23S rRNA with mutation conferring resistance to clarithromycin, Escherichia coli 23S rRNA with mutation conferring resistance to clindamycin, Escherichia coli 23S rRNA with mutation conferring resistance to erythromycin and telithromycin, Escherichia coli 23S rRNA with mutation conferring resistance to oxazolidinone antibiotics, Escherichia coli CpxR, Escherichia coli CyaA with mutation conferring resistance to fosfomycin, Escherichia coli EF-Tu mutants conferring resistance to Enacyloxin IIa, Escherichia coli EF-Tu mutants conferring resistance to Pulvomycin, Escherichia coli EF-Tu mutants conferring resistance to kirromycin, Escherichia coli GlpT with mutation conferring resistance to fosfomycin, Escherichia coli LamB, Escherichia coli PtsI with mutation conferring resistance to fosfomycin, Escherichia coli UhpA with mutation conferring resistance to fosfomycin, Escherichia coli UhpT with mutation conferring resistance to fosfomycin, Escherichia coli acrA, Escherichia coli acrR with mutation conferring multidrug antibiotic resistance, Escherichia coli ampC beta-lactamase, Escherichia coli ampC1 beta-lactamase, Escherichia coli ampH beta-lactamase, Escherichia coli emrE, Escherichia coli fabG mutations conferring resistance to triclosan, Escherichia coli fabI mutations conferring resistance to isoniazid and triclosan, Escherichia coli folP with mutation conferring resistance to sulfonamides, Escherichia coli gyrA conferring resistance to fluoroquinolones, Escherichia coli gyrA with mutation conferring resistance to triclosan, Escherichia coli gyrB conferring resistance to aminocoumarin, Escherichia coli marR mutant conferring antibiotic resistance, Escherichia coli mdfA, Escherichia coli mipA, Escherichia coli murA with mutation conferring resistance to fosfomycin, Escherichia coli nfsA mutations conferring resistance to nitrofurantoin, Escherichia coli nfsB with mutation conferring resistance to nitrofurantoin, Escherichia coli ompF with mutation conferring resistance to beta-lactam antibiotics, Escherichia coli parC conferring resistance to fluoroquinolone, Escherichia coli parE conferring resistance to fluoroquinolones, Escherichia coli rob, Escherichia coli rpoB mutants conferring resistance to rifampicin, Escherichia coli soxR with mutation conferring antibiotic resistance, Escherichia coli soxS with mutation conferring antibiotic resistance, FAR-1, FEZ-1, FIM-1, FONA-1, FONA-2, FONA-3, FONA-4, FONA-5, FONA-6, FOX-1, FOX-10, FOX-2, FOX-3, FOX-4, FOX-5, FOX-7, FOX-8, FOX-9, FPH-1, FRI-1, FRI-2, FRI-3, FTU-1, FomA, FomB, FosA, FosA2, FosA3, FosA4, FosA5, FosA6, FosA7, FosB, FosB1, FosB3, FosB4, FosB5, FosB6, FosC, FosC2, FosD, FosK, FosX, FusF, GES-1, GES-10, GES-11, GES-12, GES-13, GES-14, GES-15, GES-16, GES-17, GES-18, GES-19, GES-2, GES-20, GES-21, GES-22, GES-23, GES-24, GES-26, GES-3, GES-4, GES-5, GES-6, GES-7, GES-8, GES-9, GIM-1, GIM-2, GOB-1, GOB-10, GOB-11, GOB-12, GOB-13, GOB-14, GOB-15, GOB-16, GOB-18, GOB-2, GOB-3, GOB-4, GOB-5, GOB-6, GOB-7, GOB-8, GOB-9, H-NS, HERA-1, HERA-2, HERA-3, HMB-1, Haemophilus influenzae PBP3 conferring resistance to beta-lactam antibiotics, Haemophilus parainfluenzae gyrA conferring resistance to fluoroquinolones, Haemophilus parainfluenzae parC conferring resistance to fluoroquinolones, Halobacterium halobium 23S rRNA mutation conferring resistance to chloramphenicol, Halobacterium salinarum 16S rRNA mutation conferring resistance to pactamycin, Helicobacter pylori 16S rRNA mutation conferring resistance to tetracycline, Helicobacter pylori 23S rRNA with mutation conferring resistance to clarithromycin, ICR-Mc, ICR-Mo, IMI-1, IMI-2, IMI-3, IMI-4, IMI-7, IMP-1, IMP-10, IMP-11, IMP-12, IMP-13, IMP-14, IMP-15, IMP-16, IMP-18, IMP-19, IMP-2, IMP-20, IMP-21, IMP-22, IMP-24, IMP-25, IMP-26, IMP-27, IMP-28, IMP-29, IMP-3, IMP-30, IMP-31, IMP-32, IMP-33, IMP-34, IMP-35, IMP-37, IMP-38, IMP-4, IMP-40, IMP-41, IMP-42, IMP-43, IMP-44, IMP-45, IMP-48, IMP-5, IMP-51, IMP-55, IMP-56, IMP-6, IMP-7, IMP-8, IMP-9, IND-1, IND-10, IND-11, IND-12, IND-14, IND-15, IND-2, IND-2a, IND-3, IND-4, IND-5, IND-6, IND-7, IND-8, IND-9, JOHN-1, KHM-1, KPC-10, KPC-11, KPC-12, KPC-13, KPC-14, KPC-15, KPC-16, KPC-17, KPC-19, KPC-2, KPC-22, KPC-24, KPC-3, KPC-4, KPC-5, KPC-6, KPC-7, KPC-8, KPC-9, Klebsiella aerogenes Omp36, Klebsiella aerogenes acrR with mutation conferring multidrug antibiotic resistance, Klebsiella mutant PhoP conferring antibiotic resistance to colistin, Klebsiella pneumoniae KpnE, Klebsiella pneumoniae KpnF, Klebsiella pneumoniae KpnG, Klebsiella pneumoniae KpnH, Klebsiella pneumoniae OmpK35, Klebsiella pneumoniae OmpK36, Klebsiella pneumoniae OmpK37, Klebsiella pneumoniae acrA, Klebsiella pneumoniae acrR with mutation conferring multidrug antibiotic resistance, Klebsiella pneumoniae ramR mutants, L1 beta-lactamase, LAT-1, LCR-1, LEN-1, LEN-10, LEN-11, LEN-12, LEN-13, LEN-14, LEN-15, LEN-16, LEN-18, LEN-19, LEN-2, LEN-20, LEN-21, LEN-22, LEN-23, LEN-24, LEN-26, LEN-3, LEN-4, LEN-5, LEN-6, LEN-7, LEN-8, LEN-9, LRA-1, LRA-10, LRA-12, LRA-13, LRA-17, LRA-18, LRA-19, LRA-2, LRA-3, LRA-5, LRA-7, LRA-8, LRA-9, Lactobacillus reuteri cat-TC, Laribacter hongkongensis ampC beta-lactamase, Listeria monocytogenes mprF, LlmA 23S ribosomal RNA methyltransferase, LnuP, LpeA, LpeB, LpxA, LpxC, LpxD, MCR-1.1, MCR-1.10, MCR-1.11, MCR-1.12, MCR-1.13, MCR-1.2, MCR-1.3, MCR-1.4, MCR-1.5, MCR-1.6, MCR-1.7, MCR-1.8, MCR-1.9, MCR-2.1, MCR-2.2, MCR-3.1, MCR-3.10, MCR-3.11, MCR-3.12, MCR-3.2, MCR-3.3, MCR-3.4, MCR-3.5, MCR-3.6, MCR-3.7, MCR-3.8, MCR-3.9, MCR-4.1, MCR-4.2, MCR-4.3, MCR-4.4, MCR-4.5, MCR-5.1, MCR-5.2, MCR-6.1, MCR-7.1, MCR-8.1, MCR-9.1, MR-1, MIR-10, MIR-11, MIR-12, MIR-13, MIR-14, MIR-15, MIR-16, MIR-17, MIR-2, MIR-3, MIR-4, MIR-5, MIR-6, MIR-8, MIR-9, MOX-1, MOX-2, MOX-3, MOX-4, MOX-5, MOX-6, MOX-7, MOX-8, MOX-9, MSI-1, MSI-OXA, MUS-1, MUS-2, MdtK, Mef(En2), MexA, MexB, MexC, MexD, MexE, MexF, MexG, MexH, Mexl, MexJ, MexK, MexL, MexR, MexS, MexT, MexV, MexW, MexZ, Moraxella catarrhalis 23S rRNA with mutation conferring resistance to macrolide antibiotics, Moraxella catarrhalis M35, Morganella morganii gyrB conferring resistance to fluoroquinolone, MuxA, MuxB, MuxC, MvaT, Mycobacterium avium 23S rRNA with mutation conferring resistance to clarithromycin, Mycobacterium intracellulare 23S rRNA with mutation conferring resistance to azithromycin, Mycobacterium intracellulare 23S rRNA with mutation conferring resistance to clarithromycin, Mycobacterium kansasii 23S rRNA with mutation conferring resistance to clarithromycin, Mycobacterium leprae folP with mutation conferring resistance to dapsone, Mycobacterium leprae gyrB conferring resistance to fluoroquinolone, Mycobacterium leprae rpoB mutations conferring resistance to rifampicin, Mycobacterium tuberculosis 16S rRNA mutation conferring resistance to amikacin, Mycobacterium tuberculosis 16S rRNA mutation conferring resistance to kanamycin, Mycobacterium tuberculosis 16S rRNA mutation conferring resistance to streptomycin, Mycobacterium tuberculosis 16S rRNA mutation conferring resistance to viomycin, Mycobacterium tuberculosis embA mutant conferring resistance to ethambutol, Mycobacterium tuberculosis embB with mutation conferring resistance to ethambutol, Mycobacterium tuberculosis embB with mutation conferring resistance to rifampicin, Mycobacterium tuberculosis embR mutant conferring resistance to ethambutol, Mycobacterium tuberculosis ethA with mutation conferring resistance to ethionamide, Mycobacterium tuberculosis folC with mutation conferring resistance to para-aminosalicylic acid, Mycobacterium tuberculosis gidB mutation conferring resistance to streptomycin, Mycobacterium tuberculosis gyrA conferring resistance to fluoroquinolones, Mycobacterium tuberculosis gyrB mutant conferring resistance to fluoroquinolone, Mycobacterium tuberculosis inhA mutations conferring resistance to isoniazid, Mycobacterium tuberculosis iniA mutant conferring resistance to Ethambutol, Mycobacterium tuberculosis iniB with mutation conferring resistance to ethambutol, Mycobacterium tuberculosis iniC mutant conferring resistance to ethambutol, Mycobacterium tuberculosis intrinsic murA conferring resistance to fosfomycin, Mycobacterium tuberculosis kasA mutant conferring resistance to isoniazid, Mycobacterium tuberculosis katG mutations conferring resistance to isoniazid, Mycobacterium tuberculosis mutant embC conferring resistance to ethambutol, Mycobacterium tuberculosis ndh with mutation conferring resistance to isoniazid, Mycobacterium tuberculosis pncA mutations conferring resistance to pyrazinamide, Mycobacterium tuberculosis ribD with mutation conferring resistance to para-aminosalicylic acid, Mycobacterium tuberculosis rpoB mutants conferring resistance to rifampicin, Mycobacterium tuberculosis rpsA mutations conferring resistance to Pyrazinamide, Mycobacterium tuberculosis rpsL mutations conferring resistance to Streptomycin, Mycobacterium tuberculosis thyA with mutation conferring resistance to para-aminosalicylic acid, Mycobacterium tuberculosis tlyA mutations conferring resistance to aminoglycosides, Mycobacterium tuberculosis variant bovis embB with mutation conferring resistance to ethambutol, Mycobacterium tuberculosis variant bovis ndh with mutation conferring resistance to isoniazid, Mycobacteroides abscessus 16S rRNA mutation conferring resistance to amikacin, Mycobacteroides abscessus 16S rRNA mutation conferring resistance to gentamicin, Mycobacteroides abscessus 16S rRNA mutation conferring resistance to kanamycin, Mycobacteroides abscessus 16S rRNA mutation conferring resistance to neomycin, Mycobacteroides abscessus 16S rRNA mutation conferring resistance to tobramycin, Mycobacteroides abscessus 23S rRNA with mutation conferring resistance to clarithromycin, Mycobacteroides chelonae 16S rRNA mutation conferring resistance to amikacin, Mycobacteroides chelonae 16S rRNA mutation conferring resistance to gentamicin C, Mycobacteroides chelonae 16S rRNA mutation conferring resistance to kanamycin A, Mycobacteroides chelonae 16S rRNA mutation conferring resistance to neomycin, Mycobacteroides chelonae 16S rRNA mutation conferring resistance to tobramycin, Mycobacteroides chelonae 23S rRNA with mutation conferring resistance to clarithromycin, Mycobaterium leprae gyrA conferring resistance to fluoroquinolones, Mycolicibacterium smegmatis 16S rRNA (rrsA) mutation conferring resistance to hygromycin B, Mycolicibacterium smegmatis 16S rRNA (rrsA) mutation conferring resistance to kanamycin A, Mycolicibacterium smegmatis 16S rRNA (rrsA) mutation conferring resistance to neomycin, Mycolicibacterium smegmatis 16S rRNA (rrsA) mutation conferring resistance to viomycin, Mycolicibacterium smegmatis 16S rRNA (rrsB) mutation conferring resistance to hygromycin B, Mycolicibacterium smegmatis 16S rRNA (rrsB) mutation conferring resistance to kanamycin A, Mycolicibacterium smegmatis 16S rRNA (rrsB) mutation conferring resistance to neomycin, Mycolicibacterium smegmatis 16S rRNA (rrsB) mutation conferring resistance to streptomycin, Mycolicibacterium smegmatis 16S rRNA (rrsB) mutation conferring resistance to viomycin, Mycolicibacterium smegmatis 23S rRNA with mutation conferring resistance to clarithromycin, Mycolicibacterium smegmatis ndh with mutation conferring resistance to isoniazid, Mycoplasma fermentans 23S rRNA with mutation conferring resistance to macrolide antibiotics, Mycoplasma gallisepticum 23S rRNA mutation conferring resistance to pleuromutilin antibiotics, Mycoplasma genitalium 23S rRNA mutations confers resistance to fluoroquinolone and macrolide antibiotics, Mycoplasma genitalium gyrA mutation confers resistance to fluoroquinolones, Mycoplasma genitalium parC mutations confers resistance to Moxifloxacin, Mycoplasma hominis 23S rRNA with mutation conferring resistance to macrolide antibiotics, Mycoplasma hominis parC conferring resistance to fluoroquinolone, Mycoplasma pneumoniae 23S rRNA mutation conferring resistance to erythromycin, NDM-1, NDM-10, NDM-11, NDM-12, NDM-13, NDM-14, NDM-17, NDM-2, NDM-3, NDM-4, NDM-5, NDM-6, NDM-7, NDM-8, NDM-9, NPS-1, Neisseria gonorrhoeae 16S rRNA mutation conferring resistance to spectinomycin, Neisseria gonorrhoeae gyrA conferring resistance to fluoroquinolones, Neisseria gonorrhoeae parC conferring resistance to fluoroquinolone, Neisseria gonorrhoeae porin PIB (por), Neisseria meningitidis 16S rRNA mutation conferring resistance to spectinomycin, Neisseria meningititis PBP2 conferring resistance to beta-lactam, NmcA, NmcR, OCH-1, OCH-2, OCH-3, OCH-4, OCH-5, OCH-6, OCH-7, OCH-8, OKP-A-1, OKP-A-10, OKP-A-11, OKP-A-12, OKP-A-13, OKP-A-14, OKP-A-15, OKP-A-16, OKP-A-2, OKP-A-3, OKP-A-4, OKP-A-5, OKP-A-6, OKP-A-7, OKP-A-8, OKP-A-9, OKP-B-1, OKP-B-10, OKP-B-11, OKP-B-12, OKP-B-13, OKP-B-17, OKP-B-18, OKP-B-19, OKP-B-2, OKP-B-20, OKP-B-3, OKP-B-4, OKP-B-5, OKP-B-6, OKP-B-7, OKP-B-8, OKP-B-9, OXA-1, OXA-10, OXA-100, OXA-101, OXA-103, OXA-104, OXA-106, OXA-107, OXA-108, OXA-109, OXA-11, OXA-110, OXA-111, OXA-112, OXA-113, OXA-114a, OXA-115, OXA-116, OXA-117, OXA-118, OXA-119, OXA-12, OXA-120, OXA-121, OXA-122, OXA-123, OXA-124, OXA-125, OXA-126, OXA-127, OXA-128, OXA-129, OXA-13, OXA-130, OXA-131, OXA-132, OXA-133, OXA-134, OXA-135, OXA-136, OXA-137, OXA-138, OXA-139, OXA-14, OXA-140, OXA-141, OXA-142, OXA-143, OXA-144, OXA-145, OXA-146, OXA-147, OXA-148, OXA-149, OXA-15, OXA-150, OXA-151, OXA-152, OXA-153, OXA-154, OXA-155, OXA-156, OXA-157, OXA-158, OXA-16, OXA-160, OXA-161, OXA-162, OXA-163, OXA-164, OXA-165, OXA-166, OXA-167, OXA-168, OXA-169, OXA-17, OXA-170, OXA-171, OXA-172, OXA-173, OXA-174, OXA-175, OXA-176, OXA-177, OXA-178, OXA-179, OXA-18, OXA-180, OXA-181, OXA-182, OXA-183, OXA-184, OXA-185, OXA-19, OXA-192, OXA-194, OXA-195, OXA-196, OXA-197, OXA-198, OXA-199, OXA-2, OXA-20, OXA-200, OXA-201, OXA-202, OXA-203, OXA-204, OXA-205, OXA-206, OXA-207, OXA-208, OXA-209, OXA-21, OXA-210, OXA-211, OXA-212, OXA-213, OXA-214, OXA-215, OXA-216, OXA-217, OXA-219, OXA-22, OXA-223, OXA-224, OXA-225, OXA-226, OXA-228, OXA-229, OXA-23, OXA-230, OXA-231, OXA-232, OXA-233, OXA-234, OXA-235, OXA-236, OXA-237, OXA-239, OXA-24, OXA-240, OXA-241, OXA-242, OXA-243, OXA-244, OXA-245, OXA-246, OXA-247, OXA-248, OXA-249, OXA-25, OXA-250, OXA-251, OXA-252, OXA-253, OXA-254, OXA-255, OXA-256, OXA-257, OXA-258, OXA-259, OXA-26, OXA-260, OXA-261, OXA-262, OXA-263, OXA-264, OXA-265, OXA-266, OXA-267, OXA-268, OXA-269, OXA-27, OXA-270, OXA-271, OXA-272, OXA-273, OXA-274, OXA-275, OXA-276, OXA-277, OXA-278, OXA-279, OXA-28, OXA-280, OXA-281, OXA-282, OXA-283, OXA-284, OXA-285, OXA-286, OXA-287, OXA-288, OXA-29, OXA-291, OXA-292, OXA-293, OXA-294, OXA-295, OXA-296, OXA-297, OXA-298, OXA-299, OXA-3, OXA-300, OXA-301, OXA-302, OXA-303, OXA-304, OXA-305, OXA-306, OXA-308, OXA-309, OXA-31, OXA-312, OXA-313, OXA-314, OXA-315, OXA-316, OXA-317, OXA-32, OXA-320, OXA-322, OXA-323, OXA-324, OXA-325, OXA-326, OXA-327, OXA-328, OXA-329, OXA-33, OXA-330, OXA-331, OXA-332, OXA-333, OXA-334, OXA-335, OXA-336, OXA-337, OXA-338, OXA-339, OXA-34, OXA-340, OXA-341, OXA-342, OXA-343, OXA-344, OXA-345, OXA-346, OXA-347, OXA-348, OXA-349, OXA-35, OXA-350, OXA-351, OXA-352, OXA-353, OXA-354, OXA-355, OXA-356, OXA-357, OXA-358, OXA-359, OXA-36, OXA-360, OXA-361, OXA-362, OXA-363, OXA-364, OXA-365, OXA-366, OXA-368, OXA-37, OXA-370, OXA-371, OXA-372, OXA-373, OXA-374, OXA-375, OXA-376, OXA-377, OXA-378, OXA-379, OXA-380, OXA-381, OXA-382, OXA-383, OXA-384, OXA-385, OXA-386, OXA-387, OXA-388, OXA-389, OXA-390, OXA-391, OXA-392, OXA-397, OXA-398, OXA-4, OXA-400, OXA-401, OXA-402, OXA-403, OXA-404, OXA-405, OXA-406, OXA-407, OXA-408, OXA-409, OXA-411, OXA-412, OXA-413, OXA-414, OXA-415, OXA-416, OXA-417, OXA-418, OXA-42, OXA-420, OXA-421, OXA-422, OXA-423, OXA-424, OXA-425, OXA-426, OXA-427, OXA-429, OXA-43, OXA-430, OXA-431, OXA-432, OXA-433, OXA-435, OXA-436, OXA-437, OXA-438, OXA-439, OXA-440, OXA-441, OXA-442, OXA-443, OXA-444, OXA-446, OXA-447, OXA-448, OXA-449, OXA-45, OXA-450, OXA-451, OXA-452, OXA-453, OXA-454, OXA-455, OXA-457, OXA-458, OXA-459, OXA-46, OXA-460, OXA-461, OXA-464, OXA-465, OXA-466, OXA-47, OXA-470, OXA-471, OXA-472, OXA-473, OXA-474, OXA-475, OXA-476, OXA-477, OXA-478, OXA-479, OXA-48, OXA-480, OXA-482, OXA-483, OXA-484, OXA-485, OXA-486, OXA-488, OXA-49, OXA-5, OXA-50, OXA-51, OXA-53, OXA-535, OXA-54, OXA-55, OXA-56, OXA-57, OXA-58, OXA-59, OXA-60, OXA-61, OXA-62, OXA-63, OXA-64, OXA-65, OXA-66, OXA-663, OXA-664, OXA-665, OXA-67, OXA-68, OXA-69, OXA-7, OXA-70, OXA-71, OXA-72, OXA-73, OXA-74, OXA-75, OXA-76, OXA-77, OXA-78, OXA-79, OXA-80, OXA-82, OXA-83, OXA-84, OXA-85, OXA-86, OXA-87, OXA-88, OXA-89, OXA-9, OXA-90, OXA-91, OXA-92, OXA-93, OXA-94, OXA-95, OXA-96, OXA-97, OXA-98, OXA-99, OXY-1-1, OXY-1-2, OXY-1-3, OXY-1-4, OXY-1-6, OXY-2-1, OXY-2-10, OXY-2-2, OXY-2-3, OXY-2-4, OXY-2-5, OXY-2-6, OXY-2-7, OXY-2-8, OXY-2-9, OXY-3-1, OXY-4-1, OXY-5-1, OXY-5-2, OXY-6-1, OXY-6-2, OXY-6-3, OXY-6-4, OpmB, OpmD, OpmH, OprA, OprJ, OprM, OprN, OprZ, PC1 beta-lactamase (bla7), PDC-1, PDC-10, PDC-2, PDC-3, PDC-4, PDC-5, PDC-6, PDC-7, PDC-73, PDC-74, PDC-75, PDC-76, PDC-77, PDC-78, PDC-79, PDC-8, PDC-80, PDC-81, PDC-82, PDC-83, PDC-84, PDC-85, PDC-86, PDC-87, PDC-88, PDC-89, PDC-9, PDC-90, PDC-91, PDC-92, PDC-93, PEDO-1, PEDO-2, PEDO-3, PER-1, PER-2, PER-3, PER-4, PER-5, PER-6, PER-7, PNGM-1, Pasteurella multocida 16S rRNA mutation conferring resistance to spectinomycin, Planobispora rosea EF-Tu mutants conferring resistance to inhibitor GE2270A, PmpM, PmrF, Propionibacteria 23S rRNA with mutation conferring resistance to macrolide antibiotics, Pseudomonas aeruginosa CpxR, Pseudomonas aeruginosa catB6, Pseudomonas aeruginosa catB7, Pseudomonas aeruginosa emrE, Pseudomonas aeruginosa gyrA and parC conferring resistance to fluoroquinolone, Pseudomonas aeruginosa gyrA conferring resistance to fluoroquinolones, Pseudomonas aeruginosa oprD with mutation conferring resistance to imipenem, Pseudomonas aeruginosa parE conferring resistance to fluoroquinolones, Pseudomonas aeruginosa soxR, Pseudomonas mutant PhoP conferring resistance to colistin, Pseudomonas mutant PhoQ conferring resistance to colistin, PvrR, QepA1, QepA2, QepA3, QepA4, QnrA1, QnrA2, QnrA3, QnrA4, QnrA5, QnrA6, QnrA7, QnrB1, QnrB10, QnrB11, QnrB12, QnrB13, QnrB14, QnrB15, QnrB16, QnrB17, QnrB18, QnrB19, QnrB2, QnrB20, QnrB21, QnrB22, QnrB23, QnrB24, QnrB25, QnrB26, QnrB27, QnrB28, QnrB29, QnrB3, QnrB30, QnrB31, QnrB32, QnrB33, QnrB34, QnrB35, QnrB36, QnrB37, QnrB38, QnrB4, QnrB40, QnrB41, QnrB42, QnrB43, QnrB44, QnrB45, QnrB46, QnrB47, QnrB48, QnrB49, QnrB5, QnrB50, QnrB54, QnrB55, QnrB56, QnrB57, QnrB58, QnrB59, QnrB6, QnrB60, QnrB61, QnrB62, QnrB64, QnrB65, QnrB66, QnrB67, QnrB68, QnrB69, QnrB7, QnrB70, QnrB71, QnrB72, QnrB73, QnrB74, QnrB8, QnrB9, QnrC, QnrD1, QnrD2, QnrS1, QnrS10, QnrS11, QnrS12, QnrS15, QnrS2, QnrS3, QnrS4, QnrS5, QnrS6, QnrS7, QnrS8, QnrS9, QnrVC1, QnrVC3, QnrVC4, QnrVC5, QnrVC6, QnrVC7, R39, RCP-1, ROB-1, RSA-1, RSA-2, RbpA, Rhodobacter sphaeroides ampC beta-lactamase, Rhodococcus fascians cmr, RlmA(II), Rm3, SAT-2, SAT-3, SAT-4, SFB-1, SFH-1, SHV-1, SHV-100, SHV-101, SHV-102, SHV-103, SHV-104, SHV-105, SHV-106, SHV-107, SHV-108, SHV-109, SHV-11, SHV-110, SHV-111, SHV-112, SHV-119, SHV-12, SHV-120, SHV-121, SHV-122, SHV-123, SHV-124, SHV-125, SHV-126, SHV-127, SHV-128, SHV-129, SHV-13, SHV-133, SHV-134, SHV-135, SHV-137, SHV-14, SHV-140, SHV-141, SHV-142, SHV-143, SHV-144, SHV-145, SHV-147, SHV-148, SHV-149, SHV-15, SHV-150, SHV-151, SHV-152, SHV-153, SHV-154, SHV-155, SHV-156, SHV-157, SHV-158, SHV-159, SHV-16, SHV-160, SHV-161, SHV-162, SHV-163, SHV-164, SHV-165, SHV-167, SHV-168, SHV-172, SHV-173, SHV-178, SHV-179, SHV-18, SHV-180, SHV-182, SHV-183, SHV-185, SHV-186, SHV-187, SHV-188, SHV-189, SHV-19, SHV-2, SHV-20, SHV-21, SHV-22, SHV-23, SHV-24, SHV-25, SHV-26, SHV-27, SHV-28, SHV-29, SHV-2A, SHV-3, SHV-30, SHV-31, SHV-32, SHV-33, SHV-34, SHV-35, SHV-36, SHV-37, SHV-38, SHV-39, SHV-40, SHV-41, SHV-42, SHV-43, SHV-44, SHV-45, SHV-46, SHV-48, SHV-49, SHV-5, SHV-50, SHV-51, SHV-52, SHV-53, SHV-55, SHV-56, SHV-57, SHV-59, SHV-6, SHV-60, SHV-61, SHV-62, SHV-63, SHV-64, SHV-65, SHV-66, SHV-67, SHV-69, SHV-7, SHV-70, SHV-71, SHV-72, SHV-73, SHV-74, SHV-75, SHV-76, SHV-77, SHV-78, SHV-79, SHV-8, SHV-80, SHV-81, SHV-82, SHV-83, SHV-84, SHV-85, SHV-86, SHV-89, SHV-9, SHV-92, SHV-93, SHV-94, SHV-95, SHV-96, SHV-97, SHV-98, SHV-99, SIM-1, SLB-1, SMB-1, SME-1, SME-2, SME-3, SME-4, SME-5, SPG-1, SPM-1, SRT-1, SRT-2, Salmonella enterica 16S rRNA (rrsD) mutation conferring resistance to spectinomycin, Salmonella enterica cmlA, Salmonella enterica gyrA conferring resistance to fluoroquinolones, Salmonella enterica gyrA with mutation conferring resistance to triclosan, Salmonella enterica parC conferring resistance to fluoroquinolones, Salmonella enterica ramR mutants, Salmonella enterica soxR with mutation conferring antibiotic resistance, Salmonella serovars gyrB conferring resistance to fluoroquinolone, Salmonella serovars parE conferring resistance to fluoroquinolones, Salmonella serovars soxS with mutation conferring antibiotic resistance, Sed-1, Serratia marcescens Omp1, Shigella flexneri chloramphenicol acetyltransferase, Shigella flexneri gyrA conferring resistance to fluoroquinolones, Shigella flexneri parC conferring resistance to fluoroquinolones, Staphylococcus aureus 23S rRNA with mutation conferring resistance to linezolid, Staphylococcus aureus FosB, Staphylococcus aureus GlpT with mutation conferring resistance to fosfomycin, Staphylococcus aureus UhpT with mutation conferring resistance to fosfomycin, Staphylococcus aureus agrA with mutation conferring resistance to daptomycin, Staphylococcus aureus cls conferring resistance to daptomycin, Staphylococcus aureus fusA with mutation conferring resistance to fusidic acid, Staphylococcus aureus fusE with mutation conferring resistance to fusidic acid, Staphylococcus aureus gyrA conferring resistance to fluoroquinolones, Staphylococcus aureus gyrB conferring resistance to aminocoumarin, Staphylococcus aureus ileS with mutation conferring resistance to mupirocin, Staphylococcus aureus menA with mutation conferring resistance to lysocin, Staphylococcus aureus mprF, Staphylococcus aureus mprF with mutation conferring resistance to daptomycin, Staphylococcus aureus murA with mutation conferring resistance to fosfomycin, Staphylococcus aureus norA, Staphylococcus aureus parC conferring resistance to fluoroquinolone, Staphylococcus aureus parE conferring resistance to aminocoumarin, Staphylococcus aureus parE conferring resistance to fluoroquinolones, Staphylococcus aureus pgsA mutations conferring resistance to daptomycin, Staphylococcus aureus rpoB mutants conferring resistance to daptomycin, Staphylococcus aureus rpoB mutants conferring resistance to rifampicin, Staphylococcus aureus rpoC conferring resistance to daptomycin, Staphylococcus aureus walK with mutation conferring resistance to daptomycin, Staphylococcus intermedius chloramphenicol acetyltransferase, Staphylococcus mupA conferring resistance to mupirocin, Staphylococcus mupB conferring resistance to mupirocin, Staphylococcys aureus LmrS, Streptococcus agalactiae mprF, Streptococcus mitis CdsA with mutation conferring daptomycin resistance, Streptococcus pneumoniae 23S rRNA mutation conferring resistance to macrolides and streptogramins antibiotics, Streptococcus pneumoniae 23S rRNA with mutation conferring resistance to macrolide antibiotics, Streptococcus pneumoniae PBP1a conferring resistance to amoxicillin, Streptococcus pneumoniae PBP2b conferring resistance to amoxicillin, Streptococcus pneumoniae PBP2x conferring resistance to amoxicillin, Streptococcus pneumoniae parC conferring resistance to fluoroquinolone, Streptococcus pyogenes folP with mutation conferring resistance to sulfonamides, Streptococcus suis chloramphenicol acetyltransferase, Streptomyces ambofaciens 23S rRNA with mutation conferring resistance to macrolide antibiotics, Streptomyces cinnamoneus EF-Tu mutants conferring resistance to elfamycin, Streptomyces lividans cmlR, Streptomyces rishiriensis parY mutant conferring resistance to aminocoumarin, TEM-1, TEM-10, TEM-101, TEM-102, TEM-104, TEM-105, TEM-106, TEM-107, TEM-108, TEM-109, TEM-11, TEM-110, TEM-111, TEM-112, TEM-113, TEM-114, TEM-115, TEM-116, TEM-117, TEM-118, TEM-12, TEM-120, TEM-121, TEM-122, TEM-123, TEM-124, TEM-125, TEM-126, TEM-127, TEM-128, TEM-129, TEM-130, TEM-131, TEM-132, TEM-133, TEM-134, TEM-135, TEM-136, TEM-137, TEM-138, TEM-139, TEM-141, TEM-142, TEM-143, TEM-144, TEM-145, TEM-146, TEM-147, TEM-148, TEM-149, TEM-15, TEM-150, TEM-151, TEM-152, TEM-153, TEM-154, TEM-155, TEM-156, TEM-157, TEM-158, TEM-159, TEM-16, TEM-160, TEM-162, TEM-163, TEM-164, TEM-166, TEM-167, TEM-168, TEM-169, TEM-17, TEM-171, TEM-176, TEM-177, TEM-178, TEM-182, TEM-183, TEM-184, TEM-185, TEM-186, TEM-187, TEM-188, TEM-189, TEM-19, TEM-190, TEM-191, TEM-192, TEM-193, TEM-194, TEM-195, TEM-196, TEM-197, TEM-198, TEM-199, TEM-2, TEM-20, TEM-201, TEM-205, TEM-206, TEM-207, TEM-208, TEM-209, TEM-21, TEM-211, TEM-213, TEM-214, TEM-215, TEM-216, TEM-217, TEM-219, TEM-22, TEM-220, TEM-24, TEM-26, TEM-28, TEM-29, TEM-3, TEM-30, TEM-33, TEM-34, TEM-4, TEM-40, TEM-42, TEM-43, TEM-45, TEM-47, TEM-48, TEM-49, TEM-52, TEM-53, TEM-54, TEM-55, TEM-57, TEM-59, TEM-6, TEM-60, TEM-63, TEM-67, TEM-68, TEM-7, TEM-70, TEM-71, TEM-72, TEM-73, TEM-75, TEM-76, TEM-78, TEM-79, TEM-8, TEM-80, TEM-81, TEM-82, TEM-83, TEM-84, TEM-85, TEM-86, TEM-87, TEM-88, TEM-89, TEM-90, TEM-91, TEM-92, TEM-93, TEM-94, TEM-95, TEM-96, THIN-B, TLA-1, TLA-2, TLA-3, TMB-1, TMB-2, TRU-1, TUS-1, TaeA, Tet(47), Tet(X3), Tet(X4), TolC, TriA, TriB, TriC, Type A NfxB, Type B NfxB, Ureaplasma urealyticum gyrB conferring resistance to fluoroquinolone, Ureaplasma urealyticum parC conferring resistance to fluoroquinolone, VCC-1, VEB-1, VEB-1b, VEB-2, VEB-3, VEB-4, VEB-5, VEB-6, VEB-7, VEB-8, VEB-9, VIM-1, VIM-10, VIM-11, VIM-12, VIM-13, VIM-14, VIM-15, VIM-16, VIM-17, VIM-18, VIM-19, VIM-2, VIM-20, VIM-23, VIM-24, VIM-25, VIM-26, VIM-27, VIM-28, VIM-29, VIM-3, VIM-30, VIM-31, VIM-32, VIM-33, VIM-34, VIM-35, VIM-36, VIM-37, VIM-38, VIM-39, VIM-4, VIM-42, VIM-43, VIM-5, VIM-6, VIM-7, VIM-8, VIM-9, VatI, Vibrio anguillarum chloramphenicol acetyltransferase, Vibrio cholerae OmpT, Vibrio cholerae OmpU, Vibrio cholerae varG, YojI, aacA43, aad(6), aadA, aadA10, aadA11, aadA12, aadA13, aadA14, aadA15, aadA16, aadA17, aadA2, aadA21, aadA22, aadA23, aadA24, aadA25, aadA27, aadA3, aadA4, aadA5, aadA6, aadA6/aadA10, aadA7, aadA8, aadA8b, aadA9, aadK, aadS, abcA, abeM, abeS, acrB, acrD, adeA, adeB, adeC, adeF, adeG, adeH, adeI, adeJ, adeK, adeL, adeN, adeR, adeS, almG, ampS, amrA, amrB, aphA15, apmA, arlR, arlS, armA, arnA, arr-1, arr-2, arr-3, arr-4, arr-5, arr-7, arr-8, bacA, baeR, baeS, basR, basS, bcr-1, bcrA, bcrB, bcrC, blaF, blaI, blaR1, blt, bmr, carA, carO, catA4, catA8, catB10, catB11, catB2, catB3, catB8, catB9, catI, catII, catII from Escherichia coli K-12, catIII, catP, catQ, catS, catV, cdeA, ceoA, ceoB, cepA, cfr(B), cfrA, cfrC, chrB, cipA, clbA, clbB, clbC, cicD, cmeA, cmeB, cmeC, cmeR, cmlA1, cmlA4, cmlA5, cmlA6, cmlA8, cmlB, cmlB1, cmlv, cmrA, cmx, cpaA, cphA2, cphA3, cphA4, cphA5, cphA6, cphA7, cphA8, cpxA, dfrA1, dfrA10, dfrA12, dfrA13, dfrA14, dfrA15, dfrA15b, dfrA16, dfrA17, dfrA18, dfrA19, dfrA20, dfrA21, dfrA22, dfrA23, dfrA24, dfrA25, dfrA26, dfrA27, dfrA28, dfrA29, dfrA3, dfrA30, dfrA32, dfrA3b, dfrA5, dfrA6, dfrA6 from Proteus mirabilis, dfrA7, dfrA8, dfrA9, dfrB1, dfrB2, dfrB3, dfrB4, dfrB5, dfrB6, dfrB7, dfrC, dfrD, dfrE, dfrF, dfrG, dfrI, dfrK, eatAv, efmA, efpA, efrA, efrB, emeA, emrA, emrB, emrD, emrK, emrR, emrY, emtA, eptA, erm(32), erm(40), erm(45), erm(46), ermZ, evgA, evgS, facT, farA, farB, fexA, floR, fusB, fusC, fusD, fusH, gadW, gadX, gimA, golS, hmrM, hp1181, hp1184, imiH, imiS, iri, kamB, kdpE, lfrA, lin, linG, lmrA, lmrB, lmrC, lmrD, lmrP, lnuA, lnuB, lnuC, lnuD, lnuE, lnuF, lnuG, lsaA, lsaB, lsaC, lsaE, macA, macB, marA, mdsA, mdsB, mdsC, mdtA, mdtB, mdtC, mdtE, mdtF, mdtG, mdtH, mdtM, mdtN, mdtO, mdtP, mecA, mecB, mecC, mecD, mecl, mecR1, mef(B), mefC, mefE, mel, mepA, mepR, mexM, mexN, mexP, mexQ, mexX, mexY, mfd, mfpA, mgrA, mgrB, mgtA, mphA, mphB, mphC, mphE, mphF, mphG, mphH, mphl, mphJ, mphK, mphL, mphM, mphN, mphO, msbA, msrA, msrB, msrC, msrE, mtrA, mtrC, mtrD, mtrE, mtrR, myrA, nalC, nalD, norA, norB, novA, npmA, oleB, oleC, oleD, olel, opcM, opmE, optrA, oqxA, oqxB, otr(A), otr(B), otrC, patA, patB, pexA, pgpB, plasmid-encoded cat (pp-cat), pmrA, porin OmpC, poxtA, pp-flo, qacA, qacB, qacH, qnrEl, qnrE2, ramA, rgt1438, rmtA, rmtB, rmtC, rmtD, rmtD2, rmtE, rmtE2, rmtF, rmtG, rmtH, rosA, rosB, rphA, rphB, rpoB2, rpsJ, salA, sav1866, sdiA, sgm, smeA, smeB, smeC, smeD, smeE, smeF, smeR, smeS, spd, srmB, sta, sul1, sul2, sul3, sul4, tap, tcmA, tcr3, tet(30), tet(31), tet(33), tet(35), tet(38), tet(39), tet(40), tet(41), tet(42), tet(43), tet(44), tet(45), tet(48), tet(49), tet(50), tet(51), tet(52), tet(53), tet(54), tet(55), tet(56), tet(59), tet(A), tet(B), tet(C), tet(D), tet(E), tet(G), tet(H), tet(J), tet(K), tet(L), tet(V), tet(W/N/W), tet(Y), tet(Z), tet32, tet34, tet36, tet37, tetA(46), tetA(58), tetA(60), tetA(P), tetB(46), tetB(58), tetB(60), tetB(P), tetM, tetO, tetQ, tetR, tetR(G), tetS, tetT, tetU, tetW, tetX, tlrB conferring tylosin resistance, tlrC, tmrB, tsnR, tva(A), ugd, vanA, vanB, vanC, vanD, vanE, vanF, vanG, vanHA, vanHB, vanHD, vanHF, vanHM, vanHO, vanl, vanJ, vanKl, vanL, vanM, vanN, vanO, vanRA, vanRB, vanRC, vanRD, vanRE, vanRF, vanRG, vanRl, vanRL, vanRM, vanRN, vanRO, vanSA, vanSB, vanSC, vanSD, vanSE, vanSF, vanSG, vanSl, vanSL, vanSM, vanSN, vanSO, vanTC, vanTE, vanTG, vanTN, vanTmL, vanTrL, vanUG, vanVB, vanWB, vanWG, vanWl, vanXA, vanXB, vanXD, vanXF, vanXl, vanXM, vanXO, vanXYC, vanXYE, vanXYG, vanXYL, vanXYN, vanYA, vanYB, vanYD, vanYF, vanYG1, vanYM, vanZA, vanZF, vatA, vatB, vatC, vatD, vatE, vatF, vatH, vga(E) Staphylococcus cohnii, vgaA, vgaALC, vgaB, vgaC, vgaD, vgaE, vgbA, vgbB, vgbC, vmlR, vph, y56 beta-lactamase, ykkC, ykkD, or any combination thereof.

In some embodiments, at least one component in the kit is provided in a desiccated or lyophilized form. In other embodiments, at least one component of the kit is provided in a solubilized form.

The kits provided herein are in suitable packaging. Suitable packaging includes, but is not limited to, vials, bottles, jars, flexible packaging, and the like. Also contemplated are packages for use in combination with a specific device. See “Devices for Sample Preparation and Sample Sequencing.” A kit may have a sterile access port (for example, the container may be an intravenous solution bag or a vial having a stopper pierceable by a hypodermic injection needle). The container may also have a sterile access port.

Kits optionally may provide additional components such as buffers and interpretive information. In some embodiments, the kit further comprises at least one buffer. Buffers suitable for the methods described herein have been described previously. In some embodiments, the kit can additionally comprise instructions for use in any of the methods described herein.

In some embodiment, the disclosure provides articles of manufacture comprising contents of the kits described above.

### V. Devices for Sample Preparation and Sample Sequencing

In some aspects, the disclosure relates to devices for sample preparation and/or sample sequencing. In some embodiments, the device comprises a sample preparation module. In some embodiments, the device comprises a sample sequencing module. In some embodiments, the device comprises a sample preparation module and a sample sequencing module.

### A. Device for Sample Preparation

Devices including apparatuses, cartridges (e.g., comprising channels (e.g., microfluidic channels)), and/or pumps (e.g., peristaltic pumps) for use in a process of preparing a sample for analysis are generally provided. Devices can be used in accordance with the instant disclosure to enable enrichment, concentration, manipulation, and/or detection of a target molecule from a biological sample. In some embodiments, devices and related methods are provided for automated processing of a sample to produce material for next generation sequencing and/or other downstream analytical techniques. Devices and related methods may be used for performing chemical and/or biological reactions, including reactions for nucleic acid and/or polypeptide processing in accordance with sample preparation or sample analysis processes described elsewhere herein.

In some embodiments, a sample preparation device is positioned to deliver or transfer to a sequencing module or device a target molecule or sample comprising a plurality of molecules (e.g., a target nucleic acid or a target polypeptide). In some embodiments, a sample preparation device is connected directly to (e.g., physically attached to) or indirectly to a sequencing device.

In some embodiments, a device comprise a sequence preparation module that is configured to receive one or more cartridges. In some embodiments, a cartridge comprises one or more reservoirs or reaction vessels configured to receive a fluid and/or contain one or more reagents used in a sample preparation process. In some embodiments, a cartridge comprises one or more channels (e.g., microfluidic channels) configured to contain and/or transport a fluid (e.g., a fluid comprising one or more reagents) used in a sample preparation process. Reagents include buffers, enzymatic reagents, polymer matrices, enrichment molecules, capture reagents, size-specific selection reagents, sequence-specific selection reagents, and/or purification reagents. Additional reagents for use in a sample preparation process are described elsewhere herein.

In some embodiments, a cartridge includes one or more stored reagents (e.g., of a liquid or lyophilized form suitable for reconstitution to a liquid form). The stored reagents of a cartridge include reagents suitable for carrying out a desired process and/or reagents suitable for processing a desired sample type. In some embodiments, a cartridge is a single-use cartridge (e.g., a disposable cartridge) or a multiple-use cartridge (e.g., a reusable cartridge). In some embodiments, a cartridge is configured to receive a user-supplied sample. The user-supplied sample may be added to the cartridge before or after the cartridge is received by the device, e.g., manually by the user or in an automated process.

In some embodiments, the device may facilitate enrichment of a target molecule in a process in accordance with the instant disclosure. See “Methods of Polypeptide Enrichment.” In this way, the device enables the leveraging of molecules to enrich for polypeptides of interest in a highly multiplexed fashion.

In some embodiments, a sample is enriched for a target molecule using an electropheretic method. In some embodiments, a sample is enriched for a target molecule using affinity SCODA. In some embodiments, a sample is enriched for a target molecule using field inversion gel electrophoresis (FIGE). In some embodiments, a sample is enriched for a target molecule using pulsed field gel electrophoresis (PFGE).

In some embodiments, a device comprises sample preparation module comprising a matrix used during enrichment (e.g., a porous media, electrophoretic polymer gel) comprising immobilized capture probes that bind (directly or indirectly) to target molecules present in the sample. In some embodiments, a matrix used during enrichment comprises 1, 2, 3, 4, 5, or more unique immobilized capture probes, each of which binds to a unique target molecule and/or bind to the same target molecule with different binding affinities.

In some embodiments, an immobilized capture probe is a polypeptide capture probe that binds to a target polypeptide or polypeptide fragment. For example, in some embodiments, an immobilized capture probe is an enrichment molecule as described herein.

In some embodiments, a polypeptide capture probe binds to a target polypeptide (or polypeptide fragment) with a binding affinity of 10⁻⁹ to 10⁻⁸ M, 10⁻⁸ to 10⁻⁷ M, 10⁻⁷ to 10⁻⁶ M, 10⁻⁶ to 10⁻⁵ M, 10⁻⁵ to 10⁻⁴ M, 10⁻⁴ to HP M, or 10⁻³ to 10⁻² M. In some embodiments, the binding affinity is in the picomolar to nanomolar range (e.g., between about 10⁻¹² and about 10⁻⁹ M). In some embodiments, the binding affinity is in the nanomolar to micromolar range (e.g., between about 10⁻⁹ and about 10⁻⁶ M). In some embodiments, the binding affinity is in the micromolar to millimolar range (e.g., between about 10⁻⁶ and about 10⁻³ M). In some embodiments, the binding affinity is in the picomolar to micromolar range (e.g., between about 10⁻¹² and about 10⁻⁶ M). In some embodiments, the binding affinity is in the nanomolar to millimolar range (e.g., between about 10⁻⁹ and about 10⁻³ M).

In some embodiments, an immobilized capture probe is an oligonucleotide capture probe that hybridizes to a target nucleic acid. In some embodiments, an oligonucleotide capture probe is at least 50%, 60%, 70%, 80%, 90% 95%, or 100% complementary to a target nucleic acid. In some embodiments, a single oligonucleotide capture probe may be used to enrich a plurality of related target nucleic acids (e.g., 2, 3, 4, 5, 6, 7, 8, 9, 10, 20, 30, 40, 50, or more related target nucleic acids) that share at least 50%, 60%, 70%, 80%, 90% 95%, or 99% sequence identity. Enrichment of a plurality of related target nucleic acids may allow for the generation of a metagenomic library. In some embodiments, an oligonucleotide capture probe may enable differential enrichment of related target nucleic acids. In some embodiments, an oligonucleotide capture probe may enable enrichment of a target nucleic acid relative to a nucleic acid of identical sequence that differs in its modification state (e.g., methylation state, acetylation state).

In some embodiments, for the purposes of enriching nucleic acid target molecules with a length of 0.5-2 kilobases, oligonucleotide capture probes may be covalently immobilized in an acrylamide matrix using a 5′ Acrydite moiety. In some embodiments, for the purposes of enriching larger nucleic acid target molecules (e.g., with a length of >2 kilobases), oligonucleotide capture probes may be immobilized in an agarose matrix. In some embodiments, oligonucleotide capture probes may be immobilized in an agarose matrix using thiol-epoxide chemistries (e.g., by covalently attached thiol-modified oligonucleotides to crosslinked agarose beads). Oligonucleotide capture probes linked to agarose beads can be combined and solidified within standard agarose matrices (e.g., at the same agarose percentage).

In some embodiments, multiple capture probes (e.g., populations of multiple capture probe types, e.g., that bind to deterministic target molecules of infectious agents such as adenovirus, staphylococcus, pneumonia, or tuberculosis) may be immobilized in an enrichment matrix. Application of a sample to an enrichment matrix with multiple deterministic capture probes may result in diagnosis of a disease or condition (e.g., presence of an infectious agent).

In some embodiments, a device may facilitate release of a target molecule from the enrichment matrix after removal of non-target molecules, in a process in accordance with the instant disclosure. In some embodiments, a target molecule may be released from the enrichment matrix by increasing the temperature of the enrichment matrix. Adjusting the temperature of the matrix further influences migration rate as increased temperatures provide a higher capture probe stringency, requiring greater binding affinities between the target molecule and the capture probe. In some embodiments, when enriching related target molecules, the matrix temperature may be gradually increased in a step-wise manner in order to release and isolate target molecules in steps of ever-increasing homology. This may allow for the sequencing of target polypeptides or target nucleic acids that are increasingly distant in their relation to an initial reference target molecule, enabling discovery of novel proteins (e.g., enzymes) or functions (e.g., enzymatic function or gene function). In some embodiments, when using multiple capture probes (e.g., multiple deterministic capture probes), the matrix temperature may be increased in a step-wise or gradient fashion, permitting temperature-dependent release of different target molecules and resulting in generation of a series of barcoded release bands that represent the presence or absence of control and target molecules.

Devices in accordance with the instant disclosure generally contain mechanical and electronic and/or optical components which can be used to operate a cartridge as described herein. In some embodiments, the device components operate to achieve and maintain specific temperatures on a cartridge or on specific regions of the cartridge. In some embodiments, the device components operate to apply specific voltages for specific time durations to electrodes of a cartridge. In some embodiments, the device components operate to move liquids to, from, or between reservoirs and/or reaction vessels of a cartridge. In some embodiments, the device components operate to move liquids through channel(s) of a cartridge, e.g., to, from, or between reservoirs and/or reaction vessels of a cartridge. In some embodiments, the device components move liquids via a peristaltic pumping mechanism (e.g., apparatus) that interacts with an elastomeric, reagent-specific reservoir or reaction vessel of a cartridge. In some embodiments, the device components move liquids via a peristaltic pumping mechanism (e.g., apparatus) that is configured to interact with an elastomeric component (e.g., surface layer comprising an elastomer) associated with a channel of a cartridge to pump fluid through the channel. Device components can include computer resources, for example, to drive a user interface where sample information can be entered, specific processes can be selected, and run results can be reported.

The following non-limiting example is meant to illustrate aspects of the devices, methods, and compositions described herein. The use of a sample preparation device in accordance with the instant disclosure may proceed with one or more of the following described steps. A user may open the lid of the device and insert a cartridge that supports the desired process. The user may then add a sample, which may be combined with a specific lysis solution, to a sample port on the cartridge. The user may then close the device lid, enter any sample specific information via a touch screen interface on the device, select any process specific parameters (e.g., range of desired size selection, desired degree of homology for target molecule capture, etc.), and initiate the sample preparation process run.

Following the run, the user may receive relevant run data (e.g., confirmation of successful completion of the run, run specific metrics, etc.), as well as process specific information (e.g., amount of sample generated, presence or absence of specific target sequence, etc.). Data generated by the run may be subjected to subsequent bioinformatics analysis, which can be either local or cloud based. Depending on the process, a finished sample may be extracted from the cartridge for subsequent use (e.g., genomic sequencing, qPCR quantification, cloning, etc.). The device may then be opened, and the cartridge may then be removed.

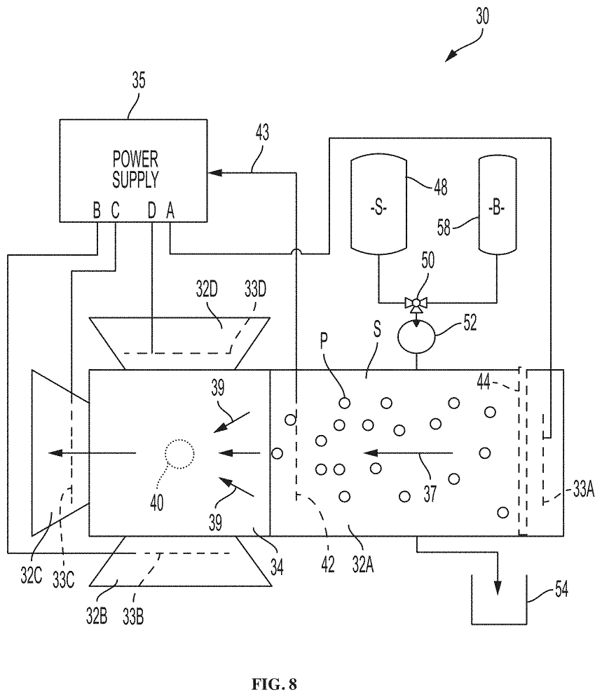

FIG. 8 provides an illustration depicting an exemplary apparatus for performing enrichment. See e.g., U.S. Pat. No. 8,608,929, the entirety of which is incorporated herein by reference.

### B. Device for Sequencing

Devices including apparatuses, cartridges (e.g., comprising channels (e.g., microfluidic channels)), and/or pumps (e.g., peristaltic pumps) for use in a process of sequencing a sample (e.g., an enriched sample) comprising polypeptides are also generally provided. Sequencing of nucleic acids or polypeptides in accordance with the instant disclosure, in some aspects, may be performed using a system that permits single molecule analysis and/or the sequencing of single molecules in parallel. The system may include a sequencing device and an instrument configured to interface with the sequencing device.

The sequencing device may include a sequencing module comprising an array of pixels, where individual pixels include a sample well and at least one photodetector. The sample wells of the sequencing device may be formed on or through a surface of the sequencing device and be configured to receive a sample placed on the surface of the sequencing device. In some embodiments, the sample wells are a component of a cartridge (e.g., a disposable or single-use cartridge) that can be inserted into the device. Collectively, the sample wells may be considered as an array of sample wells. The plurality of sample wells may have a suitable size and shape such that at least a portion of the sample wells receive a single target molecule or sample comprising a plurality of molecules (e.g., a target nucleic acid or a target polypeptide). In some embodiments, the number of molecules within a sample well may be distributed among the sample wells of the sequencing device such that some sample wells contain one molecule (e.g., a target nucleic acid or a target polypeptide) while others contain zero, two, or a plurality of molecules.

In some embodiments, a sequencing device is positioned to receive a sample comprising a plurality of molecules (e.g., one or more polypeptides of interest) from a sample preparation device. In some embodiments, a sequencing device is connected directly (e.g., physically attached to) or indirectly to a sample preparation device.

The sequencing device may include an array of pixels, where individual pixels include a sample well and at least one photodetector. The sample wells of the sequencing device may be formed on or through a surface of the sequencing device and be configured to receive a sample placed on the surface of the sequencing device. Collectively, the sample wells may be considered as an array of sample wells. The plurality of sample wells may have a suitable size and shape such that at least a portion of the sample wells receive a single sample (e.g., a single molecule, such as a polypeptide). In some embodiments, the number of samples within a sample well may be distributed among the sample wells of the sequencing device such that some sample wells contain one sample while others contain zero, two or more samples.

Excitation light is provided to the sequencing device from one or more light source, which may be external or internal to the sequencing device. Optical components of the sequencing device may receive the excitation light from the light source and direct the light towards the array of sample wells of the sequencing device and illuminate an illumination region within the sample well. In some embodiments, a sample well may have a configuration that allows for the sample to be retained in proximity to a surface of the sample well, which may ease delivery of excitation light to the sample and detection of emission light from the sample. A sample positioned within the illumination region may emit emission light in response to being illuminated by the excitation light. For example, the sample may be labeled with a fluorescent marker, which emits light in response to achieving an excited state through the illumination of excitation light. Emission light emitted by a sample may then be detected by one or more photodetectors within a pixel corresponding to the sample well with the sample being analyzed. When performed across the array of sample wells, which may range in number between approximately 10,000 pixels to 1,000,000 pixels according to some embodiments, multiple samples can be analyzed in parallel.

The sequencing device may include an optical system for receiving excitation light and directing the excitation light among the sample well array. The optical system may include one or more grating couplers configured to couple excitation light to the sequencing device and direct the excitation light to other optical components. The optical system may include optical components that direct the excitation light from a grating coupler towards the sample well array. Such optical components may include optical splitters, optical combiners, and waveguides. In some embodiments, one or more optical splitters may couple excitation light from a grating coupler and deliver excitation light to at least one of the waveguides. According to some embodiments, the optical splitter may have a configuration that allows for delivery of excitation light to be substantially uniform across all the waveguides such that each of the waveguides receives a substantially similar amount of excitation light. Such embodiments may improve performance of the sequencing device by improving the uniformity of excitation light received by sample wells of the sequencing device. Examples of suitable components, e.g., for coupling excitation light to a sample well and/or directing emission light to a photodetector, to include in a sequencing device are described in U.S. patent application Ser. No. 14/821,688, filed Aug. 7, 2015, titled “INTEGRATED DEVICE FOR PROBING, DETECTING AND ANALYZING MOLECULES,” and U.S. patent application Ser. No. 14/543,865, filed Nov. 17, 2014, titled “INTEGRATED DEVICE WITH EXTERNAL LIGHT SOURCE FOR PROBING, DETECTING, AND ANALYZING MOLECULES,” both of which are incorporated by reference in their entirety. Examples of suitable grating couplers and waveguides that may be implemented in the sequencing device are described in U.S. patent application Ser. No. 15/844,403, filed Dec. 15, 2017, titled “OPTICAL COUPLER AND WAVEGUIDE SYSTEM,” which is incorporated by reference in its entirety.

Additional photonic structures may be positioned between the sample wells and the photodetectors and configured to reduce or prevent excitation light from reaching the photodetectors, which may otherwise contribute to signal noise in detecting emission light. In some embodiments, metal layers which may act as a circuitry for the sequencing device, may also act as a spatial filter. Examples of suitable photonic structures may include spectral filters, a polarization filters, and spatial filters and are described in U.S. patent application Ser. No. 16/042,968, filed Jul. 23, 2018, titled “OPTICAL REJECTION PHOTONIC STRUCTURES,” which is incorporated by reference in its entirety.

Components located off of the sequencing device may be used to position and align an excitation source to the sequencing device. Such components may include optical components including lenses, mirrors, prisms, windows, apertures, attenuators, and/or optical fibers. Additional mechanical components may be included in the instrument to allow for control of one or more alignment components. Such mechanical components may include actuators, stepper motors, and/or knobs. Examples of suitable excitation sources and alignment mechanisms are described in U.S. patent application Ser. No. 15/161,088, filed May 20, 2016, titled “PULSED LASER AND SYSTEM,” which is incorporated by reference in its entirety. Another example of a beam-steering module is described in U.S. patent application Ser. No. 15/842,720, filed Dec. 14, 2017, titled “COMPACT BEAM SHAPING AND STEERING ASSEMBLY,” which is incorporated herein by reference. Additional examples of suitable excitation sources are described in U.S. patent application Ser. No. 14/821,688, filed Aug. 7, 2015, titled “INTEGRATED DEVICE FOR PROBING, DETECTING AND ANALYZING MOLECULES,” which is incorporated by reference in its entirety.

The photodetector(s) positioned with individual pixels of the sequencing device may be configured and positioned to detect emission light from the pixel's corresponding sample well. Examples of suitable photodetectors are described in U.S. patent application Ser. No. 14/821,656, filed Aug. 7, 2015, titled “INTEGRATED DEVICE FOR TEMPORAL BINNING OF RECEIVED PHOTONS,” which is incorporated by reference in its entirety. In some embodiments, a sample well and its respective photodetector(s) may be aligned along a common axis. In this manner, the photodetector(s) may overlap with the sample well within the pixel.

Characteristics of the detected emission light may provide an indication for identifying the marker associated with the emission light. Such characteristics may include any suitable type of characteristic, including an arrival time of photons detected by a photodetector, an amount of photons accumulated over time by a photodetector, and/or a distribution of photons across two or more photodetectors. In some embodiments, a photodetector may have a configuration that allows for the detection of one or more timing characteristics associated with a sample's emission light (e.g., luminescence lifetime). The photodetector may detect a distribution of photon arrival times after a pulse of excitation light propagates through the sequencing device, and the distribution of arrival times may provide an indication of a timing characteristic of the sample's emission light (e.g., a proxy for luminescence lifetime). In some embodiments, the one or more photodetectors provide an indication of the probability of emission light emitted by the marker (e.g., luminescence intensity). In some embodiments, a plurality of photodetectors may be sized and arranged to capture a spatial distribution of the emission light. Output signals from the one or more photodetectors may then be used to distinguish a marker from among a plurality of markers, where the plurality of markers may be used to identify a sample within the sample. In some embodiments, a sample may be excited by multiple excitation energies, and emission light and/or timing characteristics of the emission light emitted by the sample in response to the multiple excitation energies may distinguish a marker from a plurality of markers.

In operation, parallel analyses of samples within the sample wells are carried out by exciting some or all of the samples within the wells using excitation light and detecting signals from sample emission with the photodetectors. Emission light from a sample may be detected by a corresponding photodetector and converted to at least one electrical signal. The electrical signals may be transmitted along conducting lines in the circuitry of the sequencing device, which may be connected to an instrument interfaced with the sequencing device. The electrical signals may be subsequently processed and/or analyzed. Processing or analyzing of electrical signals may occur on a suitable computing device either located on or off the instrument.

The instrument may include a user interface for controlling operation of the instrument and/or the sequencing device. The user interface may be configured to allow a user to input information into the instrument, such as commands and/or settings used to control the functioning of the instrument. In some embodiments, the user interface may include buttons, switches, dials, and a microphone for voice commands. The user interface may allow a user to receive feedback on the performance of the instrument and/or sequencing device, such as proper alignment and/or information obtained by readout signals from the photodetectors on the sequencing device. In some embodiments, the user interface may provide feedback using a speaker to provide audible feedback. In some embodiments, the user interface may include indicator lights and/or a display screen for providing visual feedback to a user.

In some embodiments, the instrument may include a computer interface configured to connect with a computing device. The computer interface may be a USB interface, a FireWire interface, or any other suitable computer interface. A computing device may be any general purpose computer, such as a laptop or desktop computer. In some embodiments, a computing device may be a server (e.g., cloud-based server) accessible over a wireless network via a suitable computer interface. The computer interface may facilitate communication of information between the instrument and the computing device. Input information for controlling and/or configuring the instrument may be provided to the computing device and transmitted to the instrument via the computer interface. Output information generated by the instrument may be received by the computing device via the computer interface. Output information may include feedback about performance of the instrument, performance of the sequencing device, and/or data generated from the readout signals of the photodetector.

In some embodiments, the instrument may include a processing device configured to analyze data received from one or more photodetectors of the sequencing device and/or transmit control signals to the excitation source(s). In some embodiments, the processing device may comprise a general purpose processor, a specially-adapted processor (e.g., a central processing unit (CPU) such as one or more microprocessor or microcontroller cores, a field-programmable gate array (FPGA), an application-specific integrated circuit (ASIC), a custom integrated circuit, a digital signal processor (DSP), or a combination thereof). In some embodiments, the processing of data from one or more photodetectors may be performed by both a processing device of the instrument and an external computing device. In other embodiments, an external computing device may be omitted and processing of data from one or more photodetectors may be performed solely by a processing device of the sequencing device.

According to some embodiments, the instrument that is configured to analyze samples based on luminescence emission characteristics may detect differences in luminescence lifetimes and/or intensities between different luminescent molecules, and/or differences between lifetimes and/or intensities of the same luminescent molecules in different environments. The inventors have recognized and appreciated that differences in luminescence emission lifetimes can be used to discern between the presence or absence of different luminescent molecules and/or to discern between different environments or conditions to which a luminescent molecule is subjected. In some cases, discerning luminescent molecules based on lifetime (rather than emission wavelength, for example) can simplify aspects of the system. As an example, wavelength-discriminating optics (such as wavelength filters, dedicated detectors for each wavelength, dedicated pulsed optical sources at different wavelengths, and/or diffractive optics) may be reduced in number or eliminated when discerning luminescent molecules based on lifetime. In some cases, a single pulsed optical source operating at a single characteristic wavelength may be used to excite different luminescent molecules that emit within a same wavelength region of the optical spectrum but have measurably different lifetimes. An analytic system that uses a single pulsed optical source, rather than multiple sources operating at different wavelengths, to excite and discern different luminescent molecules emitting in a same wavelength region can be less complex to operate and maintain, more compact, and may be manufactured at lower cost.

Although analytic systems based on luminescence lifetime analysis may have certain benefits, the amount of information obtained by an analytic system and/or detection accuracy may be increased by allowing for additional detection techniques. For example, some embodiments of the systems may additionally be configured to discern one or more properties of a sample based on luminescence wavelength and/or luminescence intensity. In some implementations, luminescence intensity may be used additionally or alternatively to distinguish between different luminescent labels. For example, some luminescent labels may emit at significantly different intensities or have a significant difference in their probabilities of excitation (e.g., at least a difference of about 35%) even though their decay rates may be similar. By referencing binned signals to measured excitation light, it may be possible to distinguish different luminescent labels based on intensity levels.

According to some embodiments, different luminescence lifetimes may be distinguished with a photodetector that is configured to time-bin luminescence emission events following excitation of a luminescent label. The time binning may occur during a single charge-accumulation cycle for the photodetector. A charge-accumulation cycle is an interval between read-out events during which photo-generated carriers are accumulated in bins of the time-binning photodetector. Examples of a time-binning photodetector are described in U.S. patent application Ser. No. 14/821,656, filed Aug. 7, 2015, titled “INTEGRATED DEVICE FOR TEMPORAL BINNING OF RECEIVED PHOTONS,” which is incorporated herein by reference. In some embodiments, a time-binning photodetector may generate charge carriers in a photon absorption/carrier generation region and directly transfer charge carriers to a charge carrier storage bin in a charge carrier storage region. In such embodiments, the time-binning photodetector may not include a carrier travel/capture region. Such a time-binning photodetector may be referred to as a “direct binning pixel.” Examples of time-binning photodetectors, including direct binning pixels, are described in U.S. patent application Ser. No. 15/852,571, filed Dec. 22, 2017, titled “INTEGRATED PHOTODETECTOR WITH DIRECT BINNING PIXEL,” which is incorporated herein by reference.

In some embodiments, different numbers of fluorophores of the same type may be linked to different reagents in a sample, so that each reagent may be identified based on luminescence intensity. For example, two fluorophores may be linked to a first labeled affinity reagent and four or more fluorophores may be linked to a second labeled affinity reagent. Because of the different numbers of fluorophores, there may be different excitation and fluorophore emission probabilities associated with the different affinity reagents. For example, there may be more emission events for the second labeled affinity reagent during a signal accumulation interval, so that the apparent intensity of the bins is significantly higher than for the first labeled affinity reagent.

The inventors have recognized and appreciated that distinguishing nucleotides or any other biological or chemical samples based on fluorophore decay rates and/or fluorophore intensities may enable a simplification of the optical excitation and detection systems. For example, optical excitation may be performed with a single-wavelength source (e.g., a source producing one characteristic wavelength rather than multiple sources or a source operating at multiple different characteristic wavelengths). Additionally, wavelength discriminating optics and filters may not be needed in the detection system. Also, a single photodetector may be used for each sample well to detect emission from different fluorophores. The phrase “characteristic wavelength” or “wavelength” is used to refer to a central or predominant wavelength within a limited bandwidth of radiation (e.g., a central or peak wavelength within a 20 nm bandwidth output by a pulsed optical source). In some cases, “characteristic wavelength” or “wavelength” may be used to refer to a peak wavelength within a total bandwidth of radiation output by a source.

### EQUIVALENTS AND SCOPE

In the claims articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or all of the group members are present in, employed in, or otherwise relevant to a given product or process.

Furthermore, the invention encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group. It should it be understood that, in general, where the invention, or aspects of the invention, is/are referred to as comprising particular elements and/or features, certain embodiments of the invention or aspects of the invention consist, or consist essentially of, such elements and/or features. For purposes of simplicity, those embodiments have not been specifically set forth in haec verba herein.

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03. It should be appreciated that embodiments described in this document using an open-ended transitional phrase (e.g., “comprising”) are also contemplated, in alternative embodiments, as “consisting of” and “consisting essentially of” the feature described by the open-ended transitional phrase. For example, if the application describes “a composition comprising A and B,” the application also contemplates the alternative embodiments “a composition consisting of A and B” and “a composition consisting essentially of A and B.”

Where ranges are given, endpoints are included. Furthermore, unless otherwise indicated or otherwise evident from the context and understanding of one of ordinary skill in the art, values that are expressed as ranges can assume any specific value or sub-range within the stated ranges in different embodiments of the invention, to the tenth of the unit of the lower limit of the range, unless the context clearly dictates otherwise.

This application refers to various issued patents, published patent applications, journal articles, and other publications, all of which are incorporated herein by reference. If there is a conflict between any of the incorporated references and the instant specification, the specification shall control. In addition, any particular embodiment of the present invention that falls within the prior art may be explicitly excluded from any one or more of the claims. Because such embodiments are deemed to be known to one of ordinary skill in the art, they may be excluded even if the exclusion is not set forth explicitly herein. Any particular embodiment of the invention can be excluded from any claim, for any reason, whether or not related to the existence of prior art.

Those skilled in the art will recognize or be able to ascertain using no more than routine experimentation many equivalents to the specific embodiments described herein. The scope of the present embodiments described herein is not intended to be limited to the above Description, but rather is as set forth in the appended claims. Those of ordinary skill in the art will appreciate that various changes and modifications to this description may be made without departing from the spirit or scope of the present invention, as defined in the following claims.

The recitation of a listing of chemical groups in any definition of a variable herein includes definitions of that variable as any single group or combination of listed groups. The recitation of an embodiment for a variable herein includes that embodiment as any single embodiment or in combination with any other embodiments or portions thereof. The recitation of an embodiment herein includes that embodiment as any single embodiment or in combination with any other embodiments or portions thereof.

1. A method comprising:
(i) using a plurality of enrichment molecules to select a subset of polypeptides from a plurality of polypeptides, thereby generating an enriched sample comprising the subset of polypeptides; and
(ii) sequencing, in parallel, the polypeptides in the enriched sample.

2. A method comprising:
(i) contacting a plurality of polypeptides with a plurality of enrichment molecules to produce an enriched sample comprising a subset of the polypeptides in the plurality of polypeptides; and
(ii) sequencing, in parallel, the polypeptides of the enriched sample.

3. The method of claim 1, wherein (i) comprises:
(a) contacting a plurality of polypeptides with a plurality of enrichment molecules, wherein at least a subset of the enrichment molecules in the plurality of enrichment molecules binds to a subset of the polypeptides in the plurality of polypeptides, thereby generating a bound subset of polypeptides and an unbound subset of polypeptides; and
(b) isolating the bound subset of polypeptides to produce an enriched sample comprising a subset of the polypeptides in the plurality of polypeptides.

4. The method of claim 1, wherein (i) comprises:
(a) contacting a plurality of polypeptides with a plurality of enrichment molecules, wherein at least a subset of the enrichment molecules in the plurality of enrichment molecules binds to a subset of the polypeptides in the plurality of polypeptides, thereby generating a bound subset of polypeptides and an unbound subset of polypeptides; and
(b) isolating the unbound subset of polypeptides to produce an enriched sample comprising a subset of the polypeptides in the plurality of polypeptides.

5. The method of claim 1, wherein:
each of the enrichment molecules in the plurality of enrichment molecules comprise an antibody, an aptamer, or an enzyme; or
the enrichment molecules in a subset of the plurality of enrichment molecules comprise an antibody, an aptamer, or an enzyme.

6. (canceled)

7. The method of claim 1, wherein:
each of the enrichment molecules in the plurality of enrichment molecules is immobilized on a substrate; or
the enrichment molecules in a subset of the plurality of enrichment molecules are bound to a substrate,
optionally wherein the contacting of the plurality of polypeptides with the plurality of enrichment molecules occurs in (i) when a sample comprising the plurality of polypeptides contacts the substrate.

8.-9. (canceled)

10. The method of claim 7, wherein the substrate is selected from the group consisting of a surface, a bead, a particle, and a gel, optionally wherein:
the surface is a solid surface;
the bead is a magnetic bead; or
the particle is a magnetic particle.

11.-16. (canceled)

17. The method of claim 1, wherein (i) comprises:
(a) contacting a plurality of polypeptides with a first plurality of enrichment molecules, wherein at least a subset of the enrichment molecules in the first plurality of enrichment molecules binds to a subset of the polypeptides in the plurality of polypeptides, thereby generating a first bound subset of polypeptides and a first unbound subset of polypeptides;
(b) isolating the first bound subset of polypeptides or the first unbound subset of polypeptides of (a); and
(c) iteratively repeating steps (a) and (b) with one or more additional plurality of enrichment molecules to produce an enriched sample comprising a subset of the polypeptides in the plurality of polypeptides.

18.-19. (canceled)

20. The method of claim 1, wherein one or more of the polypeptides in the plurality of polypeptides is modified in vitro by contacting the polypeptides with a modifying agent prior, concurrently with, or subsequently to the contacting of the plurality of polypeptides with the plurality of enrichment molecules in (i), optionally wherein at least one polypeptide is modified by the addition of a post-translational modification.

21. The method of claim 20, wherein the modifying agent:
comprises a denaturant and at least one polypeptide is modified by denaturation;
blocks free carboxylate groups and at least one polypeptide is modified by blocking free carboxylate groups of the polypeptide;
blocks free thiol groups and at least one polypeptide is modified by blocking free thiol groups of the polypeptide;
comprises a cleaving agent and at least one polypeptide is modified by cleavage; or
a combination thereof.

22.-26. (canceled)

27. The method of claim 1, wherein (ii) comprises:
(a) contacting a single polypeptide molecule of the enriched sample with one or more terminal amino acid recognition molecules; and
(b) detecting a series of signal pulses indicative of association of the one or more terminal amino acid recognition molecules with successive amino acids exposed at a terminus of the single polypeptide while the single polypeptide is being degraded, thereby sequencing the single polypeptide molecule.

28. The method of claim 1, wherein (ii) comprises:
(a) contacting a single polypeptide molecule of the enriched sample with a composition comprising one or more terminal amino acid recognition molecules and a cleaving reagent; and
(b) detecting a series of signal pulses indicative of association of the one or more terminal amino acid recognition molecules with a terminus of the single polypeptide molecule in the presence of the cleaving reagent, wherein the series of signal pulses is indicative of a series of amino acids exposed at the terminus over time as a result of terminal amino acid cleavage by the cleaving reagent.

29. The method of claim 1, wherein (ii) comprises:
(a) identifying a first amino acid at a terminus of a single polypeptide molecule of the enriched sample;
(b) removing the first amino acid to expose a second amino acid at the terminus of the single polypeptide molecule, and
(c) identifying the second amino acid at the terminus of the single polypeptide molecule, wherein (a)-(c) are performed in a single reaction mixture.

30. The method of claim 1, wherein (ii) comprises:
(a) contacting a single polypeptide molecule of the enriched sample with one or more amino acid recognition molecules that bind to the single polypeptide molecule;
(b) detecting a series of signal pulses indicative of association of the one or more amino acid recognition molecules with the single polypeptide molecule under polypeptide degradation conditions; and
(c) identifying a first type of amino acid in the single polypeptide molecule based on a first characteristic pattern in the series of signal pulses.

31. The method of claim 1, wherein (ii) comprises:
(a) obtaining data during a polypeptide degradation process;
(b) analyzing the data to determine portions of the data corresponding to amino acids that are sequentially exposed at a terminus of the polypeptide during the degradation process; and
(c) outputting an amino acid sequence representative of the polypeptide.

32. The method of claim 1, wherein (ii) comprises:
(a) contacting a polypeptide of the enriched sample with one or more labeled affinity reagents that selectively bind one or more types of terminal amino acids at a terminus of the polypeptide; and
(b) identifying a terminal amino acid at the terminus of the polypeptide by detecting an interaction of the polypeptide with the one or more labeled affinity reagents.

33. The method of claim 1, wherein (ii) comprises:
(a) contacting a polypeptide in the enriched sample with one or more labeled affinity reagents that selectively bind one or more types of terminal amino acids at a terminus of the polypeptide;
(b) identifying a terminal amino acid at the terminus of the polypeptide by detecting an interaction of the polypeptide with the one or more labeled affinity reagents;
(c) removing the terminal amino acid; and
(d) repeating (a)-(c) one or more times at the terminus of the polypeptide to determine an amino acid sequence of the polypeptide,
optionally wherein the method further comprises:
after (a) and before (b), removing any of the one or more labeled affinity reagents that do not selectively bind the terminal amino acid; and/or
after (b) and before (c), removing any of the one or more labeled affinity reagents that selectively bind the terminal amino acid.

34.-38. (canceled)

39. A kit for performing the method of claim 1, wherein the kit comprises a plurality of enrichment molecules.

40.-45. (canceled)

46. A device comprising:
at least one hardware processor; and
at least one non-transitory computer-readable storage medium storing processor-executable instructions that, when executed by the at least one hardware processor, cause the at least one hardware processor to perform the method of claim 1.

47. (canceled)

48. A device comprising a sample preparation module configured to interface with one or more cartridge, each cartridge comprising: (a) one or more reservoirs or reaction vessels configured to receive a complex sample; (b) one or more sequence sample preparation reagents, wherein the sample preparation reagents comprise a plurality of enrichment molecules; and (c) a matrix comprising one or more immobilized capture probes.

49.-58. (canceled)
